(19) United States
(12) Patent Application Publication
(10) Pub. No.: US 2024/0412856 A1
(43) Pub. Date: Dec. 12, 2024

(54) SYSTEMS AND METHODS FOR EMS DISPATCH

(71) Applicant: ZOLL Medical Corporation, Chelmsford, MA (US)

(72) Inventors: Kurtis D. Mensch, Lafayette, CO (US); Jason C. Brock, Liberty, MO (US); Frederick W. Forester, Encitas, CA (US)

(21) Appl. No.: 18/735,454

(22) Filed: Jun. 6, 2024

Related U.S. Application Data
(60) Provisional application No. 63/471,873, filed on Jun. 8, 2023.

(51) Int. Cl. G16H 40/20; G01C 21/34; G01C 21/36; G06F 3/04817; H04W 4/90

(52) U.S. Cl. CPC G16H 40/20; G01C 21/3407; G01C 21/367; G06F 3/04817; H04W 4/90

(57) ABSTRACT

Systems and methods for cloud-based computer-aided dispatch (CAD) of emergency medical services (EMS) include receiving real-time EMS vehicle location tracking information from and sending an EMS vehicle assignment to a vehicle of an EMS agency, and communicatively coupling to a computing system associated with the EMS agency to provide services including request intake configured to receive a request for a patient's EMS trip, a dispatch GUI hosted at the EMS agency, and a dispatch code service for determining dispatch code(s) for the EMS trip based on the request. The dispatch GUI may include an interactive graphical map, and a data entry window for capturing trip information. Based on a user-selected position on the map, a portion of the trip information may automatically populate in the data entry window. The vehicle may be assigned using the trip information, the real-time vehicle location tracking information, the request, and the dispatch code(s).

### RELATED APPLICATIONS

This application claims priority to U.S. Provisional Patent Application Ser. No. 63/471,873 entitled “Systems and Methods for EMS Dispatch” filed Jun. 8, 2023. The above identified application is hereby incorporated by reference in its entirety.

### BACKGROUND

Traditional computer-aided dispatch (CAD) from emergency medical services (EMS) agencies is implemented as software executed by on-premises computers. These on-premises computers provide servers and connected workstations that enable human dispatchers to interact with CAD software in response to incoming calls for EMS agency services. These services may include emergency calls and non-emergency calls to pre-schedule patient transportation associated with medical services. The incoming calls may be telephonic. Such calls require a human dispatcher to speak with a caller to gather information and provide that information to the CAD software via entries to a user interface provided by the CAD software. In some cases, the incoming calls may be non-telephonic but rather in the form of a software-based request. For example, a hospital requiring discharge transport may access and schedule such transport via scheduling software that enables communications with an EMS agency.

In order to implement the dispatch of agency resources for either emergency or non-emergency calls, the CAD software prompts dispatchers to collect specific information that systematically determines the level of health services and/or emergency medical response that is required. The “call” refers to the incoming telephone calls, incoming requests via software, incoming fax requests as well as the response. The software then assesses the availability of agency resources appropriate for the required response or services and assigns, or dispatches, resources to the call or response. Thus, the CAD enables a dispatcher to process medical transfer and response requests in an accurate, consistent, and systematic manner.

### SUMMARY OF ILLUSTRATIVE EMBODIMENTS

In one aspect, the present disclosure relates to a cloud-based computer-aided dispatch (CAD) system for emergency medical services (EMS), the system including a navigation services engine configured to receive real-time EMS vehicle location tracking information from and send an EMS vehicle assignment to a computing device associated with at least one EMS agency vehicle associated with an EMS agency, and a dispatch engine configured to communicatively couple to a computing system associated with the EMS agency via a long-range communications network. The dispatch engine may include an EMS request intake engine configured to receive a request for EMS services for a patient, a dispatch graphical user interface (GUI) engine configured to host a dispatch GUI at the computing system associated with the EMS agency, where the dispatch GUI includes an interactive graphical map, and a data entry window including a number of data entry fields configured to capture EMS trip information for an EMS trip responsive to the request for EMS services, where the dispatch GUI engine is configured to automatically populate an item of EMS trip information in at least one data entry field based on a user selection of a position on the interactive graphical map, a dispatch code engine configured to determine at least one dispatch code for the EMS trip based on the request for EMS services, and a resource allocation engine. The resource allocation engine may be configured to assign the at least one EMS agency vehicle associated with the EMS agency to the EMS trip based at least on part on the automatically populated item of EMS trip information, the real-time EMS vehicle location tracking information, the request for the EMS services for the patient, and the at least one dispatch code, and provide the EMS vehicle assignment to the navigation services engine. The cloud-based CAD system may be configured for execution on a cloud-based distributed processing architecture.

In some embodiments, the dispatch engine is configured to communicate with the computing system associated with the EMS agency via an application programming interface (API). The dispatch GUI engine may be configured to identify a location corresponding to the position on the interactive graphical map, and display the identified location in the at least one data entry field configured to automatically populate the item of EMS trip information based on the position on the interactive graphical map. The identified location may include at least one of a street address or a name of a medical facility. The dispatch GUI engine may enable a user to edit the identified location. The at least one data entry field may be a patient transport location including a patient drop-off location for the EMS trip or a patient pick-up location for the EMS trip. The dispatch GUI engine may be configured to provide a transport location icon on the interactive graphical map at a map location corresponding to a geographic location of the patient transport location, and automatically populate the at least one data entry field with the patient transport location based on a user selection of the transport location icon. The dispatch GUI engine may be configured to accept a repositioning of the transport location icon at the interactive graphical map, and update the at least one data entry field to correspond with the repositioning. The user selection of the position on the interactive graphical map may cause the dispatch GUI engine to automatically capture the item of EMS trip information as global positioning satellite (GPS) coordinates for the position.

In some embodiments, the dispatch GUI engine is configured to provide a number of icons representing agency resource locations on the interactive graphical map, and display agency resource information at the dispatch GUI for a particular location in response to a user selection of a respective icon of the number of icons, wherein the respective icon is provided at the particular location. The resource allocation engine may be configured to access at least one post location for the EMS agency. The icons may include at least one post location icon. The dispatch GUI engine may be configured to receive the at least one post location for the EMS agency from the resource allocation engine, and provide the at least one post location icon on the interactive graphical map at the at least one post location. The at least one post location icon may indicate a quantity of vehicles currently located at the at least one post location. The dispatch GUI engine may be configured to automatically capture the item of EMS trip information based on a user selection of at least one post icon. The dispatch GUI engine may be configured to receive the real-time EMS vehicle location tracking information from the navigation services engine, provide a set of individual EMS vehicle icons each representing a respective EMS vehicle for the EMS agency, and automatically capture the item of EMS trip information based on a user selection of a respective EMS vehicle icon of the set of EMS vehicle icons. The icons may include at least one post location icon and at least one individual EMS vehicle location icon. The dispatch GUI engine may be configured to provide the at least one post location icon with a first shape and a first color, and provide the at least one individual EMS vehicle location icon with a second shape and a second color, where the second shape is different from the first shape or the second color is different from the first color or a combination thereof. The dispatch GUI engine may be configured to display one or more of crew information or vehicle information in response to a user selection of a particular icon of the number of icons. The user selection may include a mouse click or a cursor hover. The vehicle information may include a connectivity status representing one of availability of communications with a computing device associated with a transport vehicle or unavailability of communications with the computing device associated with the transport vehicle, where the transport vehicle corresponds to the particular icon. The dispatch GUI may be configured to provide filter options that enable a user to filter display of the number of icons to a particular type of EMS vehicle.

In some embodiments, the dispatch GUI engine is configured to display a log of the EMS trip, the log including a current status of the assigned EMS vehicle, and a time and time interval from assignment for a wheels rolling status. The dispatch GUI engine may be configured to receive the real-time EMS vehicle location tracking information from the navigation services engine, where the real-time EMS vehicle location tracking information includes a first location at a first time and at least a second location at a second time, evaluate a difference between the first location and the second location, and assign the time for the wheels rolling status based on the difference exceeding a predetermined threshold. The predetermined threshold may be within 2 to 10 meters. The log may include one or more of a time and time interval from assignment for an en route status, an at-scene status, a transporting status, or an at-destination status. The log of the EMS trip may include a time for a pre-scheduled patient transport. The log of the EMS trip may include one or more of a vehicle identification number, a vehicle service level, crew member identification, or contact information for a requester of the EMS services, or a connectivity status representing communications availability or unavailability between the computing device associated with the assigned EMS vehicle and the cloud-based CAD system. The connectivity status may register unavailability between the computing device associated with the assigned EMS vehicle and the cloud-based CAD system prior to a crew member of the assigned EMS vehicle logging into a navigation and dispatch application provided at the computing device associated with the assigned EMS vehicle. The log of the EMS trip may include a time stamp control, a cancel trip control, an unassign trip control, an add trip control, a return trip control, and an add leg control. The time stamp control, upon selection, may be configured to enable a user to enter a custom time stamp comprising a clock time and a text label, where the text label represents an action, status, or activity corresponding to the clock time. The dispatch GUI engine may be configured to display the log of the EMS trip in a scrollable window.

In some embodiments, the dispatch engine is configured to determine an estimated time en route (ETE) to a patient location for a number of EMS agency vehicles associated with the EMS agency. The dispatch GUI engine may be configured to provide an agency resource selection window including the ETE, and capture a user selection of a particular EMS agency vehicle based on the ETE. The resource allocation engine may be configured to assign the particular EMS agency vehicle to the EMS trip. The dispatch engine may be configured to receive a current location for at least one EMS agency vehicle from the navigation services engine, and determine the ETE based on the current location. The dispatch engine may be configured to request the ETE from the navigation services engine. The navigation services engine may be configured to determine the ETE based on a driving route between the current location and the patient location. The agency resource selection window may include a listing of agency resource vehicles ranked by ETE. The listing of agency resource vehicles may be ranked based further in part on at least one of a nature of a medical emergency experienced by the patient or at least one medical condition of the patient. Each respective agency resource vehicle of the listing of agency resource vehicles may include a vehicle identifier and an indication of vehicle capability, where the indication of vehicle capability includes a critical care transport (CCT) capability, a quick response vehicle (QRV) capability, and an advanced life support (ALS) capability. The listing of agency resource vehicles may include a control for reviewing further information corresponding to a given agency resource vehicle of the listing of agency resource vehicles, where the further information includes at least one of a listing of crew members, a status, one or more pending assignments, or a post location of the given agency resource vehicle.

In some embodiments, the dispatch engine includes the navigation services engine. The dispatch engine may be configured to communicate with the EMS agency via an application programming interface (API).

In some embodiments, the EMS agency is a first EMS agency at a first geographic location, the dispatch GUI is a first dispatch GUI, and the resource allocation engine includes a database of EMS vehicles associated at least with the first EMS agency and a second EMS agency at a second geographic location. The resource allocation engine may be configured to identify an EMS vehicle associated with the second EMS agency that is available for the EMS trip based on the at least one dispatch code, receive a patient location associated with the EMS trip, determine that the second geographic location is closer to the patient location than the first geographic location, transfer the EMS trip from the first EMS agency to the second EMS agency, and provide a transfer notification for the EMS trip at a second dispatch GUI at a computing system associated with the second EMS agency. The transfer notification may include an assignment of the EMS vehicle associated with the second EMS agency to the EMS trip. The transfer notification may include a request for the second EMS agency to allocate resources to the EMS trip without an assignment of the EMS vehicle. The database of EMS vehicles may include stored permission information that enables the resource allocation engine to transfer the EMS trip from the first EMS agency to the second EMS agency. The stored permission information may include data access guidelines that identify one or more types of EMS trip data that are exchangeable between the first EMS agency and the second EMS agency. A computing system associated with the second EMS agency and the computing system associated with the first EMS agency may be separate uncoupled computing systems such that a data exchange between the first EMS agency and the second EMS agency only occurs via the cloud-based distributed processing architecture. The resource allocation engine may be configured to convert EMS trip data from a first format or dispatch workflow associated with the first EMS agency to a second format or dispatch workflow associated with the second EMS agency.

In some embodiments, the EMS request intake engine is configured to receive the request for the EMS trip from one of a public safety answering point (PSAP), a web-based scheduling application for a medical facility, or a call originating at a patient location without PSAP routing, and associate the request with the EMS agency to enable the dispatch GUI engine to provide the request at the hosted dispatch GUI. The EMS request intake engine may be configured to receive the request for the EMS trip from the EMS agency and provide the request at the hosted dispatch GUI.

In some embodiments, the EMS request intake engine is configured to identify, using patient information, stored patient data corresponding to the patient information, where the stored patient data includes at least one of demographic data or medical data. The EMS request intake engine may be configured to provide, when assigning the at least one EMS agency vehicle, at least a portion of the stored patient data. The EMS request intake engine may be configured to return at least a portion of the stored patient data. The dispatch GUI engine may be configured to present, in the dispatch GUI, at least a portion of the stored patient data. The assigning of the at least one EMS agency vehicle may be based at least in part on at least one medical condition identified in the medical data.

In some embodiments, the dispatch code engine is configured to provide prompts including a series of questions, where the at least one dispatch code is determined based at least in part based on a series of responses to the series of questions. The dispatch GUI engine may be configured to present, in the dispatch GUI, a triage dialogue control, and the dispatch code engine may be initiated responsive to selection of the triage dialogue control. Initiating the dispatch code engine may include causing execution of the dispatch code engine on the computing system, where the dispatch code engine is installed on the computing system. The EMS request intake engine may be configured to automatically initiate the dispatch code engine upon creating a new trip corresponding to a medical emergency. The EMS request intake engine may be configured to store the series of questions. The EMS request intake engine may include the dispatch code engine.

In one aspect, the present disclosure relates to a cloud-based computer-aided dispatch (CAD) system for allocating response resources by automatically determining the nature of a medical emergency involving at least one person, the cloud-based CAD system including a navigation services engine configured to receive real-time EMS vehicle location tracking information from and send an EMS vehicle assignment to a computing device associated with at least one EMS agency vehicle associated with an EMS agency, a service connection engine configured to transfer at least a portion of a response to a request for EMS agency services to non-agency services including telehealth, a caller instruction engine configured to provide self-care instructions to a caller, and a caller interrogation engine. The caller interrogation engine may be configured to receive initial information for the request for EMS agency services, conduct an interrogation of the caller to determine a nature of the request, apply natural language processing (NLP) to answers from the caller to the interrogation, and based on results of the NLP, determine a resource allocation response to the request for EMS services. The cloud-based CAD system may include a resource allocation engine configured to receive the resource allocation response, determine resource allocation instructions based at least in part on the resource allocation response, select at least one of the navigation services engine, the service connection engine, or the caller instruction engine as a recipient of the resource allocation instructions based at least in part on the resource allocation response, and provide the resource allocation instructions to the selected recipient.

In some embodiments, the cloud-based CAD system includes a non-volatile storage medium storing at least one machine learning model configured to recognize background noise indicative of dangerous circumstances. The caller interrogation engine may be configured to perform machine learning analysis on a background audio portion of the answers from the caller, using the at least one machine learning model, to identify one or more background audio cues indicative of at least one of one or more situational factors, where the one or more situational factors include at least one dangerous circumstance. The at least one dangerous circumstance may include a threatening individual near a victim of the medical emergency.

In some embodiments, the cloud-based CAD system includes a non-volatile storage medium storing at least one machine learning model configured to recognize vocalizations indicative of dangerous circumstances. The caller interrogation engine may be configured to perform machine learning analysis on the answers from the caller, using the at least one machine learning model, to identify one or more vocal cues indicative of at least one of one or more situational factors, where the one or more situational factors include at least one dangerous circumstance. The one or more vocal cues may be indicative of at least one of fear, terror, or shock.

In some embodiments, applying the NLP includes identifying a sentiment of each respective answer of one or more of the answers from the caller, where the sentiment of the respective answer is indicative of i) at least one of one or more situational factors and/or ii) at least one of one or more medical symptom factors. The sentiment of the respective answer may be indicative of at least one of an emotion of the caller, a mental health factor of the caller, or a comprehension level of the caller to an instruction or a question posed to the caller.

In some embodiments, the caller is a victim of the medical emergency, and applying the NLP includes identifying a prosody of the answer, where the prosody of the answer is indicative of at least one of one or more medical symptom factors. The one or more medical symptom factors may include at least one of a stroke symptom, a shock symptom, or a mental health symptom.

In some embodiments, the caller interrogation engine is configured to access, from at least one third-party source, one or more current or recent events related to a vicinity nearby or including a location of the medical emergency, and analyze the one or more current or recent events to identify at least one of one or more situational factors, where the one or more situational factors include one or more potentially dangerous situations. The caller interrogation engine may be configured to automatically derive a location of the medical emergency from positioning data of a mobile device of the caller.

In some embodiments, the cloud-based CAD system includes a non-volatile storage medium storing interrogation records, each interrogation record including a respective limited answer question of a number of limited answer questions, and, for at least one answer of a set of potential answers to each question of the number of limited answer questions, a corresponding medical condition indicator, dispatch codes, and a set of interrogation rules identifying, for each medical condition indicator or set of medical condition indicators, a corresponding dispatch code of the number of dispatch codes for dispatching appropriate treatment to an individual exhibiting the respective medical condition indicator or set of medical condition indicators. The cloud-based CAD system may include at least one machine learning model configured to select a best next question of the number of limited answer questions based on a question and response history including one or more limited answer questions of the number of limited answer questions. Conducting the interrogation may include, for each iteration of a number of iterations, presenting, for verbal response, a respective question of the number of limited answer questions to the caller. Applying the NLP may include analyzing one or more words of each answer of the answers from the caller to identify a matching answer of the set of potential answers to the respective question, determining whether the matching answer corresponds to a given medical condition indicator, storing, in an interrogation history, an identification of the respective question, the matching answer, and any corresponding medical condition indicator, where the resource allocation response is determined based at least in part on the corresponding medical condition indicator, and using the at least one machine learning model, performing machine learning analysis on the interrogation history to determine a next question of the number of limited answer questions to present to the caller. Applying the NLP may include, when the matching answer corresponds to the given medical condition indicator, applying the set of interrogation rules to the given medical condition indicator and any other medical condition indicator stored in the interrogation history to identify a corresponding dispatch code, and if a corresponding one or more dispatch codes is identified, ending conducting the interrogation, and providing, to the selected recipient, the corresponding one or more dispatch codes. The limited answer questions may follow one or more protocols of the International Academies of Emergency Dispatch (IAED). The initial information may include location information. Presenting the respective question may include automatically presenting a recording of the respective question via an audible communication connection with the caller. Presenting the respective question may include presenting, at a user interface screen for reading by an operator to the caller, the respective question. Conducting the interrogation may include, for each iteration, obtaining, responsive to the respective question, a file including a recording of an answer to the respective question, where applying the NLP to the answer includes applying the NLP on the file. Conducting the interrogation may include determining a language of the caller. Performing the machine learning analysis may include performing the machine learning analysis further based on demographic information of a patient, where the initial information includes the demographic information. The set of potential answers for at least a portion of the number of limited answer questions may consist of yes, no, and unsure. Each dispatch code of the number of dispatch codes may correspond to a medical condition descriptor and a priority indicator. The cloud-based CAD system may include a machine learning model training engine configured to train the at least one machine learning model using previously stored interrogation histories including a number of questions and answers.

In some embodiments, the recipient of the resource allocation instructions is the navigation services engine, and the resource allocation instructions include an assignment of at least one EMS agency vehicle to the request for EMS agency services. The recipient of the resource allocation instructions may be the caller instruction engine. The resource allocation instructions may include one or more of an identification of at least one medical condition, a period of time for monitoring for symptoms in the at least one person, one or more symptoms to monitor for in the at least one person, one or more interventions for the at least one person, a recommendation for obtaining non-emergency assistance, or a phone number for referral to a non-emergency medical service.

In some embodiments, the recipient of the resource allocation instructions is the service connection engine, and the resource allocation instructions include one or more of indication of a type of service, call forwarding information to connect to a medical service, identification of a telehealth service, identification of a telehealth professional, or identification of a community intervention resource. The resource allocation instructions may be configured to coordinate a communications connection between the caller and/or the at least one person and the telehealth service or the telehealth professional. Coordinating the communications connection may include establishing a call-back used for the telehealth professional to contact the individual. The non-agency services may include one or more of mental health services, social services, community-based intervention services, or community-based outreach services.

In one aspect, the present disclosure relates to a cloud-based computer-aided dispatch (CAD) system for coordinating medical professional support responsive to a medical event involving a patient, the cloud-based CAD system including a navigation services engine configured to receive real-time EMS vehicle location tracking information from and send an EMS vehicle assignment to a computing device associated with at least one EMS agency vehicle associated with an EMS agency, and a dispatch engine configured to communicate with the EMS agency via an application programming interface (API). The dispatch engine may include an EMS request intake engine configured to receive a request for an EMS trip for a patient, a network communications interface configured to communicatively couple the cloud-based CAD system to a computing system associated with the EMS agency via a long-range communications network, a dispatch graphical user interface (GUI) engine configured to host a dispatch GUI at the computing system associated with the EMS agency, a dispatch code engine configured to determine at least one dispatch code for the EMS trip based on the request for the EMS trip, a service connection engine configured to communicatively couple to a telehealth service scheduler, and obtain telehealth support information from the telehealth service scheduler, and a resource allocation engine. The resource allocation engine may be configured to assign the at least one EMS agency vehicle associated with the EMS agency to the EMS trip based at least on part on the real-time EMS vehicle location tracking information, the request for the EMS trip for the patient, and the at least one dispatch code, and provide the EMS vehicle assignment and the telehealth support information to the navigation services engine. The cloud-based CAD system may be configured for execution on a cloud-based distributed processing architecture.

In some embodiments, the cloud-based CAD system includes a non-volatile data store including medical professional records corresponding to a number of medical professionals, each medical professional record identifying a name, contact information, availability, and one or more qualifications, and a set of rules for matching a number of medical condition descriptors with a number of medical professional qualifications. The service connection engine may be configured to obtain medical information for the patient, the medical information including one or more medical condition descriptors of the number of medical condition descriptors, determine, from the medical information, a transport requirement of the medical event, where the transport requirement includes transportation types comprising no transport required, transport required to the patient for on-site assistance, and transport required for moving the patient from a current location to a medical facility, identify, based at least in part on the one or more medical condition descriptors, at least one medical professional of the number of medical professionals for providing remote healthcare support to the patient, select, from the at least one medical professional, a selected medical professional for providing the remote healthcare support, where selecting includes determining a time for the remote healthcare support, and coordinate a communications connection for providing the remote healthcare support.

In some embodiments, the remote healthcare support includes on-site emergency medical team assistance with medical professional support, and coordinating the communications connection includes establishing a communications link between the at least one EMS agency vehicle and the selected medical professional. Selecting the selected medical professional may include matching a qualification of the selected medical professional with an accreditation or employee type of at least one member of an emergency medical team corresponding to the at least one EMS agency vehicle, such that the at least one member of the emergency medical team is qualified to provide a medical service when monitored by the selected medical professional. Establishing the communications link may include establishing a video communications link. The communications link may be established between an emergency medical vehicle communications system and the selected medical professional. The remote healthcare support may include en route emergency transport crew assistance with medical professional support, and coordinating the communications connection may include establishing a communications link between an emergency medical vehicle communications system and the selected medical professional. Determining the time for the remote healthcare support may include accessing a pre-scheduled patient transport request. The one or more qualifications may include a medical specialty, an employee type, or an accreditation. A portion of the number of medical professionals may be accredited to provide mental health services.

In some embodiments, the non-volatile data store further includes transportation data regarding a number of EMS agency vehicles, and the dispatch engine is configured to select, from the number of EMS agency vehicles, a selected EMS agency vehicle configured to provide the remote healthcare support. The selected EMS agency vehicle may be configured to support a communications link between the selected medical professional and a communications system of the selected EMS agency vehicle. The communications system may be configured to support audio and video communications. The communications system may be configured to support electronic medical record data transfer to the selected medical professional. The communications system may be configured to support real-time data transfer of monitoring data from one or more pieces of medical equipment configured to monitor patient functions.

In some embodiments, the service connection engine is configured to determine, from the medical information, a priority of the medical event, and select the selected professional based further in part on the priority of the medical event. Coordinating the communications connection may include establishing, at the time, at least one communications link between the selected medical professional and an EMS agency vehicle deployed to provide the remote healthcare support to the patient. The at least one communications link may include an audio-visual link. The at least one communications link may include a real-time data link for sharing monitoring data collected by sensors of medical equipment used on the patient.

In one aspect, the present disclosure relates to a cloud-based computer-aided dispatch (CAD) system including at least one non-transitory, processor-readable storage medium having stored thereon processor-readable instructions for emergency medical services (EMS) dispatch services, the processor-readable instructions being configured to cause at least one processor to receive real-time EMS vehicle location tracking information from and send an EMS vehicle assignment to a computing device associated with at least one EMS agency vehicle associated with an EMS agency, communicatively couple to a computing system associated with the EMS agency via a long-range communications network, receive a request for EMS services for a patient, host a dispatch GUI at the computing system associated with the EMS agency, where the dispatch GUI includes an interactive graphical map and a data entry window including a number of data entry fields configured to capture EMS trip information, automatically populate an item of EMS trip information in at least one data entry field based on a user selection of a position on the interactive graphical map, determine at least one dispatch code for the EMS services based on the request for the EMS services, and assign the at least one EMS agency vehicle associated with the EMS agency to the EMS services based at least on part on the automatically populated item of EMS trip information, the real-time EMS vehicle location tracking information, the request for the EMS services for the patient, and the at least one dispatch code, and generate the EMS vehicle assignment for an EMS trip corresponding to the request for EMS services. The cloud-based CAD system may be configured for execution on a cloud-based distributed processing architecture.

In some embodiments, the at least one processor is configured to communicate with the computing system associated with the EMS agency via an application programming interface (API). The at least one processor may be configured to identify a location corresponding to the position on the interactive graphical map, and display the identified location in the at least one data entry field configured to automatically populate the item of EMS trip information based on the user selected position on the interactive graphical map. The identified location may include at least one of a street address or a name of a medical facility. The at least one processor may be configured to enable a user to edit the identified location. The at least one data entry field may be a patient transport location including a patient drop-off location for the EMS services or a patient pick-up location for the EMS services. The at least one processor may be configured to provide a transport location icon on the interactive graphical map at a map location corresponding to a geographic location of the patient transport location, and automatically populate the at least one data entry field with the patient transport location based on a user selection of the transport location icon. The at least one processor may be configured to accept a repositioning of the transport location icon at the interactive graphical map, and update the at least one data entry field to correspond with the repositioning. The user selection of the position on the interactive graphical map may cause the at least one processor to automatically capture the item of EMS trip information as global positioning satellite (GPS) coordinates for the position.

In some embodiments, the at least one processor is configured to provide a number of icons representing agency resource locations on the interactive graphical map, and display agency resource information at the dispatch GUI for a particular location in response to a user selection of a respective icon of the number of icons, wherein the respective icon is provided at the particular location. The at least one processor may be configured to access at least one post location for the EMS agency, where the number of icons includes at least one post location icon, receive the at least one post location for the EMS agency, and provide the at least one post location icon on the interactive graphical map at the at least one post location. The at least one post location icon may indicate a quantity of vehicles currently located at the at least one post location. The at least one processor may be configured to automatically capture the item of EMS trip information based on a user selection of at least one post icon. The at least one processor may be configured to receive the real-time EMS vehicle location tracking information, provide a set of individual EMS vehicle icons each representing a respective EMS vehicle for the EMS agency, and automatically capture the item of EMS trip information based on a user selection of at least one post icon. The icons may include at least one post location icon and at least one individual EMS vehicle location icon. The at least one processor may be configured to provide the at least one post location icon with a first shape and a first color, and provide the at least one individual EMS vehicle icon with a second shape and a second color, where the second shape is different from the first shape or the second color is different from the first color or a combination thereof. The at least one processor may be configured to display one or more of crew information or vehicle information in response to a user selection of a particular icon of the number of icons. The user selection may include a mouse click or a cursor hover. The vehicle information may include a connectivity status representing one of availability of communications with a computing device associated with a transport vehicle or unavailability of communications with the computing device associated with the transport vehicle, where the transport vehicle corresponds to the particular icon. The dispatch GUI may be configured to provide filter options that enable a user to filter the display of the number of icons to a particular type of EMS vehicle.

In some embodiments, the at least one processor is configured to display a log of the EMS trip, the log including a current status of the assigned EMS vehicle, and a time and time interval from assignment for a wheels rolling status. The at least one processor may be configured to receive the real-time EMS vehicle location tracking information, where the real-time EMS vehicle location tracking information includes a first location at a first time and at least a second location at a second time, evaluate a difference between the first location and the second location, and assign the time for the wheels rolling status based on the difference exceeding a predetermined threshold. The predetermined threshold may be within 2 to 10 meters. The log may include one or more of a time and time interval from assignment for an en route status, an at-scene status, a transporting status, or an at-destination status. The log of the EMS trip may include a time for a pre-scheduled patient transport. The log of the EMS trip may include one or more of a vehicle identification number, a vehicle service level, crew member identification, contact information for a requester of the EMS services, or connectivity status representing communications availability or unavailability between the computing device associated with the assigned EMS vehicle and the cloud-based CAD system. The connectivity status may register unavailability between the computing device associated with the assigned EMS vehicle and the cloud-based CAD system prior to a crew member of the assigned EMS vehicle logging into a navigation and dispatch application provided at the computing device associated with the assigned EMS vehicle. The log of the EMS trip may include a time stamp control, a cancel trip control, an unassign trip control, an add trip control, a return trip control, and an add leg control. The time stamp control, upon selection, may be configured to enable a user to enter a custom time stamp including a clock time and a text label, where the text label represents an action, status, or activity corresponding to the clock time. The at least one processor may be configured to display the log of the EMS trip in a scrollable window.

In some embodiments, the at least one processor is configured to determine an estimated time en route (ETE) to a patient location for a number of EMS agency vehicles associated with the EMS agency, provide an agency resource selection window including the ETE, capture a user selection of a particular EMS agency vehicle based on the ETE, and assign the particular EMS agency vehicle to the EMS trip. The at least one processor may be configured to receive a current location for at least one EMS agency vehicle, and determine the ETE based on the current location. The at least one processor may be configured to determine the ETE based on a driving route between the current location and the patient location. The agency resource selection window may include a listing of agency resource vehicles ranked by ETE. The listing of agency resource vehicles may be ranked based further in part on at least one of a nature of a medical emergency experienced by the patient or at least one medical condition of the patient. Each respective agency resource vehicle of the listing of agency resource vehicles may include a vehicle identifier and an indication of vehicle capability, where the indication of vehicle capability includes a critical care transport (CCT) capability, a quick response vehicle (QRV) capability, and an advanced life support (ALS) capability. The listing of agency resource vehicles may include a control for reviewing further information corresponding to a given agency resource vehicle of the listing of agency resource vehicles, where the further information includes at least one of a listing of crew members, a status, one or more pending assignments, or a post location of the given agency resource vehicle.

In some embodiments, the at least one processor is configured to communicate with the EMS agency via an application programming interface (API). The cloud-based CAD system may include a database of EMS vehicles associated at least with the EMS agency and a second EMS agency at a second geographic location, where the EMS agency is a first EMS agency at a first geographic location, the dispatch GUI is a first dispatch GUI, and the at least one processor is configured to identify an EMS vehicle associated with the second EMS agency that is available for the EMS trip based on the at least one dispatch code, receive a patient location associated with the EMS trip, determine that the second geographic location is closer to the patient location than the first geographic location, transfer the EMS trip from the first EMS agency to the second EMS agency, and provide a transfer notification for the EMS trip at a second dispatch GUI at a computing system associated with the second EMS agency. The transfer notification may include an assignment of the EMS vehicle associated with the second EMS agency to the EMS trip. The transfer notification may include a request for the second EMS agency to allocate resources to the EMS trip without an assignment of the EMS vehicle. The database of EMS vehicles may include stored permission information that enables the at least one processor to transfer the EMS trip from the first EMS agency to the second EMS agency. The stored permission information may include data access guidelines that identify one or more types of EMS trip data that are exchangeable between the first EMS agency and the second EMS agency. A computing system associated with the second EMS agency and the computing system associated with the first EMS agency may be separate uncoupled computing systems such that a data exchange between the first EMS agency and the second EMS agency only occurs via the cloud-based distributed processing architecture. The at least one processor may be configured to convert EMS trip data from a first format or dispatch workflow associated with the first EMS agency to a second format or dispatch workflow associated with the second EMS agency.

In some embodiments, the at least one processor is configured to receive the request for the EMS trip from one of a public safety answering point (PSAP), a web-based scheduling application for a medical facility, or a call originating at a patient location without PSAP routing, associate the request with the EMS agency, and provide the request at the hosted dispatch GUI. The at least one processor may be configured to receive the request for the EMS trip from the EMS agency and provide the request at the hosted dispatch GUI.

In some embodiments, the at least one processor is configured to identify, using patient information, stored patient data corresponding to the patient information, where the stored patient data includes at least one of demographic data or medical data. The at least one processor may be configured to provide, when assigning the at least one EMS agency vehicle, at least a portion of the stored patient data. The at least one processor may be configured to present, in the dispatch GUI, at least a portion of the stored patient data. The assigning of the at least one EMS agency vehicle may be based at least in part on at least one medical condition identified in the medical data.

In some embodiments, at least one processor is configured to provide prompts including a series of questions, where the at least one dispatch code is determined based at least in part based on a series of responses to the series of questions. The at least one processor may be configured to present, in the dispatch GUI, a triage dialogue control, and determine a dispatch code responsive to selection of the triage dialogue control. Determining the at least one dispatch code may include causing execution of dispatch code instructions installed on the computing system. The at least one processor may be configured to automatically determine the at least one dispatch code upon creating a new trip corresponding to a medical emergency. The at least one processor may be configured to store the series of questions.

In one aspect, the present disclosure relates to a cloud-based computer-aided dispatch (CAD) system including at least one non-transitory, processor-readable storage medium having stored thereon processor-readable instructions for emergency medical services (EMS) dispatch services, the processor-readable instructions being configured to cause at least one processor to communicatively couple with a mobile computing device associated with an EMS agency vehicle, receive real-time EMS vehicle location tracking information from the mobile computing device associated with the EMS agency vehicle, receive a request for EMS services for a patient, generate a pending EMS trip assignment in response to the request for EMS services, communicatively couple to a computing system associated with an EMS agency via a long-range communications network, host a dispatch graphical user interface (GUI) at the computing system associated with the EMS agency, where the dispatch GUI includes an interactive graphical map and a data entry window including a number of data entry fields, automatically capture at least one item of EMS trip information in at least one data entry field of the number of data entry fields based on a user selection of a position or icon from the interactive graphical map, populate the pending EMS trip assignment based at least on part on the automatically captured at least one item of EMS trip information, the real-time EMS vehicle location tracking information, and the request for the EMS services for the patient, where the pending EMS trip assignment includes an identification of at least one EMS agency vehicle, send the pending EMS trip assignment to the mobile computing device associated with the EMS agency vehicle, receive a trip acceptance from the mobile computing device associated with the EMS agency vehicle, and generate an EMS agency vehicle assignment of the EMS agency vehicle to the request for EMS services in response to the trip acceptance. The cloud-based CAD system may be configured for execution on a cloud-based distributed processing architecture.

In some embodiments, the processor-readable instructions are configured to cause the at least one processor to identify a location corresponding to the position on the interactive graphical map, and display the identified location in the at least one data entry field configured to automatically capture the item of EMS trip information based on the user selected position on the interactive graphical map. The at least one data entry field may be a patient transport location including a patient drop-off location for the EMS trip or a patient pick-up location for the EMS trip, and the processor-readable instructions may be configured to cause the at least one processor to provide a transport location icon on the interactive graphical map at a map location corresponding to a geographic location of the patient transport location, and automatically populate the at least one data entry field with the patient transport location based on a user selection of the transport location icon. The processor-readable instructions may be configured to cause the at least one processor to accept a repositioning of the transport location icon at the interactive graphical map, and update the at least one data entry field to correspond with the repositioning.

In some embodiments, the user selection of the position on the interactive graphical map causes the at least one processor to automatically capture the item of EMS trip information as global positioning satellite (GPS) coordinates for the position. The processor-readable instructions may be configured to cause the at least one processor to provide a number of icons representing agency resource locations on the interactive graphical map, and display agency resource information at the dispatch GUI for a particular location in response to a user selection of a respective icon of the number of icons, where the respective icon is provided at the particular location. The processor-readable instructions may be configured to cause the at least one processor to access at least one post location for the EMS agency, and provide at least one post location icon on the interactive graphical map at the at least one post location. The at least one post location icon may indicate a quantity of vehicles currently located at the at least one post location. The processor-readable instructions may be configured to cause the at least one processor to automatically capture the item of EMS trip information based on a user selection of at least one post location icon. The processor-readable instructions may be configured to cause the at least one processor to provide a set of individual EMS vehicle icons at the interactive graphical map based on the real-time EMS vehicle location tracking information, each EMS vehicle icon representing a respective EMS vehicle for the EMS agency, and automatically capture the item of EMS trip information based on a user selection of at least EMS vehicle icon. The number of icons may include at least one post location icon and at least one individual EMS vehicle location icon, and the processor-readable instructions may be configured to cause the at least one processor to provide the at least one post location icon with a first shape and a first color, and provide the at least one individual EMS vehicle location icon with a second shape and a second color, where the second shape is different from the first shape or the second color is different from the first color or a combination thereof. The processor-readable instructions may be configured to cause the at least one processor to display one or more of crew information or vehicle information in response to a user selection of a particular icon of the number of icons. The vehicle information may include a connectivity status representing one of availability of communications with the mobile computing device associated with the EMS agency vehicle or unavailability of communications with the mobile computing device associated with the EMS agency vehicle, where the EMS agency vehicle corresponds to the particular icon. The processor-readable instructions may be configured to cause the at least one processor to provide filter options for the interactive graphical map that enable a user to filter display of the number of icons to a particular type of EMS vehicle.

In some embodiments, the processor-readable instructions are configured to cause the at least one processor to display a log of the EMS trip, the log including a current status of an EMS agency vehicle corresponding to the EMS agency vehicle assignment, a time of the generation of the EMS agency vehicle assignment, and a time interval from the generation of the EMS agency vehicle assignment to a wheels rolling status. The log may include one or more of a time or time interval from the generation of the EMS agency vehicle assignment for an en route status, an at-scene status, a transporting status, or an at-destination status. The log of the EMS trip may include a time for a pre-scheduled patient transport. The log of the EMS trip may include one or more of a vehicle identification number, a vehicle service level, crew member identification, contact information for a requester of the EMS services, or a connectivity status representing communications availability or unavailability between the mobile computing device and the cloud-based CAD system. The at least one processor may be configured to interoperate with an in-vehicle dispatch system provided in the cloud-based distributed processing architecture and configured to provide a navigation and dispatch application at the mobile computing device associated with the EMS agency vehicle, and the connectivity status may register unavailability between the mobile computing device and the cloud-based CAD system prior to a crew member of the EMS agency vehicle logging into a navigation and dispatch application provided at the mobile computing device associated with the EMS agency vehicle. The log of the EMS trip may include a time stamp control, a cancel trip control, an unassign trip control, an add trip control, a return trip control, and an add leg control. The time stamp control, upon selection, may be configured to enable a user to enter a custom time stamp including a clock time and a text label, where the text label represents an action, status, or activity corresponding to the clock time. The processor-readable instructions may be configured to cause the at least one processor to display the log of the EMS trip in a scrollable window.

In some embodiments, the EMS agency is a first EMS agency at a first geographic location, the dispatch GUI is a first dispatch GUI, the system includes a database of EMS vehicles associated at least with the first EMS agency and a second EMS agency at a second geographic location, and the processor-readable instructions are configured to cause the at least one processor to identify an EMS vehicle associated with the second EMS agency that is available for the EMS trip based on at least one dispatch code, receive a patient location associated with the EMS trip, determine that the second geographic location is closer to the patient location than the first geographic location, transfer the pending EMS trip assignment from the first EMS agency to the second EMS agency, and provide a transfer notification for the EMS trip at a second dispatch GUI at a computing system located at the second EMS agency. The transfer notification may include an assignment of an EMS agency vehicle associated with the second EMS agency to the EMS trip. The transfer notification may include a request for the second EMS agency to allocate resources to the pending EMS trip assignment without an assignment of the EMS agency vehicle associated with the second EMS agency. The database of EMS vehicles may include stored permission information that enables the at least one processor to transfer the EMS trip from the first EMS agency to the second EMS agency. The stored permission information may include data access guidelines that identify one or more types of EMS trip data that are exchangeable between the first EMS agency and the second EMS agency. A computing system located at the second EMS agency and the computing system located at the first EMS agency may be separate uncoupled computing systems such that a data exchange between the first EMS agency and the second EMS agency only occurs via the cloud-based distributed processing architecture of the cloud-based CAD system. The processor-readable instructions may be configured to cause the at least one processor to convert EMS trip data from a first format or dispatch workflow associated with the first EMS agency to a second format or dispatch workflow associated with the second EMS agency.

In some embodiments, the processor-readable instructions are configured to cause the at least one processor to interoperate with an in-vehicle dispatch system provided in the cloud-based distributed processing architecture, where the in-vehicle dispatch system is configured to provide a navigation and dispatch application at the mobile computing device associated with the EMS agency vehicle, and the dispatch GUI is configured to receive the trip acceptance from a user entry to the navigation and dispatch application and provide the EMS agency vehicle assignment to the navigation and dispatch application.

The foregoing general description of the illustrative implementations and the following detailed description thereof are merely exemplary aspects of the teachings of this disclosure and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A is a block diagram of a cloud-based software as a service platform and environment for managing computer-aided dispatching of EMS crews and vehicles;

FIG. 1B is a block diagram of a computer-aided dispatch system and environment for medical transport management across multiple EMS agencies;

FIG. 1C is a block diagram of a computer-aided dispatch system and environment supporting dispatch transfer between multiple EMS agencies;

FIG. 2 is a block diagram representing various services and data collection capabilities of a computer-aided dispatch system and environment;

FIG. 3A through FIG. 3E are screen shots of example user interface displays for managing EMS agency dispatch and assignment of EMS crews and patient transport vehicles;

FIG. 4A is a swim lane diagram of an example process for determining appropriate dispatch codes for dispatching EMS crews and patient transport vehicles;

FIG. 4B is a block diagram of an example system for determining appropriate dispatch codes for dispatching EMS crews and patient transport vehicles;

FIG. 5A and FIG. 5B illustrate a flow chart of an example method for determining appropriate dispatch services based on context and cues associated with a request for medical services;

FIG. 6A is a flow diagram of an example process for applying natural language processing to determining dispatch codes for dispatching EMS crews and transport vehicles;

FIG. 6B is a flow diagram of an example process for training machine learning algorithms to analyze caller responses and anticipate a shortest path of questioning to determine dispatch codes for dispatching EMS crews and transport vehicles;

FIG. 7A and FIG. 7B illustrate a flow chart of an example method for determining and coordinating appropriate staffing to respond to a medical assistance call;

FIG. 8 is a block diagram of an example cloud-based computer-aided dispatch system and environment for integrating telehealth services when responding to medical emergency calls;

FIG. 9 is a block diagram of an example information sharing architecture for sharing patient data and other information between a computer-aided dispatch system and other medical services and facilities;

FIG. 10A and FIG. 10B are a flow diagram of an example method illustrating an activation of response status through use of an in-vehicle dispatch system application; and

FIG. 11 illustrates a swim lane diagram of an example process for managing trip assignment among a computer-aided dispatch system, a mobile device of a patient transport vehicle, and a dispatch graphical user interface presented to a user.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration.

Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

The systems and methods described herein provide various advantages. In order to improve accuracy and streamline the dispatch process, the systems and methods described herein provide the advantage of a cloud-based CAD map-based automation in the dispatch data entry process.

The inventors recognized a need to improve emergency call triaging to determine an appropriate level of response along with the best services to deploy.

The inventors recognized a need to integrate non-dispatch and/or non-emergency services such as on-site paramedic care and/or telehealth professionals to provide support for those not requiring transport to a medical facility.

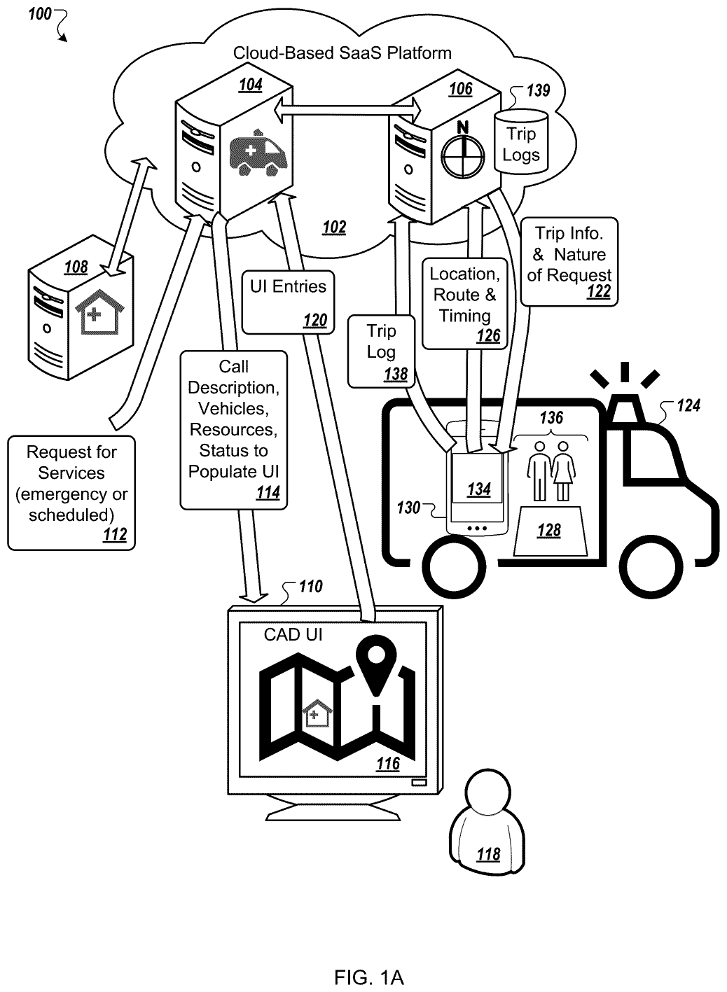

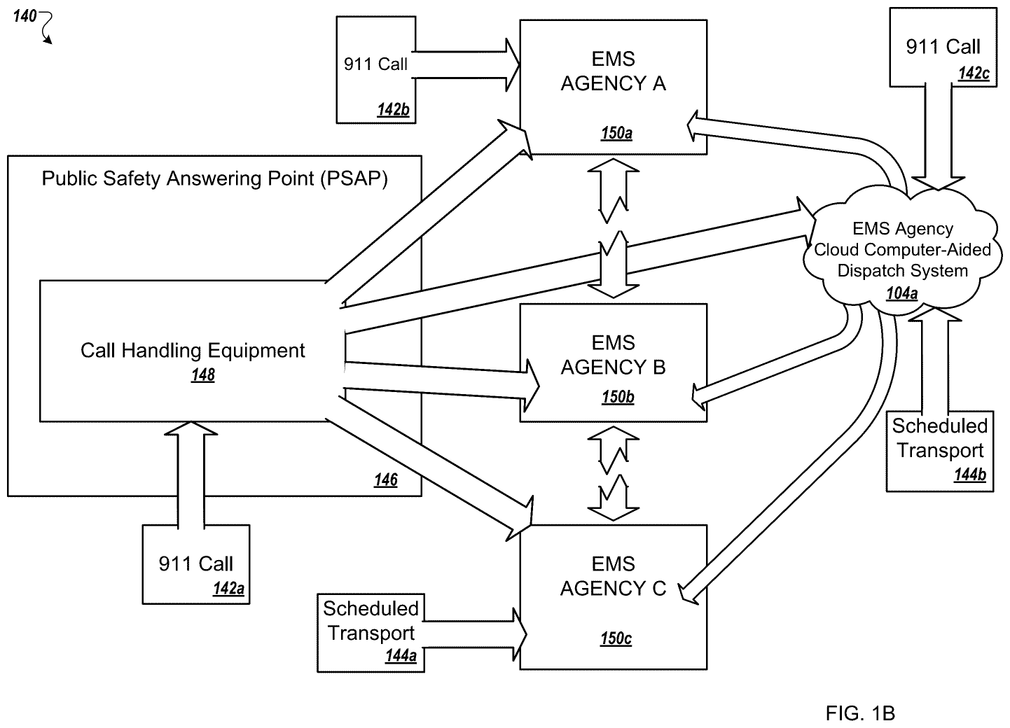

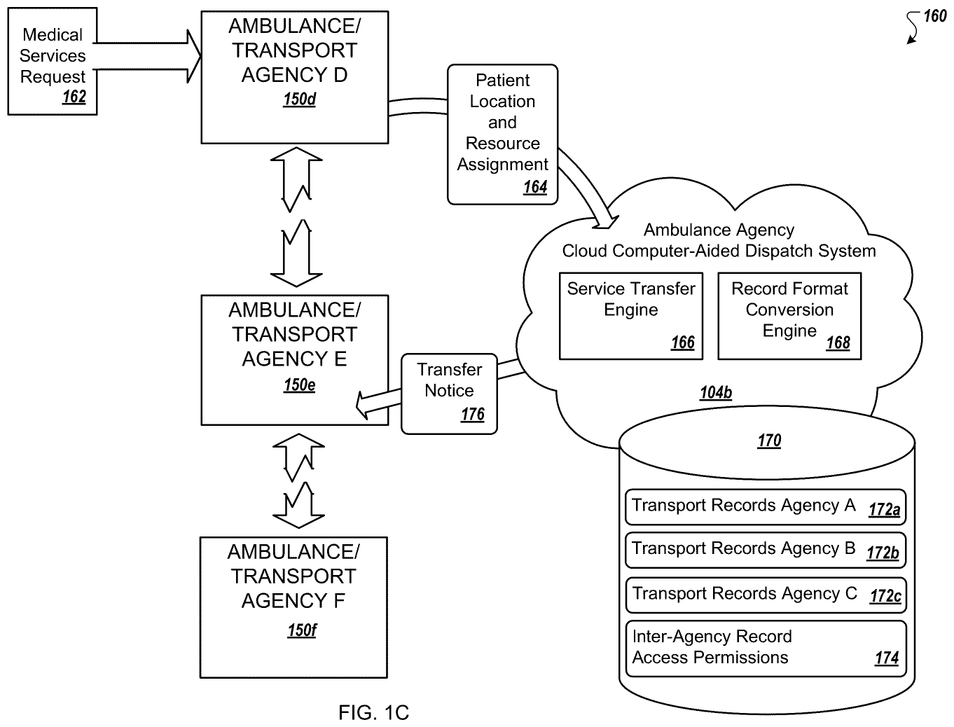

FIG. 1A is a block diagram of a cloud-based software as a service (SaaS) platform 102 and environment 100 for managing computer-aided dispatching of EMS crews and vehicles. As illustrated, the platform 102 includes two main service components, a computer-aided dispatch (CAD) system 104 and an in-vehicle dispatch system 106. The CAD system 104 is a medical dispatch system for dispatch of medical crews or EMS crews, and patient transport vehicles (e.g., EMS agency vehicle, emergency medical transport, etc.) in response to emergency calls and/or pre-scheduled patient transport requests. Although illustrated as separate systems 104, 106, in some embodiments, the CAD system 104 and the in-vehicle dispatch system 106 are integrated as a single system. In other embodiments, the CAD system 104 and the in-vehicle dispatch system 106 interoperate via at least one network connection. Additionally, although illustrated as being housed on separate computing platforms, each of the CAD system 104 and the in-vehicle dispatch system 106 may be implemented across a shared distributed cloud-based computing architecture, including the same and/or different processing resources (e.g., multi-processor computing units).

In some implementations, the in-vehicle dispatch system 106 provides a navigation and dispatch application 134. For example, a mobile device 134 such as a tablet, smartphone, and/or watch may implement the navigation and dispatch application 134 when the application 134 is stored in a memory of the mobile device 130 and/or accessed from a remote server or system (e.g., such as the cloud-based SaaS platform 102) via a network connection such as a cellular network and/or computer network. The mobile device 130 providing the application 134 may be associated with the emergency vehicle 124 assigned to a particular dispatch call and/or the crew 136 assigned to the particular dispatch call. The in-vehicle dispatch system 106 and the application 134 provided by the platform 102, in some embodiments, enables a transfer of dispatch information from the CAD UI 116 and the CAD system 104 to the crew 136. Similarly, the in-vehicle dispatch system 106 and the application 134 provided by the system 106, in some embodiments, enable a transfer of information from the crew 136 (e.g., via input to the application 134), and from a navigation sensor 128 associated with the emergency vehicle 124 (e.g., a global positioning system (GPS) device 128 disposed on the vehicle 124 and/or disposed on the mobile device 130 associated with and/or assigned to the vehicle 124 for a particular dispatch response). The CAD system 104 may receive vehicle location information from the mobile device, from a GPS device installed in or on the vehicle 124 and separate from the mobile device, or from a combination thereof. The information shared during the course of an assignment of an EMS trip (e.g., related to a particular dispatch call) may be captured by the application 134 in a trip log 138 (e.g., a time stamped list of activities and/or statuses compiled during a single EMS trip assignment) and shared with the in-vehicle dispatch system 106 and the CAD system 104 when the application 134 and mobile device 130 are communicatively coupled with the systems 104 and 106 (e.g., when network access permits transfer and/or sharing of the trip log 138). The trip log 138, in some examples, may include a vehicle identification number corresponding to the patient transport vehicle, a vehicle service level, crew member identification of one or more crew members, contact information for a requester of the EMS services, and/or a connectivity status representing a status of a communicative coupling between the computing device associated with the assigned patient transport vehicle and a monitoring system (e.g., CAD system 104). For example, the connectivity status may indicate a communications availability or unavailability (e.g., availability or unavailability of real-time or near real-time communications). Prior to a user of the navigation and dispatch application 134 initiating the application, for example by logging into the application 134 with user credentials, the connectivity status may indicate an unavailable communications link between the mobile device 130 providing the application 134 and the CAD system 104. The trip log 138 may be stored to a trip log data store 139. The CAD UI 116 and/or the mobile device 134 may provide the trip log 138 for use and review by a dispatcher and/or a crew member. The trip log 138, for example, may be presented in a scrollable window and may provide historical information for a trip, real-time information for a trip, or a combination thereof. The application 134 may update the trip log 138 in real-time as events of a trip occur.

In some implementations, the in-vehicle dispatch system 106 receives trip information 122 from the CAD system 104. The in-vehicle dispatch system 106 may provide the trip information to the application 134. The trip information 122 may include at least one destination (e.g., location of the patient, location of a medical facility for transporting the patient, name of a medical facility for transporting the patient, etc.) and a nature of the dispatch request. The trip information 122 may instruct a crew 136 assigned to the emergency vehicle 124 where to go and what to anticipate upon arrival. The nature of the request, for example, may include demographic information regarding the patient (e.g., name, gender, age, etc.) as well as details pertaining to the status of the patient and/or a medical status of the patient (e.g., unconscious, struggling to breathe, appears to be having heart problems, etc.) as known to the CAD system 104 through a request for services 112 (e.g., a request for EMS services).

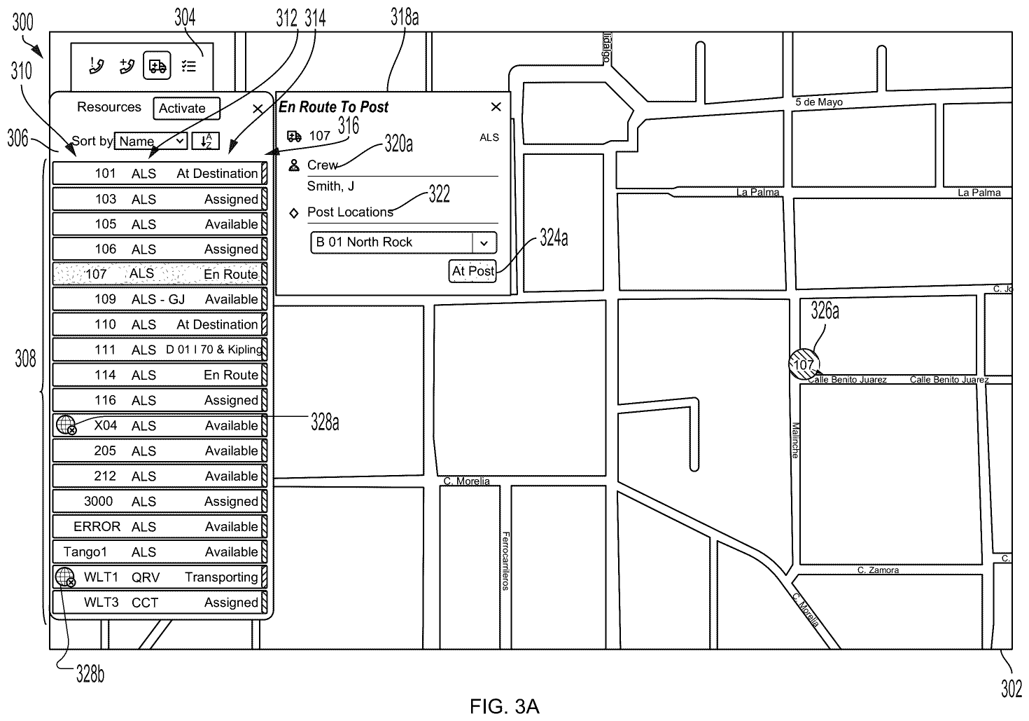

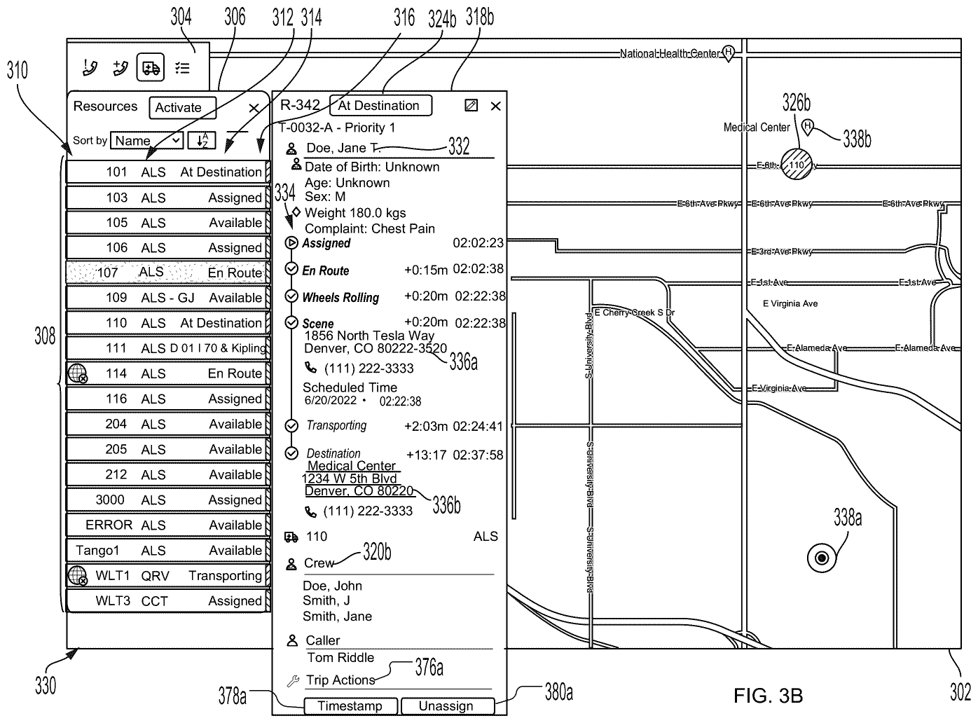

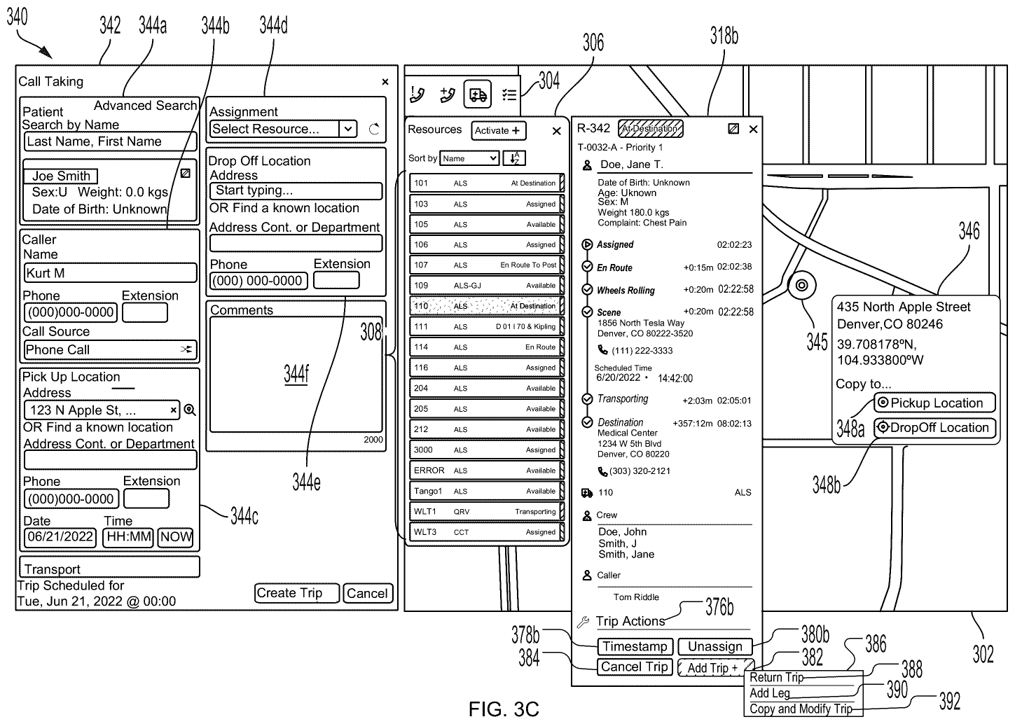

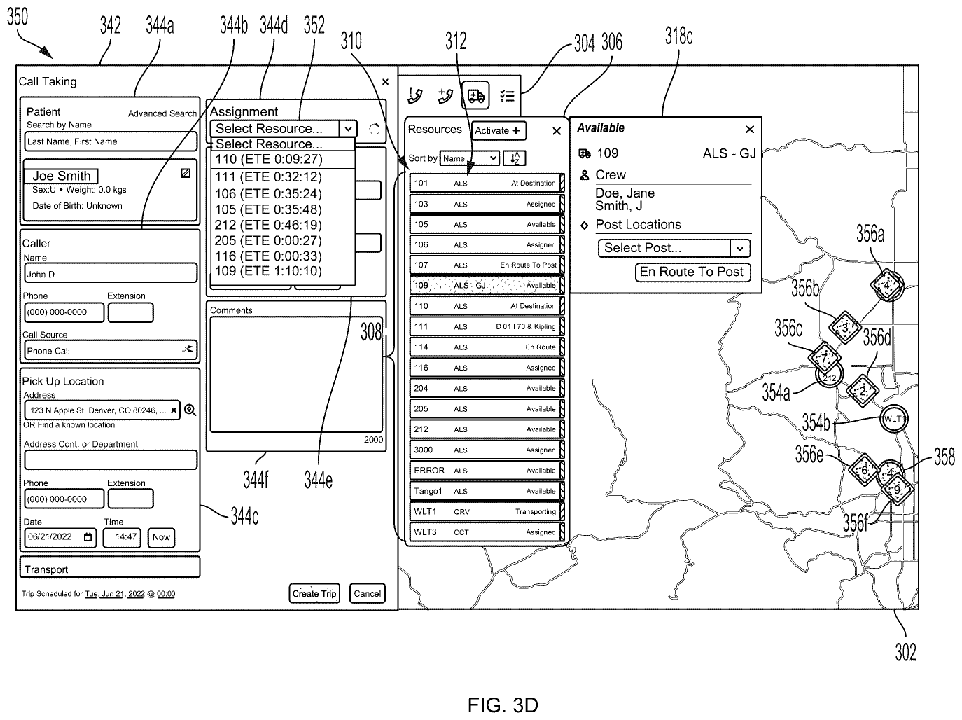

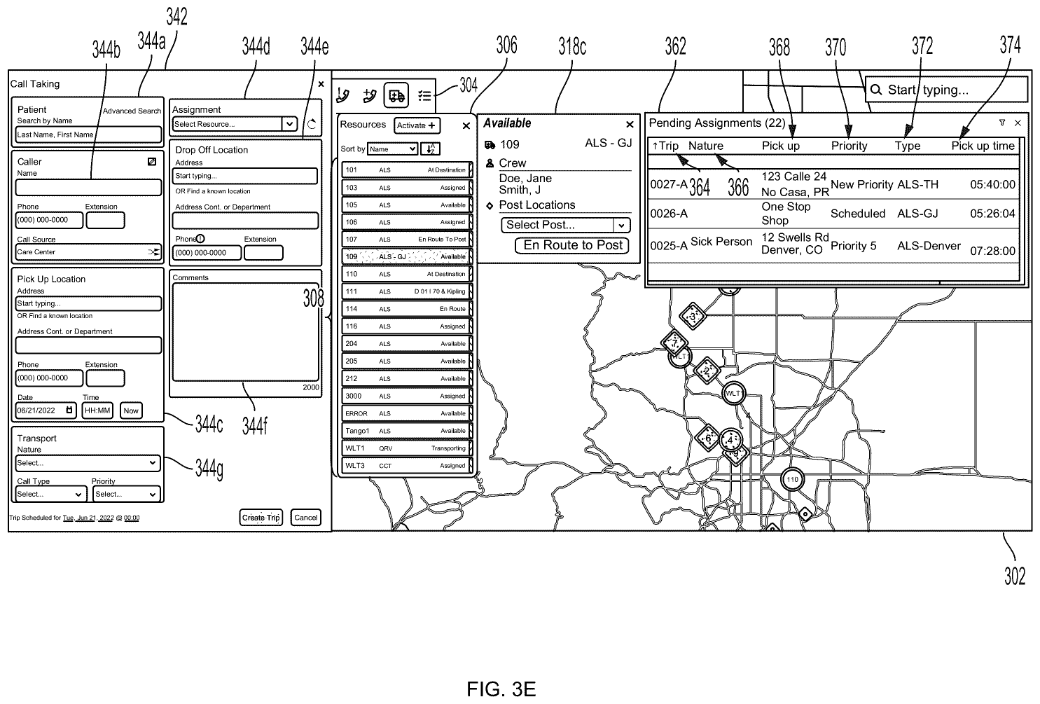

In some implementations, the application 134 communicates with the in-vehicle dispatch system 106 (and by extension the CAD system 104) during the course of the EMS trip. The application 134, for example, may provide, to the in-vehicle dispatch system 106, real-time or near real-time navigation information 126 including, for example, a current location of the emergency vehicle 124, a route (e.g., driving route) being taken by the emergency vehicle, and timing data (e.g., time of arrival at scene, duration of time at scene, departure time from scene, time of arrival at destination, etc.) related to the trip. The CAD system 104 may receive and utilize these automatically reported times to populate status fields (e.g., to automatically populate an item) in a listing of transport vehicle resources (e.g., the status fields 314 in the listing of transport vehicle resources 308 as shown for example in FIG. 3A) and/or to generate and populate a trip timeline (e.g., the trip timeline 334 as shown in FIG. 3B). The application 134 and/or the GPS sensor 128 may also automatically provide transport vehicle locations before, during, and/or after a dispatched call. This may enable the CAD system 104 to locate vehicles and display vehicle locations in real-time (e.g., the vehicle mapping information shown in FIGS. 3A-3E).

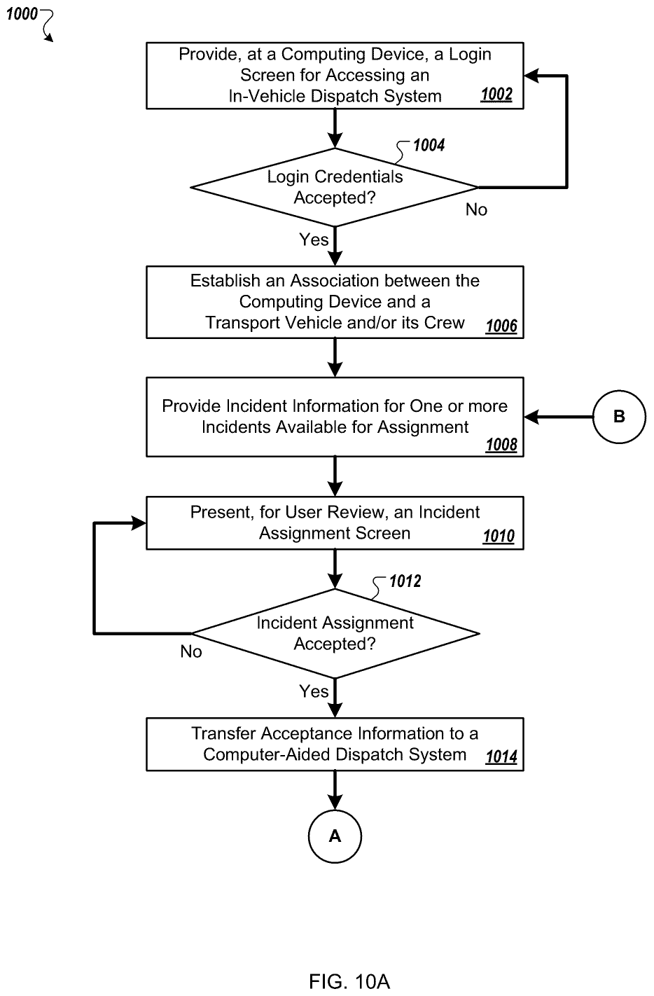

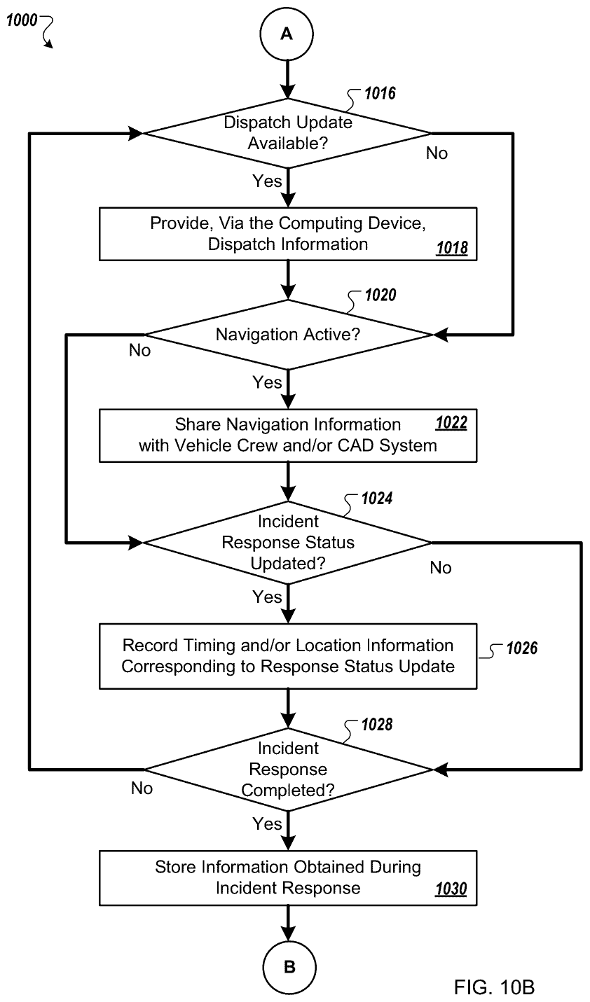

In some implementations, the application 134 implements response and call tracking features via a method 1000 as shown for example in FIG. 10A and FIG. 10B. FIG. 10A and FIG. 10B depict a flow diagram of an example method 1000 illustrating an activation of response status through use of an in-vehicle dispatch system application, such as the application 134 of FIG. 1A. The method 1000, for example, may be performed in relation to a trip scheduled by a computer-aided dispatch (CAD) system for an emergency medical vehicle.

Turning to FIG. 10A, in some implementations, the method 1000 begins with providing, at a display of a computing device (e.g., the mobile device 130 shown in FIG. 1A) for review by a user, a logic screen (1002). A user, for example, may log into the in-vehicle dispatch system application by providing information about the individual and/or entering authentication or credential information. At the login screen, the user may input, select, or otherwise specify information such as specific EMS company, username, and/or password.

Once login credentials have been accepted (1004), in some implementations, an association is established between the computing device presenting the login screen and a transport vehicle (1006). This login information, for example, may enable the systems 104 and 106 of FIG. 1A to associate a particular mobile device with a particular emergency transport vehicle and crew. The association, for example, may include selection of crew and vehicle identification (e.g., a vehicle identifier, vehicle identification number, etc.) at the display of the computing device by a user logging into the application 134. In another example, the systems 104 and/or 106 of FIG. 1A may use a GPS location of the mobile device 130 and/or of the location sensor 128 to reference the present location of a particular vehicle and crew. In a further example, the computing device and/or the location sensor 128 may be administratively dedicated to or physically associated with the particular emergency transport vehicle, such that the association between the computing device and transport vehicle is automatic, while the association with the crew may be derived (e.g., through additional user interface data entry and/or through the CAD system).

In some implementations, after the login credentials have been accepted (1004), the CAD system 104 may provide incident information for one or more incidents available for assignment to the transport vehicle 124 to the in-vehicle dispatch system 106 for display at the mobile device 130 via the application 134 (1008).

In some implementations, the application 134 presents an incident assignment user interface (UI) screen for user review (1010). The incident assignment UI screen may include one or more available assignments for user consideration. For example, following login at the computing device 130, the application 134 of FIG. 1A may provide, at the user interface screen (e.g., on the display region of the mobile device 130), an incident assignment and/or a list of available incident assignments. The incident assignment information may list, in some examples, the nature of each incident, the location of each incident, and/or a time that has elapsed since the incident assignment information was presented to the user (e.g., time elapsed since the CAD system 104 of FIG. 1A provided the incident information to the application 134). The incident assignment user interface screen may include one or more controls for accepting a given assignment of the one or more available incident assignments.

In some implementations, the CAD system 104 determines whether an incident assignment has been accepted at the computing device (1012) for a particular transport vehicle 124. A transport vehicle crew member (e.g., EMS services crew member) may accept or select a particular assignment, for example, via input to the application 134 (e.g., a tap or other touchscreen gesture for a touchscreen display and/or a typed input to a query field). If not already captured by establishing the association between the computing device and the transport vehicle (1006) as an automatic association at login based on crew member credential, the user may further specify the vehicle, unit, and/or additional crew members associated with the accepted assignment. During a time period where incidents are offered for acceptance, the list of incident assignment options may refresh or update according to new incidents and/or incidents accepted by other crews of other emergency medical vehicles.

In some implementations, acceptance information is transferred to the CAD system responsive to assignment acceptance (1014). The assignment information, for example, may be transferred to the CAD system 104 of FIG. 1A automatically via the in-vehicle dispatch system 106.

Transferring information between the computing device 130 associated with the transport vehicle 124 and crew 136 and the in-vehicle dispatch system 106 may depend on continuing network access (e.g., cellular/Wi-Fi, etc.) between the computing device 130 or other network access point and the in-vehicle dispatch system 106. A connectivity status of a communicative coupling between the computing device 130 and the SaaS platform 102 may be presented, for example, by the application 134 of the computing device 130 and/or the CAD UI 116, identifying the availability of real-time communication at a particular instance of time. The communicative coupling may be active or inactive based on, for example, network availability, a communication setting or status of the computing device 130 and/or the application 134, a communication setting or status of the CAD system 104, a communication setting or status of the in-vehicle dispatch system 106, etc. The connectivity status, for example, may depend on the in-vehicle dispatch system 106 successfully pinging the mobile device 130 via the application 134 (e.g., sending and receiving handshake connectivity information to establish or maintain a communicative coupling).

Turning to FIG. 10B, at least during periods of network availability, in some implementations, as dispatch updates from the CAD system become available to the computing device (1016), dispatch information is provided to the crew of the emergency vehicle (1018). Over the course of the dispatched call, for example, the application 134 of FIG. 1A may provide further dispatch information received at the CAD system 104. The dispatch information, in some examples, may include further incident details and/or an updated incident location presented to the user interface via the computing device. In another example, the dispatch information may include audio and/or video communications provided by dispatch personnel.

If in-vehicle navigation is active (1020), in some implementations, navigation information, such as maps, routing information, traffic information, and/or weather information, is presented to the crew and/or provided to the CAD system (1022). For example, the application 134 may provide in-vehicle navigation information to the crew obtained from a navigation app executing on the computing device 130 or another computing device in communication with the computing device 130. The user interface, in some embodiments, provides one or more interactive controls to the crew members, for example to toggle alternative map views (e.g., between zooming to the destination location, viewing the entire route (e.g., driving route), and following the vehicle on the map). Further, in some embodiments, a portion of the navigation information is provided, by the computing device 130, to the platform 102 for tracking the vehicle at the CAD 104. For example, the CAD UI 110 may be updated based on navigation information provided by the application 134.

In some implementations, if the incident response status has been updated (1024), timing and/or location information corresponding to the response status update is recorded (1026). For example, the incident response status may begin with assigned upon acceptance of the assignment. Once the vehicle begins traveling to the site of the incident, the incident response status may update to “en route” or “wheels rolling,” and the corresponding time at which the vehicle began traveling may be logged. Certain status updates may be automatically received by the computing device, such as navigation software indicating that the vehicle is en route or that the vehicle has arrived at the destination. Certain status updates may be manually entered by a crew member at the user interface of the computing device. The status, along with any recorded timing and/or location information, in some embodiments, is relayed to the CAD system (e.g., CAD system 104 of FIG. 1A). In some embodiments, the user interface includes the option for crew entry of incident time stamps, alerts, and/or other information associated with the in-progress response, such as notes.

As the incident response is ongoing, the dispatch information updates (1018), navigation information sharing (1022), and/or incident status updates (1026) may continue to be processed by the computing device in accordance with availability of new information.

In some implementations, once the incident response has completed (1028), information obtained during the incident response is stored (1030). An incident log, for example, may include information entered by crew members as well as information received from one or more external systems (e.g., the CAD system, the in-vehicle dispatch system, etc.). The log may be stored locally to the computing device (e.g., as the trip log 138 of FIG. 1A) and/or remotely to an element of the SaaS portal (e.g., the trip log data store 139 of FIG. 1A). For example, at the conclusion of a dispatched call, the application 134 of FIG. 1A may store all or a portion of the information entered and received during the call in a file stored at the system 104 and/or 106.

In some implementations, upon completion of the incident response, the method 1000 returns to presenting the incident assignment screen (1010) to offer additional assignments to the crew of the medical emergency vehicle. The user may be presented with the opportunity to remain logged in, in one example, prior to returning to the incident assignment screen. In other implementations, the user may be automatically logged out, and the method 1000 return to providing the login screen (1002).

Although described in relation to a particular set of operations, in other implementations, the method 1000 may include more or fewer operations. For example, in some embodiments, rather than just being provided with dispatch information (1018), the crew is enabled to communicate directly with a dispatch center using video, images, a voice call, and/or text massages. This communication may be bi-directional. For example, the mobile device 130 of FIG. 1A may be configured to both receive video, images, voice data, and/or text data from dispatchers via the CAD system 104 and send video, images, voice data, and/or text data to dispatchers. In this manner, in an illustrative example, the CAD system 104 may forward video, images, voice, and/or text information received from the scene (e.g., via the request for services 112) to the crew on route to the scene. In further embodiments, the crew may be enabled to communicate with the in-vehicle dispatch system interface associated with another crew and transport vehicle, for example to coordinate efforts in relation to a larger incident involving multiple patients. The in-vehicle dispatch system 106 of FIG. 1A, for example, may coordinate communications (e.g., video, images, voice, text, etc.) between crews of multiple transport vehicles.

Additionally, although depicted as a particular series of operations, in other implementations, certain operations of the method 1000 may be performed in a different order and/or at least partially concurrently. For example, in some implementations, in response to the acceptance of a particular assignment (1012), the mobile device and at least one crew member associated with the mobile device may be associated with the transport vehicle (1006). Further to the example, although multiple crew members are typically associated with a single transport vehicle and would therefore be receiving information from a single mobile device (e.g., device 130 of FIG. 1A), authentication and/or other login protocols may only allow a single crew member to activate the application (1004). In illustration, the mobile device may only support storage of a single face for a facial recognition authentication. In such a situation, the association of the mobile device with multiple crew members may occur via information provided to the application (e.g., application 134) and/or to the CAD system 104 outside of initial authentication information. Other modifications of the method 1000 are possible.

Returning to FIG. 1A, in some implementations, the CAD system 104 provides CAD services to one or more hospital systems 108 and/or emergency medical services (EMS) agencies via a CAD platform 110. The CAD platform 110, in some embodiments, is available via a long-range communications network by interfacing with one or more application programming interfaces (APIs). For example, a desktop or browser-based application may be configured to interoperate with the online CAD platform 110 through communicating via the APIs. The hospital systems 108, for example, may connect with the CAD system 104 via the long-range communications network to request transfer assistance to transfer patients between medical facilities and/or out of the hospital system. The CAD system 104, in turn, may populate a portion of a local user interface at the hospital systems 108 and/or the CAD platform 110 by supplying real-time and/or near real-time information regarding available resources, trips, patients, and/or navigation circumstances (e.g., to automatically populate an item of EMS trip information). The EMS agencies may obtain requests from the hospitals 108 and/or other external users (e.g., 9-1-1 emergency calls, non-emergency medical assistance requests, non-emergency patient transfer, etc.) by connecting with the CAD system 104 via the long-range communications network. Further, the long-range communications network connection between the EMS agencies and the CAD system 104 may enable the EMS agencies to respond efficiently and intelligently to the requests from the hospitals 108 and/or other external users by using real-time resource information and/or navigation information (e.g., supplied by the in-vehicle dispatch system 106) to fulfill incoming requests. The CAD system 104 may handle dispatch assignments of crews and patient transport vehicles in response to requests from the hospitals 108 and/or other external users such as 9-1-1 callers. The dispatch assignment may include a request to an EMS agency to dispatch a particular crew and/or patient transport vehicle as a trip assignment in response to the requests received by the CAD system.

In some implementations, the request for services 112 is received by the SaaS platform 102 (e.g., an emergency medical service request, non-emergency medical service request, and/or scheduled transportation request). The request may include, in some examples, patient demographic and/or medical information, patient location information regarding the patient location (e.g., address, GPS coordinates, name of a building, cross-streets, type of terrain, etc.), destination information, and/or time indication(s) (e.g., time of pick-up, time of drop-off). The request for services, for example, may be received at the CAD system 104 and assigned to a selected agency via the online CAD platform 110.

An agency operator logged into the online CAD platform 110, in some implementations, receives request information 114 at a CAD user interface including, in some examples, a call description, a set of vehicles, a set of personnel resources, and/or a set of medical equipment resources. The information, for example, may be used to populate a CAD user interface (UI) 116 (e.g., to automatically populate an item of EMS trip information). In some embodiments, portions of the information contain status designations. For example, each vehicle may be associated with a status such as, in some examples, available, assigned, wheels rolling, en route, at-scene, transporting, and/or at-designation. Similarly, each personnel and/or medical device resource may be associated with a status indicating availability and/or location.

In some implementations, an agency representative 118 interacts with the CAD UI, inputting UI entries 120 related to, in some examples, a patient, a pick-up location, a drop-off location, a nature of the request, and/or an assignment of vehicle, medical resources, and/or personnel resources in response to the request for services 112.

In some implementations, location information, such as pick-up location and/or drop-off location, included in the request for services 112 and/or the UI entries 120 are provided to the in-vehicle dispatch system 106 for working out route information related to the vehicles. The route information, further, can be provided via the information 114 to the CAD UI 116 to assist the agency representative 118 in identifying a vehicle closest to the scene.

Although described in relation to emergency vehicle transport, in other embodiments, the cloud-based software as a service (SaaS) platform 102 and environment 100 may be used for scheduling other and/or additional medical services. For example, the CAD system 104 may be configured to assist in dispatching telehealth services, community intervention services, community-based outreach services, paramedic services, mental health services and/or social services. The nature of the request, for example, may assist in identifying the appropriate service(s) and/or professional(s) to dispatch.

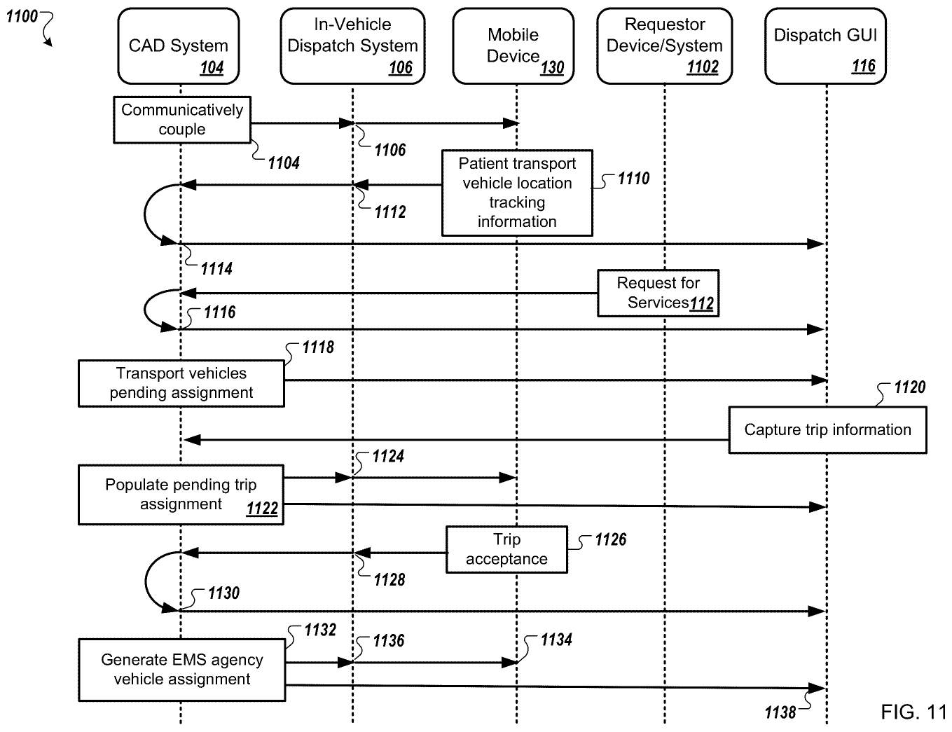

Turning to FIG. 11, a swim lane diagram of an example process 1100 for managing trip assignment among the computer-aided dispatch system 104, the mobile device 130, and the dispatch graphical user interface 116 is illustrated. The process 1100, for example, may be performed by the SaaS platform 102 and environment 100 of FIG. 1A.

In some implementations, the process 1100 begins with the CAD system 104 communicatively coupling (1104) with the mobile device 130. The communicative coupling, for example, may be established based on receiving and authenticating credentials provided by the mobile device 130. The authentication, in some examples, may be performed by the in-vehicle dispatch system 106 and/or the CAD system 104. In an illustrative example, the authenticating credentials may include a login identifier and password entered by a crew member at the mobile device 130. In another example, the authenticating credentials include an authentication token issued by the mobile device 130 to the in-vehicle dispatch system 106 upon gaining network access to the in-vehicle dispatch system 106 (e.g., when cellular and/or wireless network is within range of the mobile device 130). The coupling operation is routed (1106), in some embodiments, from the CAD system 104 to the mobile device 130 by the in-vehicle dispatch system 106. The in-vehicle dispatch system 106, for example, may maintain a secure communication link between the CAD system 104 and the mobile device 130.

In some implementations, location tracking information for a patient transport vehicle associated with the mobile device 130 (e.g., the patient transport vehicle 124 of FIG. 1A) is provided (1110) to the CAD system 104 by the mobile device 130. The location tracking information, for example, may include a geographic location (e.g., GPS coordinates), at least a portion of a route presently being followed by the patient transport vehicle (e.g., a route entered into a navigation system of the mobile device 130 and/or a navigation system of the patient transport vehicle), a present speed of the patient transport vehicle, and/or a present trajectory (e.g., north, south, east, west, or combinations thereof) of the patient transport vehicle. In some embodiments, the in-vehicle dispatch system 106 collects (1112) the patient transport vehicle location tracking information 1110 and forwards the patient transport vehicle location tracking information 1110 to the CAD system 104. In some embodiments, a secure communication link enabled by the in-vehicle dispatch system 106 is used to relay the patient transport vehicle location tracking information 1110 from the mobile device 130 to the CAD system 104. Although illustrated as a single transfer, the vehicle location tracking information may be provided repeatedly, for example on a periodic basis or as real-time EMS vehicle location tracking information while the patient transport vehicle is in motion.

In some implementations, the CAD system 104 provides (1114) at least a portion of the location tracking information for the patient transport vehicle to the dispatch GUI 116. The current position and/or indication of whether the patient transport vehicle is en route or otherwise in motion, for example, may be provided to the dispatch GUI 116 for presentation at a display device.

In some implementations, a requestor device or system 1102 provides the request for services 112 to the CAD system 104. The request for services 112, as described in relation to FIG. 1A, may be an emergency medical service request, a non-emergency medical service request, and/or a scheduled transportation request. The request for services 112 may be submitted, in some examples, via a mobile application, medical facility (e.g., hospital) portal application, emergency services (e.g., police, fire, etc.) dispatch equipment, and/or a telephone call. The request for services 112, in some examples, may include text, audio, and/or video content. The CAD system 104, in some embodiments, provides (1116) at least a portion of the content of the request for services 112 to the dispatch GUI 116 for presentation at a display.

In some implementations, the CAD system 104 provides information regarding one or more patient transport vehicles pending assignment 1118 to the dispatch GUI 116. The information regarding the one or more patient transport vehicles, for example, may include details such as those described in relation to the resource tab 306 of FIG. 3A.

In some implementations, trip information 1120 captured at the dispatch GUI 116 is provided to the CAD system 104. The captured trip information 1120 may include, in some examples, demographic information regarding a patient, symptom information regarding the patient, scene information regarding a surroundings of the patient, destination information regarding a location to transport the patient to, and/or priority information regarding the criticality of transport for the patient. The captured trip information 1120, for example, may include at least a portion of the information presented in a data entry pane 342 of an example graphical user interface 340 of FIG. 3C. Further, the captured trip information 1120 may include selection of one or more of the transport vehicles pending assignment 1118. For example, a particular type of patient transport vehicle, a vicinity (e.g., minutes to arrival, miles to arrival, etc.) of the patient, and/or a selected patient transport vehicle may be identified in the captured trip information 1120. In some embodiments, the CAD system 104 adds the captured trip information 1120 to a log. Although illustrated as being a single transfer, portions of the captured trip information 1120 may be provided to the CAD system 104 as entered into the dispatch GUI 116. The CAD system 104 may collect the captured trip information 1120 as a pending trip assignment record.

In some implementations, the CAD system 104 populates, for review at the mobile device 130, a pending trip assignment 1122. The pending trip assignment may include, for example, all or a portion of information provided in the request for services 112, the information 114, the UI entries 120, the trip information 122, etc. The pending trip assignment 130, for example, may be provided to the mobile device 130 for review by one or more crew members. In some embodiments, the CAD system 104 provides (1124) the populated pending trip assignment information 1122 to the in-vehicle dispatch system 106 for preparation in a format appropriate to rendering in a display of the mobile device 130.

In some implementations, the CAD system 104 provides at least a portion of the pending trip assignment information to the dispatch GUI 116. For example, the CAD system 104 may provide an indication of each transport vehicles of the transport vehicles pending assignment 1118 that successfully received information regarding the pending assignment.

In some implementations, the mobile device 130, responsive to receiving the pending trip assignment information 1122, issues trip acceptance information 1126 to the CAD system 104 indicating acceptance of the pending trip assignment 1122. For example, the CAD system 104 may provide the pending trip assignment 1118 to the navigation and dispatch application 134 provided at the mobile device 130 (e.g., via the system 106) and a user of the application 134 may enter an acceptance of the trip to the application 134. The trip acceptance information 1126 may include this user entry. In some implementations, the trip acceptance information 1126 may include additional information gathered by or available to the application 134. For example, this additional information regarding the trip may include identification of one or more crew members of the patient transport vehicle, an estimated time of arrival to the site of the patient, and/or an estimated departure time for a trip to reach the patient. Further, the trip acceptance information 1126 may include one or more requests for additional information, such as, in some examples, details regarding the patient, the route, the destination, and/or medical equipment required for the trip. The trip acceptance information 1126, in some embodiments, is forwarded (1128) to the CAD system 104 by the in-vehicle dispatch system 106. The in-vehicle dispatch system 106, for example, may translate information entered at the mobile device 130 into information recognized by the CAD system 104. The CAD system 104 may store the trip acceptance information 1126 to the log.

In some implementations, the CAD system 104 provides (1130) an indication of trip acceptance to the dispatch GUI 116. The trip acceptance, for example, may be presented at a display for review by a dispatcher.

In some implementations, the CAD system 104 generates an EMS agency vehicle assignment 1132. The EMS agency vehicle assignment 1132, for example, may include remittance and/or billing coding information, transport vehicle information (e.g., vehicle type, vehicle capabilities, vehicle inventory, etc.), crew identification information, and/or originating agency information (e.g., in the circumstance of agency transfer of the trip). Further, the EMS vehicle assignment 1132 may include at least a portion of the trip information 1120 obtained from the dispatch GUI 116 (e.g., all or a portion of information provided in the request for services 112, the information 114, the UI entries 120, the trip information 122, etc.). In some implementations, the pending trip assignment 1122 may include less information than the vehicle assignment 1132. For example, the pending trip assignment 1122 may include a smaller amount of information such as a location and nature of incident in order to enable a crew to make a trip acceptance decision and then the vehicle assignment 1132 may include more details to enable the crew to prepare for and provide the necessary emergency care. These further details may include more information about the location and nature of incident, may include information received at the CAD 106 from the emergency scene in the interim between the pending trip assignment 1122 and the vehicle assignment 1132. The CAD system 104, in some embodiments, provides 1134 the EMS vehicle assignment 1132 to the mobile device 130. For example, the CAD system 104 may provide the EMS vehicle assignment 1132 to the navigation and dispatch application 134. This provision may trigger data collection for the trip by the application 134 and/or the system 106. Additionally, this provision may initiate a timeline associated with the trip log so that time stamps indicate an interval between a confirmed assignment of the trip to the vehicle and actions associated with the trip. The EMS agency vehicle assignment 1132 may be provided (1136) via the in-vehicle dispatch system 106. The in-vehicle dispatch system 106 may store at least a portion of the EMS agency vehicle assignment, for example to an assignment log. In some embodiments, the CAD system 104 provides (1138) the EMS agency vehicle assignment 1132 to the dispatch GUI 116. The dispatch GUI 116 may present a portion of the EMS agency vehicle assignment 1132 information at a display for review by a dispatcher.

FIG. 1B is a block diagram of a CAD system 104a and environment 140 for managing dispatch assignments across multiple EMS agencies 150a-c, each including its own CAD platform 110 (see FIG. 1A). The dispatch assignments may include responses to emergency medical requests 142a-c (e.g., 911 calls, etc.) as well as scheduled patient transport requests 144a,b. In either case, the dispatch assignment is for an EMS crew and a patient transport vehicle such as an ambulance or helicopter. As illustrated, three EMS agencies 150a-c are illustrated. However, any number of EMS agencies may be connected to the CAD system 104a. The EMS agencies 150a-c, in some examples, can include agencies in a certain vicinity, such as a greater metropolitan area. In some embodiments, one or more of the EMS agencies 150a-c may be a subsidiary of a larger EMS agency (e.g., providing services to the greater metropolitan area).

In some implementations, an emergency medical request 142a is received by call handling equipment 148 of a public safety answering point (PSAP) 146. In illustration, a patient, caregiver, or bystander may issue a 911 call that is routed by the call handling equipment 148 to an appropriate PSAP dispatcher who manages the incoming request. The PSAP dispatcher may obtain initial information regarding the patient and/or the nature of the emergency to determine which EMS agency 150a-c to transfer the medical emergency to.

In some implementations, the PSAP dispatcher is assisted in the transfer decision through communications with the CAD system 104a. For example, similar to the CAD UI 116 of the EMS agency CAD platform 110 of FIG. 1A, the PSAP dispatcher may be provided real-time or near real-time information regarding available resources and/or estimated time of arrival relate to each of the EMS agencies 150a-c. The call handling equipment 148 of the PSAP 146, in some embodiments, supports transfer of the emergency medical request 142a (e.g., data transfer of the initial information captured by the PSAP dispatcher) to each of the EMS agencies 150a-c. In some embodiments, the CAD system 104a is configured to enable hand-off between the call handling equipment 148 and a selected EMS agency 150a-c.

In some implementations, the CAD system 104a is configured to directly receive an emergency medical request 142c and/or a scheduled transport request 144b on behalf of the connected agencies 150a-c and route the request 142c, 144b to the appropriate agency 150a-c. Although the request 142 is marked as a 911 call, in other examples, the request may be submitted by a police unit, a firefighting unit, and/or other emergency medical services/community services. The request 142, in addition to being submitted via a voice call (e.g., mobile device, cellular phone, shortwave radio, etc.) may be transmitted by computer or facsimile. The routing, for example, may be performed at least in part based on a location of the caller (e.g., derived through conversation and/or captured from GPS coordinates related to a mobile device used by the caller). Further, the routing may be performed in part based on availability of resources for each agency 150a-c, as determined by the transport CAD system 104a. In some embodiments, the routing is automated. For example, the initial information, such as location of the emergency, may be determined through natural language processing (NLP) if not capable of being derived from mobile device geocoordinates.

In some implementations, at least a portion of the EMS agencies 150a-c are configured to directly receive an emergency medical request 142b and/or a scheduled transport request 144a. As discussed in relation to FIG. 1A, for example, a dispatch agent at the agency 150a-c may receive resource information (e.g., available vehicles, available medical resources, available personnel resources, etc.) at a CAD UI (e.g., the CAD UI 116) to assist in scheduling emergency medical services related to the emergency medical request 142b or scheduled transport request 144a.

FIG. 1C is a block diagram of a CAD system 104b and environment 160 supporting dispatch assignment transfer between multiple EMS agencies 150d-150f. In the example of FIG. 1C, the various EMS agencies 150d-150f lack any business relationship that would enable these agencies to transfer calls between one another without the CAD system 104b. Each of the agencies 150d-150f lacks independent access to resource and dispatch information of any of the other agencies 150d-150f without the CAD system 104b. The computing system at a first EMS agency (e.g., agency 150d) and the computing system at a second EMS agency (e.g., agency 150e) are separate uncoupled computing systems. However, each of the agencies 150d-150f has a relationship with the CAD system 104b and provides the access permissions 174 that enable the CAD system 104b to reallocate dispatch calls amongst these agencies. Thus, data exchange between the first EMS agency and the second EMS agency only occurs via the CAD system 104b. To provide information such as vehicles, resources, and status information 114 to the EMS agency CAD platform 110 (see FIG. 1A), in some implementations, a transport CAD system 104b maintains, for each EMS agency 150d-150f, EMS agency records 172a-c. The EMS agency records 172a-c, in some examples, may detail the equipment and/or capabilities of various transport vehicles maintained by each agency 150d-f, demographic information, qualification/training information, and/or scheduling information regarding crew members for the transport vehicles, and/or status information for crew members and/or transport vehicles.

In some implementations, the transport CAD system 104b is configured to support sharing of at least a portion of the transport records 172a-b between a set of EMS agencies such as agency D 150d (e.g., at a first geographic location) and agency E 150e (e.g., at a second geographic location). The transport CAD system 104b, for example, may maintain inter-agency record access permissions 174 detailing which agencies are allowed access to each agency's records. Further, the inter-agency record access permissions may detail particular types of trip data (e.g., record fields) available for access and/or types of access available per type of record (e.g., read-only, read/write, etc.). For example, assuming agency E 150e is a subsidiary of agency D 150d, agency D 150d may be granted read/write access to certain records and/or fields of the agency E 150e transport records 172b, while agency E 150eB may be granted read-only access to certain records and/or fields of the agency D 150d transport records 172a.

Through record sharing, in some implementations, dispatchers at a first EMS agency 150d may select from both their agency's available resources as well as, if best suited, resources of a second EMS agency 150e. As shown in FIG. 1C, for example, a medical services request 162 received at EMS agency D 150d is assigned to a resource owned by EMS agency E 150e via a transfer assignment 164 including a patient location and the resource assignment (e.g., selected vehicle, etc.). In this manner, if a certain agency becomes overwhelmed, it may shift some medical emergency requests to a neighboring agency to address the needs of the greater community in a timely manner. In illustration, a catastrophic event such as a wildfire, flood, or earthquake may cause widespread injury in a particular geographic area, swiftly overwhelming the resources available to the dispatch agency nearest the location of the event. In sharing records, the overwhelmed agency 150a may trigger transfer of one or more incoming requests to the neighboring agency 150b.

In some implementations, a service transfer engine 166 enables transfer of the medical services request 162 from EMS agency D 150d to EMS agency E 150e. The service transfer engine 166, for example, may update the agency E 150e transport records 172b to reflect the resource assignment 164 and issue a transfer notice 176 for the attention of a dispatcher at a CAD UI of agency E 150e, such as the CAD UI 116 of FIG. 1A. In this manner, the dispatcher may follow the progress of the assigned trip.

The agencies configured to share information, in some embodiments, are separate and uncoupled computing systems such that record transfer is only available via the CAD system 104b. In other embodiments, such as a subsidiary set-up, a portion of the information may be transferred directly between the two agencies.

In some implementations, where two agencies sharing records have different record formats, the transport CAD system 104b includes a record format conversion engine 168 configured to convert the transport records 172 between different formats for sharing between agencies 150d-f (e.g., a conversion from a first format to a second format). The record format conversion engine 168, in some embodiments, converts the formatting of the agency E 150e transport records 172b when presenting agency E's resources for selection by a dispatcher at agency D 150d.

In some embodiments, the record format conversion engine 168 converts records from one dispatch workflow to another dispatch workflow (e.g., a conversion from a first dispatch workflow to a second dispatch workflow). In some examples, status indicators (e.g., pending assignment, assigned, etc.), links between data records, and/or priority notations may be converted by the record format conversion engine 168 to comport with the workflow of the second agency.

Although described in relation to an emergency request, in some implementations, inter-agency transfers include prescheduled medical transport. For example, a hospital may connect to a first agency to schedule transport, and that agency may assign the transport internally or to a subsidiary agency sharing records with the assigning agency. Other situations are possible, such as transfer of an in-progress trip due to mechanical failure of the initially assigned transport vehicle.

Although described in relation to a first agency assigning a particular transport vehicle belonging to a second agency, in some embodiments, the first agency may assign a trip generally to the transport fleet of a second agency. The transfer notification, for example, may include a request for the second agency to allocate resources to the trip as appropriate.

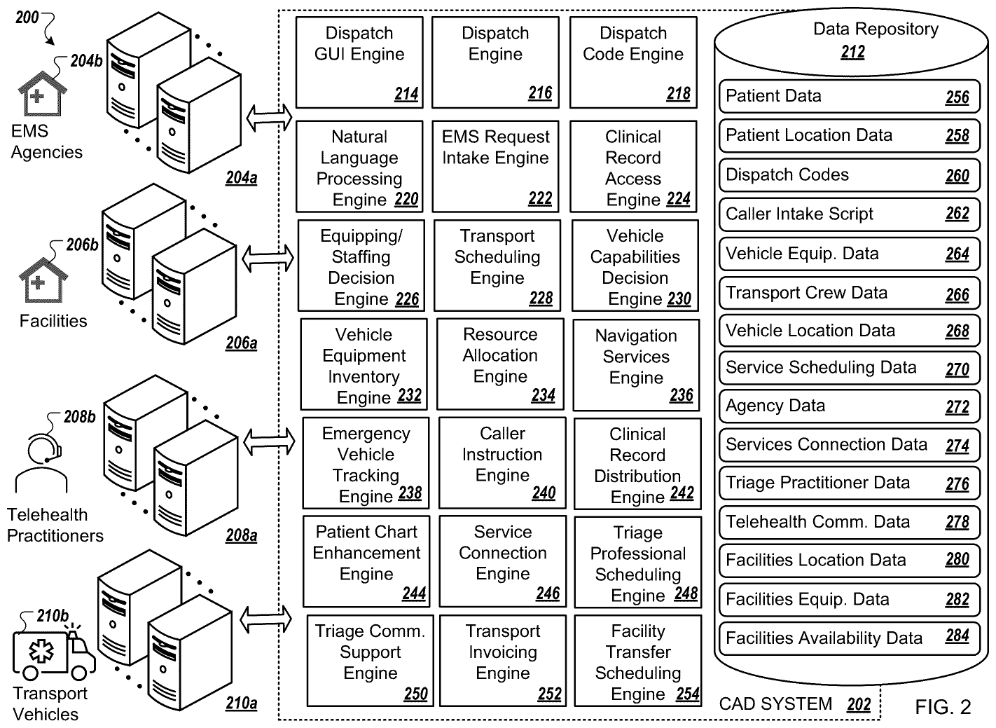

FIG. 2 is a block diagram representing various services and data collection capabilities of a computer-aided dispatch system 202 and environment 200. The CAD system 202, for example, may perform portions of the capabilities of the CAD system 104 and the in-vehicle dispatch system 106 of FIG. 1A, the CAD system 104a of FIG. 1B, and/or the CAD system 104b of FIG. 1C. The CAD system 202, for example, may be configured in the distributed processing architecture of a cloud computing environment. In some implementations, computing systems 204a, 206a, 208a, and 210a associated, respectively, with various EMS agencies 204b, healthcare facilities 206b, telehealth practitioners 208b, and/or patient transport vehicles 210b connect to the CAD system 202 via a set of application programming interfaces (APIs) configured to provide user interfaces and/or data appropriate to each type of user (e.g., agency, facility, telehealth, and/or transport vehicle crew). In some implementations, computing systems 204a, 206a, 208a, and 210a connect to the CAD system 202 via a communicative coupling via a cellular network and/or a computer network to exchange voice and/or video data.

In some implementations, the CAD system 202 includes a dispatch graphical user interface (GUI) engine 214 configured to display a graphical user interface to a user at one of the agencies 204b and/or facilities 206b via an associated computing system 204a and/or 206a, respectively. The dispatch GUI engine 214, for example, may present the CAD UI 116 of FIG. 1A. The dispatch GUI engine 214 may generate an interactive browser-based, portal-based, or web application-based user interface for collecting information related to a patient and enabling selection of services and/or resources to dispatch on behalf of the patient. For example, the dispatch GUI engine 214 may provide a web-based scheduling application for a computing system 206a of a medical facility 206b. The dispatch GUI engine 214, for example, may present the user interface screens described in relation to FIG. 3A through FIG. 3E, discussed below.

In some implementations, the dispatch GUI engine 214 provides an input/output (I/O) mechanism for engaging with a dispatch engine 216. The dispatch engine 216 performs and/or coordinates various operations for selecting and mobilizing services and/or resources.

In some implementations, the dispatch engine 216 collects and stores patient data 256 and patient location data 258 related to a medical services request. The patient data 256 and/or patient location data 258, for example, may be received from a connected user via the dispatch GUI engine 214.

In some implementations, the dispatch engine 216 launches an EMS request intake engine 222 to obtain the patient data 256 and/or the patient location data 258. The EMS request intake engine 222, for example, may cross-reference patient demographics entered via the dispatch GUI engine 214 with stored patient data 256 to supplement details regarding the individual. The EMS request intake engine 222, in another example, may derive geocoordinates of a caller using a mobile device or other global positioning system (GPS)-capable device to determine the patient location data 258 and/or refine the location of the patient (e.g., the caller may identify the convenience store on main street, and the GPS coordinates may be used to pinpoint the rear parking lot as the location).

In some implementations, the EMS request intake engine 222 uses a clinical record access engine 224 to obtain medical background information related to the patient. The clinical record access engine 224, for example, may use patient data 256 (e.g., name, birth date, home address, phone number, insurance identifier, etc.) to cross-reference clinical records to obtain medical history for the patient. The medical history, for example, may be used to guide initial recommendations and/or refine selection of services for dispatch in accordance with the patient's unique needs. In some examples, a heart condition, past surgeries, major diseases, and/or neurological disorder may all be helpful factors in analyzing the symptoms or condition of the patient.

In some implementations, the dispatch engine 216 launches a dispatch code engine 218 to determine one or more dispatch codes 260 related to a medical services request. The dispatch code engine 218 may be launched, for example, responsive to selection of a triage dialogue control presented in a dispatch GUI. In another example, the EMS request intake engine 222 may be configured to automatically initiate the dispatch code engine 218 upon creating a new trip corresponding to a medical emergency. The dispatch codes, for example, may be determinant codes, triage codes, and/or medical billing codes. A portion of the dispatch codes 260, for example, may relate to a health concern, injury, or physical state of the patient. Further, a portion of the dispatch codes 260 may relate to situational circumstances of a scene or surroundings of the patient (e.g., located in an area a vehicle can't reach, in the midst of a crime scene, area struck by natural disaster, etc.). Each dispatch code, in an illustrative example, may correspond to a medical condition descriptor and a priority indicator. The priority indicator, for example, may correlate to vehicle service levels or service capabilities of medical transport vehicles and/or EMS crews available and/or associated with particular vehicles.

In some implementations, the dispatch code engine 218 uses a caller intake script 262 (e.g., a series of questions) to obtain information regarding the medical request. The caller intake script 262, for example, may be presented to a dispatcher at an agency 204b via the associated computing system 204a by the dispatch GUI engine. Answers to questions posed using the caller intake script 262, for example, may provide the dispatch code engine 218 with information necessary to determine the dispatch codes 260 relevant to the medical services request. The series of questions may be stored by the EMS request intake engine 222.

In some implementations, the dispatch code engine 218 uses medical information obtained by the clinical record access engine 224 in determining the dispatch codes 260 to apply to the medical request. For example, certain vehicle equipment and/or capabilities may be necessary or preferable based on the patient's known medical issues.

In some implementations, the dispatch code engine 218 uses a natural language processing engine 220 to analyze caller responses to the caller intake script 262. The natural language processing engine 220, for example, may receive an audio stream or audio recording of caller responses and identify terms indicative of various dispatch codes 260.

In some implementations, using the dispatch codes 260, an equipping and/or staffing decision engine 226 determines the appropriate medical service(s) to dispatch to the patient. The equipping and/or staffing decision engine 226, for example, may identify transport crew certifications (e.g., paramedic qualifications, employee type), transport vehicle capabilities, transport vehicle medical equipment, a priority of response to the patient (e.g., critical, urgent, standard, etc.), a specialty or certification of telehealth provider, and/or other types of services (e.g., community intervention services, community-based outreach services, mental health services, social services, etc.) to dispatch to aid the patient. The equipping and/or staffing decision engine 226 may also use information provided by the clinical record access engine 224 to identify the equipping and/or staffing needs for the request.

In some implementations, the equipping and/or staffing decision engine 226 coordinates with a vehicle capabilities decision engine 230 to identify vehicle capabilities matching patient requirements. The vehicle capabilities, in some examples, may include real-time telehealth communication capabilities for enabling doctor interaction during transit, sizing or interoperability needs for a patient's medical equipment, and/or terrain capabilities based on patient location (e.g., tire chains for steep snow climb, etc.).

In some implementations, one or more engines of the CAD system 202 may interoperate with the in-vehicle dispatch system 106. For example, one or more of the engines 214, 216, 226, 228, 230, 232, 234, 236, 238 may interoperate with the in-vehicle dispatch system 106. In an implementation, one or more of the engines of the CAD system 202 may interoperate with the in-vehicle dispatch system 106 via the navigation services engine 236. For example, the navigation services engine 236 may communicatively couple with the in-vehicle dispatch system 106 to receive navigation, location, and/or trip timing information for a transport vehicle and/or to send navigation, location, and trip timing information for a transport vehicle. In some implementations, the navigation system 128 and/or the mobile device 130 may communicate with the CAD system 202 directly without intermediate communications with the in-vehicle dispatch system 106. In some implementations, the navigation system 128 and/or the mobile device 130 may communicate with the CAD system 202 via intermediate communications with the in-vehicle dispatch system 106. The CAD system 202 may utilize location information, navigation information, crew information, trip information etc. collected via the in-vehicle dispatch system 106 to inform various functions. In various implementations, the data repository 212 of the CAD system 202 may receive at least a portion of data from via the in-vehicle dispatch system 104. For example, the CAD system 202 may receive one or more of the patient data 256, the patient location data 258, the vehicle equipment data 264, the transport crew data 266, and/or the vehicle location data 268 via the in-vehicle dispatch system 104.

In some implementations, a vehicle equipment inventory engine 232 obtains vehicle equipment data 264 from the agencies 204 and maintains an inventory of vehicle features for use in dispatching the vehicle that best fits a patient's needs. The vehicle equipment inventory engine 232, for example, may classify vehicles according to a number of capability categories, such as communications features, medical equipment integration features, and/or terrain features (e.g., van/helicopter/boat/etc.). The vehicle equipment inventory engine 232, based on completed and/or en route trips, may further predict depletion of consumable vehicle features such as, in some examples, oxygen tank level and/or fuel level. For example, the vehicle equipment inventory engine 232 may anticipate that a vehicle cannot be dispatched on a new trip prior to stopping at a fueling station. In some implementations, the vehicle capabilities decision engine 230 may interoperate with the vehicle equipment inventory engine 232 to determine vehicle capabilities based on vehicles availability according to the inventory. The vehicle capabilities decision engine 230 may limit the capability decisions to vehicles actually ready and available for transport according to the inventory.

In some implementations, the equipping and/or staffing needs determined by the equipping/staffing decision engine 226 are provided to a transport scheduling engine 228 to schedule medical transport for the patient. The transport scheduling engine 228, for example, may identify appropriate vehicles to present for selection by a dispatcher at one of the agencies 204 and/or personnel at one of the facilities 206 in the event of a prescheduled transport on behalf of the medical facility.

In some implementations, the transport scheduling engine 228 determines, using vehicle equipment data 264, transport crew data 266, and/or vehicle location data 268, available medical transport vehicles for fulfilling the needs of a medical services request. The transport scheduling engine 228 may obtain a portion of the information from the vehicle equipment inventory engine. The transport scheduling engine 228, for example, may provide available vehicles for review by an end user via the dispatch GUI engine 214. Responsive to receipt of a selected transport, the transport scheduling engine 228 may provide scheduling information to a resource allocation engine 234 for allocating the resources.

In some implementations, a resource allocation engine 234 allocates transport crew, medical equipment, and/or services responsive to the patient's needs. The resource allocation engine 234, for example, may communicate with telehealth practitioners 208b (e.g., via the associated computing system 208a) and/or other service providers to involve the service providers in responding to the patient. The resource allocation engine 234, further, may update data in the data repository, such as service scheduling data 270 (e.g., services beyond those supplied by the agencies 204) and/or agency data 272 to identify resources allocated to the response.

In some implementations, the transport scheduling engine 228 coordinates with a navigation services engine 236 to determine an availability for dispatch for a set of transport vehicles. The availability for dispatch may include an estimated time of arrival of a particular vehicle at a patient scene. The navigation services engine 236, for example, may identify, using the vehicle location data 268, a current position of the set of transport vehicles. Using the patient location data 258, the navigation services engine 236 may calculate a driving route and driving time for each transport vehicle to reach the patient. The estimated time en route (ETE) may be determined, for example, based on the driving route. In some embodiments, the navigation services engine 236 receives the route and driving time information from the in-vehicle dispatch system 106.

In some implementations, during transit, an emergency vehicle tracking engine 238 generates and/or receives vehicle tracking information. For example, the engine 238 may track each transport vehicle 210 via a connection with the mobile device 130 and/or the navigation sensor 128 to generate vehicle tracking information. Alternatively or additionally, the engine 238 may receive tracking information from the mobile device 130 and/or the navigation sensor 128 via the in-vehicle dispatch system 106. The emergency vehicle tracking engine may store and/or update vehicle locations (e.g., in vehicle location data 268) and may maintain real-time EMS vehicle location and tracking information. The emergency vehicle tracking engine 238 may provide the real-time EMS vehicle location and tracking information for review by a dispatcher at the agency 204, via the dispatch GUI engine 214. The vehicle tracking information may include real-time mapping, traffic monitoring, identification of blockages or slowdowns, etc.

In some implementations, after obtaining information needed for dispatching services, the dispatch engine 216 launches a caller instruction engine 240 to provide the caller with advice and/or support in managing the state of the patient prior to the arrival of assistance. The caller instruction engine 240, for example, may present a script via the dispatch GUI engine 214 for the dispatcher to read to the caller. In another example, the caller instruction engine 240 may communicate with the caller via a voice engine and natural language processing of utterances of the caller. The caller instructions, in some examples, may provide simple first aid instructions, advice on an initial treatment (e.g., over the counter medication, anxiety-reducing activities, etc.), and/or instructions on receiving the medical services (e.g., turn outdoor lights on, unlock the door, etc.). Further, the caller instructions may instruct the caller in obtaining additional details regarding the patient's state which can be shared with the EMS crew. In illustration, the caller may be asked to time contractions of a pregnant patient or take a pulse of an incoherent patient.

In some implementations, after services have been dispatched, a clinical record distribution engine 242 supplies a portion of the patient's clinical records (e.g., obtained by the clinical record access engine 224) to one or more dispatched caregivers. The clinical record distribution engine 242, for example, may provide clinical health record data to one of the telehealth practitioners 208 and/or transport crew members of the transport vehicles 210. The clinical record distribution engine 242, for example, may enable secure, encrypted transfer in compliance with patient privacy requirements, such as Health Insurance Portability and Accountability Act (HIPAA) requirements.

In some implementations, the mobile device 134 provides an electronic patient care report (ePCR) application. The patient chart enhancement engine 244 may provide patient information obtained or received by the CAD system 202 to the ePCR application. In turn, the ePCR application may populate an ePCR with the patient information received from the CAD system 202. The patient information, for example, may include the patient data 256, clinical information corresponding to the assigned dispatch codes 260, and/or a portion of the patient medical records obtained by the clinical record access engine 224. The patient information may further include information provided to the CAD system 202 via the caller instruction engine 240. In some implementations, patient information provided to the ePCR application from the CAD system 202 may be initial information for the start of an EMS call involving the patient. This initial information may include, for example, one or more of demographic information (e.g., a patient first name, a patient surname, a social security number, an address, a gender, an age, etc.), provider information, insurance information, a chief complaint, emergency scene details, mechanism of injury, photographs, and/or video of the patient and/or the scene, or other information collected by the CAD system 202.

In some implementations, a service connection engine 246 connects the caller with an assigned service or practitioner, such as one of the telehealth practitioners 208 or mental health services. The assigned service or practitioner may be medical services unassociated with the transport vehicle 124 and/or unassociated with a dispatched EMS crew. Thus, the service connection engine 246 may enable provision of medical services without the dispatch of a crew and transport vehicle to a patient site. In some implementations, the CAD system 202 may invoke the service connection engine 246 in combination with dispatch of a crew and transport vehicle to the patient site. The service connection engine 246, for example, may automatically transfer the present call to a selected practitioner, for example using services connection data 274. In another example, the service connection engine 246 may text a link to the caller for launching a communication session (e.g., video call) with the assigned service provider.

In some implementations, the equipping/staffing decision engine 226 uses a triage professional scheduling engine 248 to schedule a telehealth practitioner 208 for virtual support of transport crew. The triage professional scheduling engine 248, for example, may access triage practitioner data to determine qualifications and availability matching the staffing requirements for the medical service request. The triage professional scheduling engine 248, for example, may provide scheduling information and connection details to a computing system 208a associated with a selected telehealth practitioner 208b to dispatch the telehealth practitioner 208b in support of the selected transport vehicle 210b.

In some implementations, dispatching a triage professional for monitoring and/or guiding transport crew involves enabling communication between the telehealth practitioner 208 and the transport vehicle 210. A triage communication support engine 250, for example, may establish a secure communication channel between one of the telehealth practitioners 208 and a videoconferencing system of one of the transport vehicles 210. The triage communication support engine 250, for example, may connect with the videoconferencing system using information stored in the telehealth communications data 278.

In some implementations, a transport invoicing engine 252 translates the allocated resources (e.g., professionals, equipment, transport vehicle, etc.), dispatch codes 260, and/or triage communication session into billing codes for invoicing purposes. The transport invoicing engine 252, for example, may communicate billing codes to a patient billing platform to streamline invoicing of medical services provided to the patient. In another example, the billing codes identified by the transport invoicing engine 252 may be used for insurance pre-approval of a prescheduled medical transport. If the request initiated with a medical facility, the billing codes may be provided to the facility 206 for inclusion in patient billing.

When the dispatch GUI engine 214 is being used by personnel at a medical facility 206, in some implementations, a facility transfer scheduling engine 254 assists in determining scheduling options for patient transfer between two of the facilities 206. The facility transfer scheduling engine 254, for example, may access facilities location data 280 to identify a set of closest medical facilities to the facility requiring transfer. To narrow the set of closest medical facilities, in some embodiments, the facility transfer scheduling engine 254 accesses facilities equipment data 282 to identify a subset of nearby facilities equipped to meet the patient's medical needs. The facility transfer scheduling engine 254 may provide a set of available times during which the receiving facility has availability for intake of the transferring patient. In addition, in some embodiments, the facility transfer scheduling engine 254 accesses facilities availability data 284 to identify open beds in an appropriate unit for the patient.

FIG. 3A through FIG. 3E are screen shots of example user interface displays for managing EMS agency dispatch and assignment of EMS crews and vehicles. The user interface displays, for example, may be presented to dispatchers at the agencies 204 of FIG. 2 by the dispatch GUI engine 214.

Turning to FIG. 3A, an example user interface display 300 includes a map 302 overlaid with a dispatch menu 304 having a resources tab 306 open. The resources tab 306 is populated with a listing of transport vehicle resources 308 (e.g., a listing of agency resource vehicles). Each transport vehicle is represented by an identifier 310 (e.g., name, vehicle number, etc.), one or more care level capabilities 312 (e.g., vehicle capabilities such as, in some examples, basic life support (BLS), advanced life support (ALS), ALS level 1 (ALS1), ALS level 2 (ALS2), specialty care transport (SCT), quick response vehicle (QRV), critical care transport (CCT), and various regional designations, such as ALS-GJ, with “GJ” being a regional designation for “Grand Junction, Colorado.” etc.), and a status 314 (e.g., available, assigned, en route, transporting, at destination, etc.). Each transport vehicle is also marked with a visual indicator 316 representing scheduling availability.

In some implementations, a connectivity indicator 328 is presented in relation to at least certain transport vehicles. For example, connectivity indicator 328a presented in relation to transport vehicle “X04” (identified as “available”) may indicate unavailability of communications with a computing device associated with the transport vehicle “X04” (e.g., that a computing device or application associated with transport vehicle “X04” is offline or that no crew member is logged into the application). In another example, a connectivity indicator 328b, presented in relation to transport vehicle “WLT1” which has a status of “transporting,” may indicate that the computing device associated with transport vehicle “WLT1” has lost network connection (e.g., no cellular or Wi-Fi service is available in the area the vehicle is presently traveling through). Conversely, in other embodiments, a connectivity indicator may be provided to represent availability of communications with a computing device associated with the transport vehicle.

In some implementations, a dispatcher may be provided with one or more filter options to filter the resources 308, for example by capability 312 and/or status 314. Alternatively, the dispatcher may arrange by capability 312 and/or status 314, thereby grouping resources 308 having a same capability 312 or a same status 314 together.

A pop-up window 318a corresponding to a selected transport vehicle 308 (e.g., vehicle 124 of FIG. 1A) is presented over a portion of the map 302. The pop-up window 318a, for example, displays agency resource information for the selected transport vehicle 308, corresponding to one of the location icons 324 presented on the map 302. The pop-up window 318a, for example, may be presented based on selection of a control for reviewing further information corresponding to the selected transport vehicle 308 (e.g., a clickable button). In the window 318a, crew member information 320a, a post location 322 (e.g., depot location housing medical transports when not deployed), and a current location 324a (e.g., “at post”) are presented. An agency resource location icon 326a overlaid on the map 302 provides a visual indication of the present location of the selected transport vehicle 308. In some embodiments, the pop-up window 318c presents or includes a control configured to present one or more pending assignments for the vehicle (e.g., if the status of the vehicle is listed as “assigned”).

Turning to a user interface 330 of FIG. 3B, a second transport vehicle 308 (e.g., vehicle 124 of FIG. 1A) is now selected. A pop-up window 318b corresponding to the second selected transport vehicle 308 includes crew member information 320b and a current location 324b (e.g., “at destination”), similar to the pop-up window 318a of FIG. 3A. Additionally, the pop-up window 318b includes patient information 332 (date of birth, age, sex, weight, complaint) and a timeline 334 of trip events. An agency resource location icon 326b overlaid on the map 302 provides a visual indication of the present location of the selected transport vehicle 308.

In some implementations, the timeline 334 illustrates, for each trip status (e.g., an assigned status, an en route, a wheels rolling status, an at scene status, a transporting status, an at destination status, etc.) a begin time as well as an elapsed time (e.g., a time interval from a former status to a latter status, such as a time interval from assignment to a wheels rolling status). For example, the assigned time is listed as 2:02:23, and the wheels rolling time is listed as 2:22:38, with a time interval between the assigned time and the wheels rolling time of 20 minutes. As illustrated, the timeline 334 may be presented in a scrolling (scrollable) window so that all of the trip details may be reviewed within the window.

In some embodiments, the pop-up window 318b includes a time stamp control for adjusting the presentation of timestamps in the timeline 334 (e.g., actual time only, elapsed time only, etc.). The pop-up window 318b may include a cancel trip control for cancelling the assigned trip. The cancel control, for example, may be available only during an appropriate period of time (e.g., prior to picking up the patient). The pop-up window 318b may include an unassign trip control. Using the unassign trip control, for example, may move the trip to a pending assignment list (described below in relation to FIG. 3E). The pop-up window 318b may include an add trip control to assign an additional trip for the selected resource 306.

In some embodiments, a portion of the timeline 334 is determined based on position tracking provided by a navigation system, such as the in-vehicle dispatch system 106 of FIG. 1A. For example, as described in relation to the emergency vehicle tracking engine 238 of FIG. 2, the GPS location of each emergency vehicle may be monitored. In this manner, the wheels rolling time, for example, may be determined based on a difference in GPS position exceeding a predetermined threshold, such as within 2 to 10 meters.

Further, in some embodiments, links to locations 336 of both a scene (e.g., location of the patient) 336a and a destination 336b are provided in the timeline 334. The links, when selected, for example, may zoom the map 302 into a region including a pick-up location 338a or a drop-off location 338b, respectively.

In some implementations, the window 318a includes a trip actions section 376a for adjusting trip information. The trip actions section 376a, for example, may be used to modify aspects of the trip, such as the contents of the timeline 334.

In some embodiments, the trip actions section 376a includes a time stamp control 378a configured, upon selection, to present the user with the ability to add one or more custom time stamps to the timeline 334. For example, a drop-down menu of time stamp options may be presented, upon selection of the time stamp control 378a, for user selection and data entry. Further to the example, the time stamp options may include events, actions, and/or activities such as, in some examples, vehicle check, inventory check, supervisor notified, page acknowledged, staging, and/or patient on board. In another example, a user may be provided the ability to enter any customized text as a time stamp label (e.g., text label). The user may be provided the option to add a time (e.g., clock time) to the time stamp and/or to drop onto a position within the timeline 334.

The trip actions section 376a, in some embodiments, includes an unassign control 380a configured, upon selection, to remove the assignment from the currently assigned transport vehicle (e.g., vehicle 110).

Turning to an example user interface display 340 of FIG. 3C, in some implementations, a dispatcher working at the user interface display 340 is presented with a call taking data entry pane 342 (e.g., data entry window, data entry tab, etc.) for intaking information regarding a medical dispatch request. The call taking data entry pane 342 includes a set of data entry sections 344, each section including one or more data entry fields. The data entry sections 344, for example, include a patient data entry section 344a, a caller data entry section 344b (which may be the patient or a separate individual), a pick-up location data entry section 344c, a resource assignment data entry section 344d, and a drop-off location data entry section 344e. A comments data entry section 344f provides the dispatcher with a text entry field to add other information regarding the call.

In the patient data entry section 344a, the dispatcher has the option to search for a patient by name. For example, as described in relation to FIG. 2, the clinical record access engine 224 may locate information regarding a known patient based upon entry of demographic information such as, in some examples, name, birth date, etc. (e.g., data other than name being available for entry upon selecting an advanced search option). The patient data entry section 344a presents patient Joe Smith, sex unknown, weight and birth date unknown.

In the caller data entry section 344b, a name, phone number, and call source (e.g., land line, mobile, internet protocol (IP) phone, etc.) are provided for data entry. In the event of a prescheduled trip, the caller data entry section 344b may be pre-populated with information (e.g., name, phone number, etc.) of the originator of the medical dispatch request.

Turning to the pick-up location data entry section 344c, an address of the patient is in the process of being entered (e.g., street address, city, state, zip code, etc.). Further, in some embodiments, the address data entry fields can accept a department, such as a department of a medical facility. Turning to the map 302, a location icon 345, in some embodiments, represents a point on the map 302 on which the dispatcher clicked as the location of the patient (e.g., a transport location icon placed at a map location corresponding to a geographic location of the patient transport site). For example, the map 302 may be an interactive graphical map (e.g., enabling user selection of a position in the interactive graphical map). As illustrated, a location copy menu 346 is open, providing options for the dispatcher to copy the selected location into data entry fields of the pick-up location data entry section 344c as a patient pickup location 348a or into data entry fields of the drop-off location data entry section 344e as a drop-off location 348b (e.g., a patient transport location or patient drop-off location, such as a medical facility, a name of a medical facility, a street address of a medical facility, etc.). The user may further edit, or perform a repositioning of, the pick-up location (or, alternatively, the drop-off location) entered, for example to specify a particular entry, apartment, department, unit number, etc.

As illustrated in the location copy menu 346, in some embodiments, GPS coordinates of the selected location may be copied to the call taking data entry pane 342 in addition to, or in lieu of, the identified street address.

In some embodiments, the location icon 345, after being placed on the interactive map 302, may be repositioned by a user (e.g., dragged) to an updated location, automatically causing an update in the corresponding data entry fields (e.g., of the pick-up location data entry section 344c or the drop-off location data entry section 344e).

As illustrated in a trip actions section 376b of the pop-up window 318b, in some implementations, a cancel trip control 384, upon selection, provides the user with the opportunity to cancel the trip.

In some implementations, an add trip control 382 of the trip actions section 376b provides the dispatcher with further trip configuration options. As illustrated in a pop-up menu 386, presented upon selection of the add trip control 382, the user may be provided the options to add a return trip via a return trip control 388 or add a leg via an add leg control 390. The return trip control 388 option, for example, may reverse the pickup location 348a and drop off location 348b for a second segment of the trip. Additionally, the add leg control 390 option may provide the user with the opportunity to add a stop before the pickup location 348a or between the pickup location 348a and the drop off location 348b. A stop prior to the pickup location 348a, for example, may enable the transport vehicle to obtain needed equipment.

In some implementations, the pop-up menu 386 generated through selection of the add trip control 382 includes a copy and modify trip option 392 for creating a new, similar trip to the one just produced. For example, in the circumstance of deploying multiple response vehicles for multiple patients injured on the scene of an accident, a portion of the information may be duplicated (e.g., pickup location 348a, drop off location 348b, comments 344f regarding the nature of the scene, etc.) to save the dispatcher time in providing assignment to the next vehicle.

Turning to an example user interface display 350 of FIG. 3D, in some implementations, the assignment data entry section 344d includes a drop-down menu 352 for selecting an available resource. As illustrated, a portion of the resources identified in the resources tab 306 are listed in the drop-down menu 352 by identifier and further by an estimated time en route (ETE). The estimated times, for example, may be calculated by the navigation services engine 236 of FIG. 2 and/or by the in-vehicle dispatch system 106. The resources in the drop-down menu 352 are arranged by fastest arrival time to slowest arrival time (e.g., 9:27 to 1:10:10). A dispatcher may cross-reference the fastest resource(s) presented in the drop-down menu 352 with the resource identifiers 310 in the resources tab 306, for example to ensure that the selected resource has an appropriate care level capability 312 (e.g., a vehicle service level) to meet the needs of the patient. The resources, in other examples, may be arranged based further on a nature of the medical emergency and/or a medical condition of the patient which may be indicative of vehicle capabilities required and/or a priority of response to the patient (e.g., critical, urgent, standard, etc.).

In some implementations, a current location of each in-the-field (e.g., en route, at destination, transporting, etc.) transport is identified on the map 302 by a transport position icon 354 (e.g., white circles bordered in either green or red). The current locations, for example, may be determined by the emergency vehicle tracking engine 238 of FIG. 2. Selection of one of the transport position icons 354, for example, may result in presentation of crew information and/or vehicle capabilities information. For example, selection of one of the transport position icons 354 may result in presentation of a pop-up window 318c, identifying the vehicle capability as ALS-GJ and the names of two crew members. Selection of a particular transport position icon 354, for example, may include a mouse click or a cursor hover.

Further, in some implementations, a set of post location icons 356 (e.g., depot location icons) represents locations of the various posts (depots) having available transports, and a set of vehicle location icons 358 (e.g., a set of individual EMS vehicle icons) represents locations of vehicles. In some embodiments, a first style, color, or type of icon may represent posts, and a second style, color, or type of icon may represent vehicles. The first icon and the second icon may be different in shape, color, or both. As illustrated, the post location icons are purple diamonds, and the vehicle location icons are purple circles. In some embodiments, certain post location icons 356 and/or vehicle location icons 358 are marked with a number. The number, for example, may indicate a number of locations of either posts or vehicles that are consolidated at a particular zoom level presented in the map 302.

In addition to zoom level adjustment, in some embodiments, the user may select a preferred map provider for overlapping the icons 356 and/or 358. For example, a user may be provided the opportunity to select from a number of different commercial mapping and/or navigational application such as, in illustration, Google® Maps by Google LLC of Mountain View, CA, Waze® by Waze Mobile of Mountain View, CA, MapQuest® by MapQuest Inc. of Denver, CO, and/or Apple Maps by Apple Inc. of Cupertino, CA.

In some implementations, selection of one of the post location icons 356 results in updating the call taking pane 342. For example, the assignment data entry section 344d may be filtered to only those resources located at the selected post 356. Selection of a particular post location icon 356, for example, may include a mouse click or a cursor hover.

Turning to FIG. 3E, in some implementations, as illustrated in an example user interface display 360, a dispatcher can review a listing of pending assignments 362 for matching with available resources of the resource list 306. The pending assignments, for example, may include scheduled and emergency dispatch assignments EMS crew and patient transport vehicles, as indicated by a priority 370 of the pending assignments list 362. The pending assignments, for example, may have been imported from a different call taking system, such as a PSAP system, a hospital or other medical facility, or another computer-generated request. The pending assignments list, in some embodiments, includes one or more transferred dispatch assignments of EMS crew and patient transport vehicles, as described in relation to FIG. 1C. Further, pending assignments list may include one or more prescheduled medical transport requests. As illustrated, each pending assignment includes a trip identifier 364, a nature of the request 366, a pick-up location 368, the priority 370, a type of care level 372, and a pick-up time 374. A dispatcher may match pending assignments with appropriate resources from the resource list 306. In some embodiments, the dispatcher may transfer one or more assignments to another agency, as described in relation to FIG. 1C.

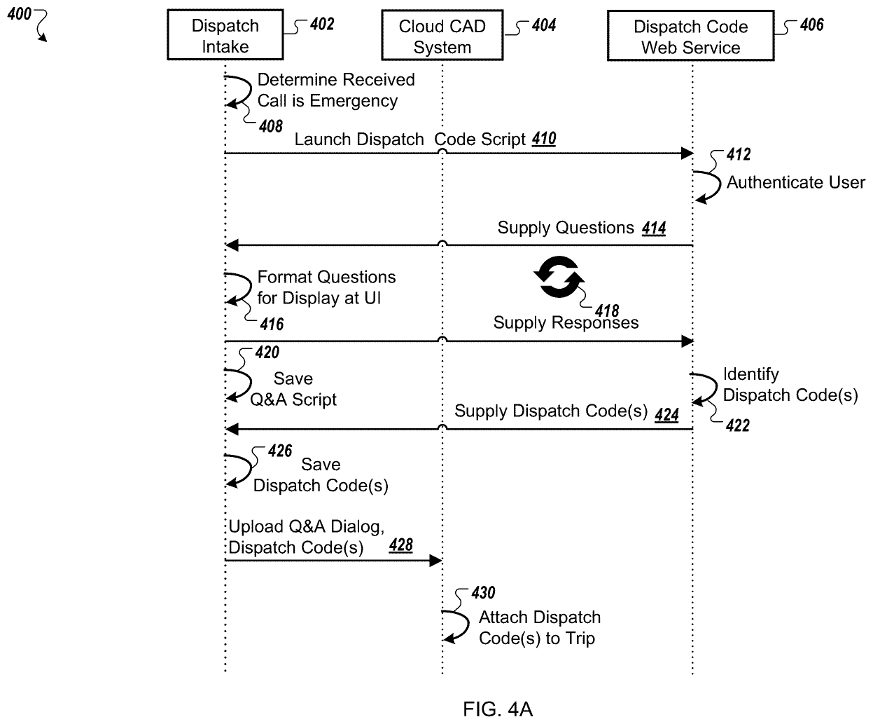

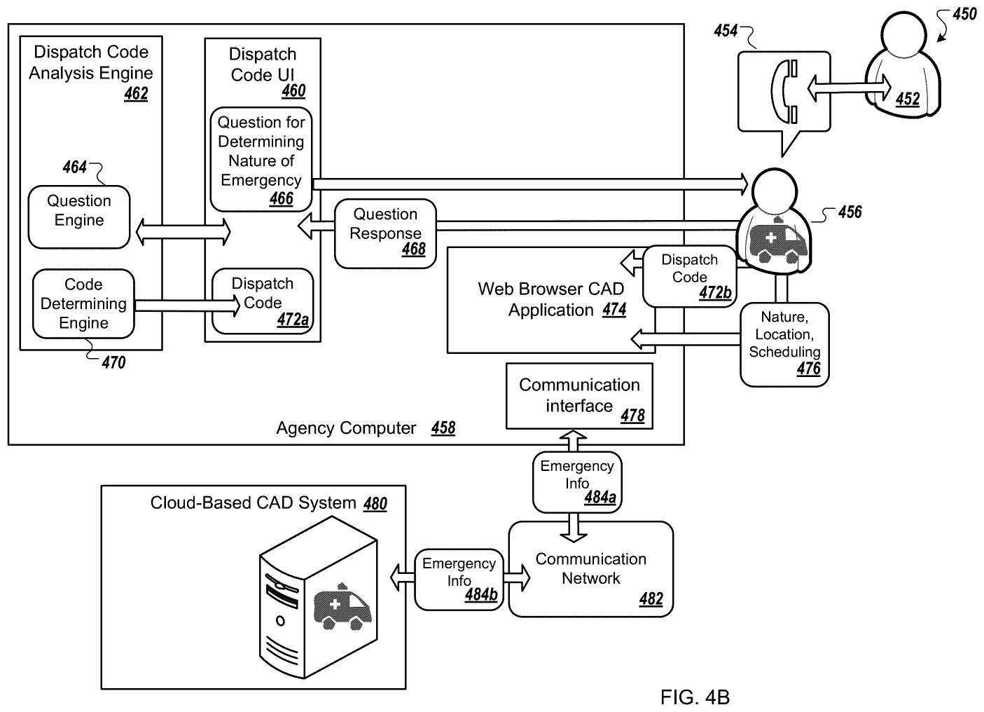

FIG. 4A is a swim lane diagram of an example process 400 for determining appropriate dispatch codes for dispatching EMS crews and patient transport vehicles. The process 400 represents example paths of communication between a dispatch intake system 402 (e.g., one of the agencies 204 of FIG. 2), a cloud-based CAD system 404 (e.g., the CAD system 202 of FIG. 2), and a dispatch code web service 406. Portions of the process 400 may be performed by the dispatch GUI engine 214, the dispatch engine 216, and/or the dispatch code engine 218 of FIG. 2.

In some implementations, the process 400 begins with determining, at the dispatch intake system 402, that a received call relates to a medical emergency (408). A dispatcher at an agency such as one of the agencies 204 of FIG. 2, for example, may answer a medical request call, such as a 911 call, and ask that the caller explain the circumstances of the medical emergency.

Upon determining that the call relates to a medical emergency, in some implementations, the dispatch intake system 402 launches (410) a dispatch code script with a dispatch code web service 406. The dispatch intake system 402 may connect, via an API, with the dispatch code web service 406, available to the dispatch intake system 402 via a network connection. The dispatch code web service 406, for example, may be provided by a third-party system. A dispatch user interface, such as the screen shot displays presented in FIG. 3A through FIG. 3E, may include a control (e.g., a triage dialogue control) for connecting with the dispatch code web service 406. The cloud-based CAD system 404, for example, may provide the graphical user interface including the control for launching the dispatch code script. In other embodiments, the dispatch code web service 406 may automatically launch, for example based on entry of information in the call taking pane 342 of FIG. 3C or based on natural language processing of a statement of a caller.

In some implementations, the dispatch code web service 406 authenticates (412) the user of the dispatch intake system 402 with a system providing the dispatch code web service 406. The authentication parameters, for example, may include a username, password, digital security key, and/or recognized network address (e.g., internet protocol (IP) address). The authentication information, for example, may be provided with the launch request (410).

In some implementations, the dispatch code web service 406 supplies (414) a set of questions to the dispatch intake system 402. The questions, for example, may include a series of questions to the caller designed to identify a nature of the medical emergency. The questions, in some examples, may be provided in a decision tree format, such that a path to a next question is selected based on the answer to a current question. The questions, for example, may be answered using binary (e.g., yes/no) and/or limited answer responses. The decision tree, for example, may be provided in a JavaScript Object Notation (JSON) format. In other embodiments, the decision tree is managed at the dispatch code web service 406, and the questions are supplied (414) one-by-one, based on responses (418) supplied by the dispatch intake system 402.

In some implementations, the dispatch intake system 402 formats (416) the questions for display at a user interface. Script data defining the inquiry decision tree, for example, may be formatted for presentation, one at a time, to the dispatcher to read to the caller. In other embodiments, the series of questions may be presented automatically through a text-to-voice application executed on the dispatch intake system 402. In further embodiments, the dispatch intake system 402 may forward the question decision tree to the cloud-based CAD system 404 for use in formatting the UI display.

In some implementations, the dispatch intake engine saves (420) the question and answer script for future use (e.g., with the next caller). In the event of receiving the entire decision tree, the download may be performed once (e.g., per session, per day, etc.) and used repeatedly with different callers.

In some implementations, responses given by the caller are supplied (418) to the dispatch code web service 406. As described above, the questions (414) and answers (418) may be provided back and forth between the dispatch intake system 402 and the dispatch code web service 406 until the dispatch code web service 406 identifies (422) one or more dispatch codes corresponding to the medical emergency.

In some implementations, the dispatch code web service 406 supplies one or more dispatch codes to the dispatch intake system 402 (424). The dispatch code(s), for example, may be numeric or alphanumeric. Further, each dispatch code may include a brief text description.

In some implementations, the dispatch code(s) are saved (426) by the dispatch intake system 402. The dispatch intake system 402, for example, may associate the dispatch code(s) with other data related to the call.

In some implementations, the dispatch intake system 402 uploads the question and answer (Q&A) dialog (e.g., path taken with the caller, the entire decision tree and answers provided by the caller, etc.) along with the dispatch code(s) 428 to the cloud-based CAD system 404. The user interface, for example, may access the dispatch code(s) and the Q&A dialog from a storage region used by the dispatch intake system 402. In another example, the dispatcher may enter the dispatch code(s) into the user interface provided by the cloud CAD system 404, such as the call taking pane 342 of FIG. 3C.

In some implementations, the cloud-based CAD system attaches the dispatch code(s) to the trip (430). For example, the cloud-based CAD system 404 may store the dispatch codes(s) in association with other trip information entered into the graphical user interface. In reference to FIG. 2, the dispatch code engine 218 may store the dispatch codes 260 in correspondence with the patient data 256, patient location data 258, and/or other trip information.

Although illustrated as a particular series of operations, in some embodiments, the process 400 includes more or fewer operations. For example, in some embodiments, the call is assumed to be a medical emergency and the dispatch code script is launched (410) automatically without first determining (408) whether the call regards an emergency. In some embodiments, certain operations of the process 400 may be performed in a different order and/or concurrently. For example, in some embodiments, while the dispatch intake system 402 and the dispatch code web service 406 are trading questions (414) and responses (418), the dispatch code web service 406 may be repeatedly attempting to identify the appropriate dispatch code(s) 422. Other modifications to the process 400 are possible while remaining in the spirit and scope of the process 400.

FIG. 4B is a block diagram of an example system 450 for determining appropriate dispatch codes for dispatching EMS crews and patient transport vehicles. The system 450, for example, may be used to perform operations similar to those described in relation to the process 400 of FIG. 4A, although the architecture of the system 450 differs from the distribution of the systems 402, 404, and 406 of FIG. 4A. The system 450 includes an agency computer 458, such as one of the agencies 204 of FIG. 2, executing a web browser CAD application 474 driven by a cloud-based CAD system 480 via a communication network 482. The agency computer 458 may be executing a dispatch code analysis engine 462 for guiding a question and answer routine between a dispatcher 456 and a caller 452 through prompts presented via a dispatch code user interface (UI) 460 of the agency computer 458.

In some implementations, a caller 452 is connected via a voice connection 454 with a dispatcher 456 working at the agency computer 458. A question engine 464 of the dispatch code analysis engine 462 supplies a question 466 for determining the nature of the medical emergency for presentation by the dispatch code UI 460 at the agency computer 458.

In some implementations, a question response 468 is entered by the dispatcher 456 at the agency computer 458 via the dispatch code UI 460. The question response 468 is provided to the dispatch code analysis engine 462 to determine a next question by the question engine 464 or to determine and provide a corresponding dispatch code 472a using a code determining engine 470. The supplying of questions 466 by the dispatcher 456 and receiving question responses 468 from the caller 452 may continue until the code determining engine 470 identifies the dispatch code 472a as being a match to the information supplied by the caller 452 via the question responses 468.

In some implementations, the dispatch code 472a is presented to the dispatcher 456 at the dispatch code user interface 460. The dispatch code 472 may be copied as dispatch code 472b, for example by the dispatcher 456, into a web browser CAD application 474. Further, the dispatcher 456 may enter additional information 476 regarding the medical emergency request such as a nature of the request, a location of the patient, and scheduling of services, such as medical transport.

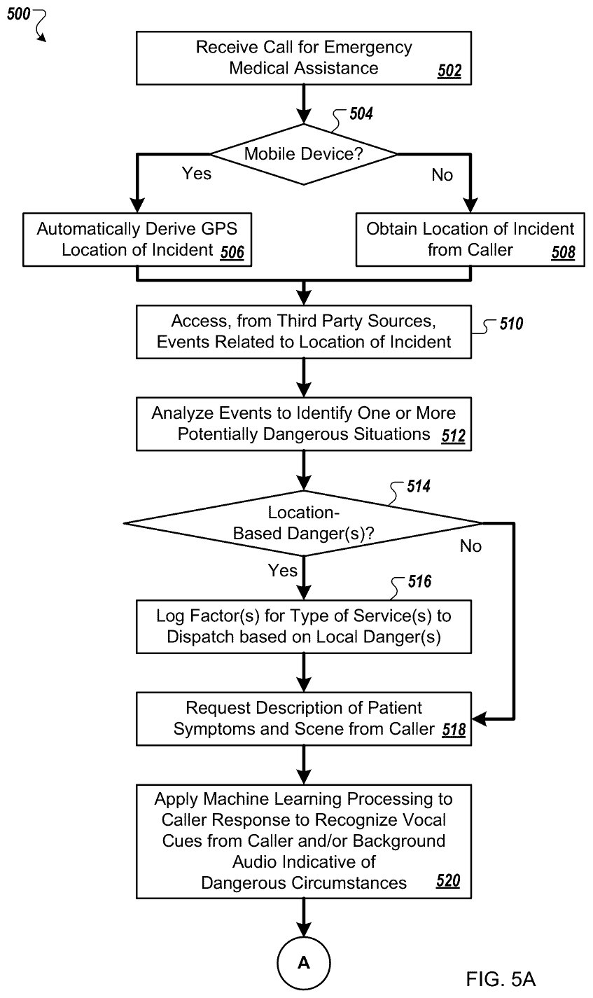

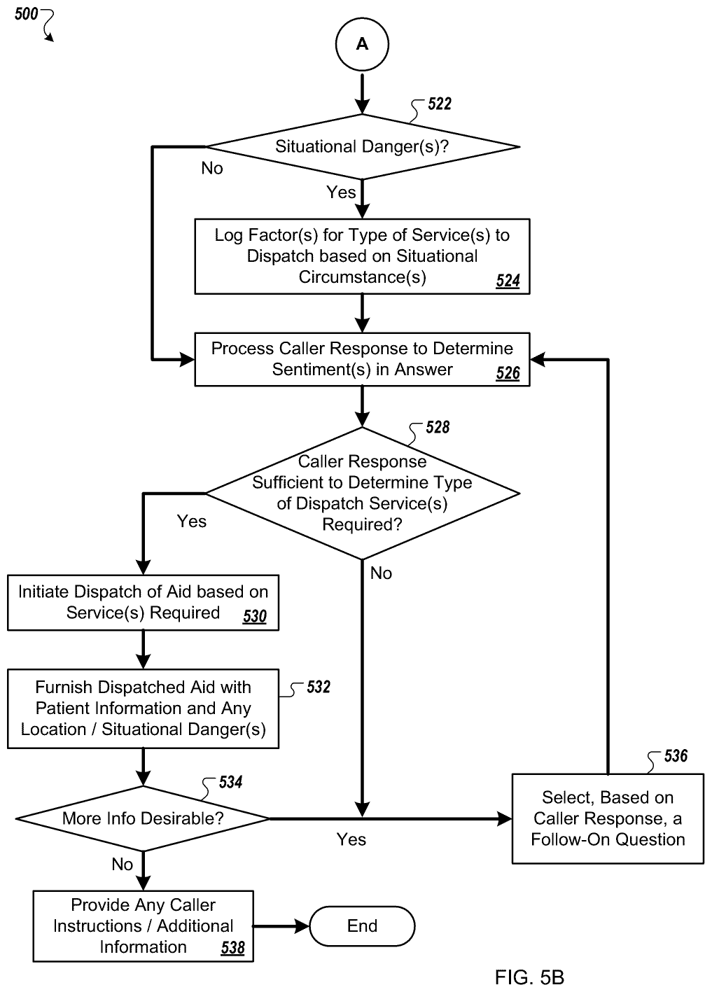

In some implementations, the dispatch code 472b and the additional information 476 are provided, over the communication network 482 via the communication interface 478, to the cloud-based CAD system 480 as emergency information 484a. The CAD system, in response, may supplement the emergency information as emergency information 484b, transferred back to the web browser CAD application 474. The supplemented emergency information 484b, in some examples, may include transport location and availability information (e.g., as described in relation to the equipping/staffing decision engine 226, the transport scheduling engine 228, the vehicle equipment inventory engine 232, and/or the emergency vehicle tracking engine 238 of FIG. 2), refined route navigation information (e.g., as described in relation to the navigation services engine 236 of FIG. 2), supplemental patient information (e.g., as described in relation to the clinical record access engine 224 of FIG. 2), and/or additional information regarding the location (e.g., as described in relation to a method 500 of FIG. 5A and FIG. 5B).

FIG. 5A and FIG. 5B illustrate a flow chart of the example method 500 for determining appropriate dispatch services based on context and cues associated with a request for medical services. The method 500, for example, may be performed by the CAD system 202 of FIG. 2 in coordination with one of the agencies 204. Portions of the method 500, in some examples, may be performed by the dispatch GUI engine 214, the dispatch engine 216, the dispatch code engine 218, and/or the natural language processing engine 220 of FIG. 2.

In some implementations, the method 500 begins with receiving a call for emergency medical assistance (502). As described above in relation to FIG. 1A through FIG. 1C, the call may be received at an EMS agency, CAD system, PSAP, or other emergency response organization.

In some implementations, if the caller is using a mobile device or other positioning system (e.g., GPS) enabled device (504), the location of the incident is automatically derived (506). The navigation services engine 236 of FIG. 2, for example, may derive the GPS data from a caller identification or other data shared by the GPS-enabled device.

In some implementations, if the call is not from a GPS-enabled device or positioning data is otherwise not available (504), the location of the incident is obtained from the caller (508). For example, a dispatcher may enter the location at a graphical user interface, such as the pick-up location data entry section 344c of the user interface display 340 of FIG. 3C.

In some implementations, events related to the location of the incident are accessed from one or more third party sources (510). For example, traffic information may be gathered from a navigation system such as the navigation services engine 236 of FIG. 2. Weather data may be gathered, in another example, from one or more weather sources. Police activity data, in another example, may be gathered from a social media feed and/or short band radio feed of a local police unit. In a further example, news data and/or local government data may be gathered from one or more online resources.

In some embodiments, a vicinity surrounding the location is used for identifying events. The vicinity reviewed, in some examples, may include a street, a multi-street region (e.g., 4-block area, 8-block area), and/or a geographically bounded region (e.g., an area expanding from the location with a set radius such as a quarter mile or half mile radius). The size of the vicinity, for example, may depend upon the news source (e.g., weather data may be reviewed in relation to a wider vicinity than news or government data).

In some implementations, the events are analyzed to identify one or more potentially danger situations (512). Weather data may identify a catastrophic event such as, in some examples, a flood, wildfire, hurricane, tornado touchdown, or lightning strike. Police activity data, in another example, may identify an ongoing criminal incident such as, in some examples, a riot, terrorist attack, street fight, or manhunt. In a further example, news data and/or local government data may identify an event within the vicinity such as a parade, sports event, festival, political rally, or public demonstration which could lead to difficult or dangerous circumstances for accessing the victim of a medical emergency.

In some implementations, if one or more location-based dangers are identified (514), one or more factors for types of services to dispatch based on the location-based dangers are logged (516). The factors, for example, can include a type of vehicle to deploy (e.g., a vehicle with chains on the tires in blizzard conditions, a vehicle with minimal footprint in crowded conditions, etc.). Further, the factors can include a number of personnel to deploy (e.g., an extra crew member for crowd control). In some embodiments, the factors include deploying other services, such as requesting community-based services or police services to assist and/or protect the crew in dealing with the potentially dangerous event. For example, extra teams may be deployed based on additionally determined patients at the scene. Further, a mass casualty situation, such as a shooting or a large scale accident like a train derailment, may cause deployment of most or all available resources depending on the size of a particular agency. In some instances, crews may be dispatched from multiple agencies. Logging the factor(s), for example, may involve adding one or more location-based danger codes to information associated with the emergency medical services request. The codes, in some cases, are associated with brief descriptions, such as, in some examples, “fire on premise” or “icy road conditions in area.” The codes and/or brief descriptions, in some embodiments, may be automatically added to a graphical user interface of the dispatcher, for example in the comments data entry section 344f of the user interface display 340 of FIG. 3C.

In some implementations, a description of patient symptoms and the scene (e.g., position of the patient, surroundings, etc.) are requested of the caller (518). A dispatcher, for example, may ask the caller to briefly describe the medical emergency (e.g., what happened, what is the state of the patient, etc.).

In some implementations, machine learning processing is applied to the caller response to recognize vocal cues from the caller and/or background audio indicative of dangerous circumstances (520). The vocal cues, in some examples, may include screaming, shrieking, crying, stuttering, fearful whispering, high pitched and/or rapid speech, and/or repetitive non-verbal vocalizations. One or more machine learning classifiers used to analyze the caller response for vocal cues may be trained, in some examples, to identify indications of fear, terror, threat, shock, or coercion. The background audio cues, in some examples, may include sirens, building alarms, car alarms, yelling, screaming, threatening-toned utterances, gun shots, slapping/striking of skin, growling, and/or banging. In illustrations, the background audio cues may include a threatening individual at the scene. One or more machine learning classifiers used to analyze the background audio may be trained to identify background audio cues associated with, in some examples, domestic violence, assault, rioting, and/or criminal threat (breaking and entering, hostage situation, etc.).

Turning to FIG. 5B, in some implementations, if one or more situational dangers are detected (522), one or more factors related to the types of services to dispatch based on situational circumstances are logged (524). The factors, in some examples, can include deploying other services, such as requesting mental health services if an emotionally distressed individual is on premise (including the caller) or police services to assist and/or protect the crew in dealing with the potentially dangerous event. The extent and variety of deployed services may vary based on specifics of the event. Events that involve hazardous materials or contagious diseases may require a wider range of services and providers than a cardiac event for a single victim. For example, a chemical fire may require special equipment or procedures, while an emergency in a remote area may require flight operations.

In some implementations, the caller response is processed to determine one or more sentiments in the caller's answer (526). The terms used by the caller, for example, may be indicative of dangerous circumstances and/or a situation that would benefit from additional services. The natural language processing engine 220 of FIG. 2, for example, may analyze the audio of the caller to identify the terminology used. One or more machine learning classifiers trained to derive sentiment from language cues may analyze the output of the natural language processing to determine a caller sentiment. The sentiment may be indicative of an emotion of the caller, a mental health factor of the caller, and/or a comprehension level of the caller to an instruction or a question posed to the caller. Sentiment, in some examples, may relate to urgency, fear, confusion, and/or rage. The sentiment, for example, may be used to adjust previously identified factors and/or add factors. Each factor, for example, may be associated by a confidence level or rating applied by the machine learning analyses above. In analyzing the individual components (e.g., vocal cues, background cues, and/or sentiment cues), the confidence associated with each factor may be refined.

If the caller response is sufficient to determine the type of dispatch services required (528), in some implementations, dispatch of aid is initiated based on services required (530). For example, a patient found unconscious floating face down in a pool may require no further medical questions to determine that a swift, particular response is required. The services determined to be required, in some embodiments, may be derived in part based on a threshold confidence level associated with the machine learning analyses above.

In some implementations, the dispatched aid is furnished with patient information and any location and/or situational dangers identified (532). For example, the information may be provided from the cloud-based SaaS platform 102 to the emergency vehicle 124 as described in relation to FIG. 1A. The patient information, for example, may include information similar to that illustrated as patient information 332 of FIG. 3B.

In some implementations, if the caller response is insufficient (528) or if more information from the caller is desired (534), a follow-on question is selected based on caller response (536). The follow-on question, for example, may be selected by the dispatch code engine 218 using the caller intake script 262 of FIG. 2. For example, follow-on questions may be determined as described in relation to the process 400 of FIG. 4A and/or the system 450 of FIG. 4B.

In some implementations, the caller responses are processed for sentiment (526) and additional questions are provided (536) until no further information is desired (536). For example, as described in relation to the dispatch code engine 218, the process 400 of FIG. 4A and the system 450 of FIG. 4B, a question and answer script may be followed (e.g., a decision tree of questions and possible responses traversed) until one or more dispatch codes are determined).

In some implementations, the caller is provided with instructions and/or additional information (538). The further information and/or instructions, for example, may be provided as described in relation to the caller instruction engine 240 of FIG. 2.

Although illustrated as a particular series of operations, in some embodiments, the method 500 includes more or fewer operations. For example, other medical requests for assistance within a same vicinity may be analyzed for evidence of a widespread dangerous situation (e.g., natural or manmade disaster causing multiple injuries). In examples where the caller is the patient, the caller response may be processed to analyze the prosody of the answer. The prosody, for example, may be indicative of one or more medical symptom factors such as stroke (slurring), shock, and/or a mental health condition. The nature of the emergency (e.g., dispatch codes) may be adjusted based on the prosody providing evidence of one or more medical conditions. In some embodiments, certain operations of the method 500 may be performed in a different order and/or concurrently. For example, rather than processing subsequent caller responses only for sentiment (526), the vocal cues and background audio may be analyzed in relation to each answer (520). Further, the vocal cues, background audio (520) and sentiment (526) may be analyzed concurrently. Other modifications to the method 500 are possible while remaining in the spirit and scope of the method 500.

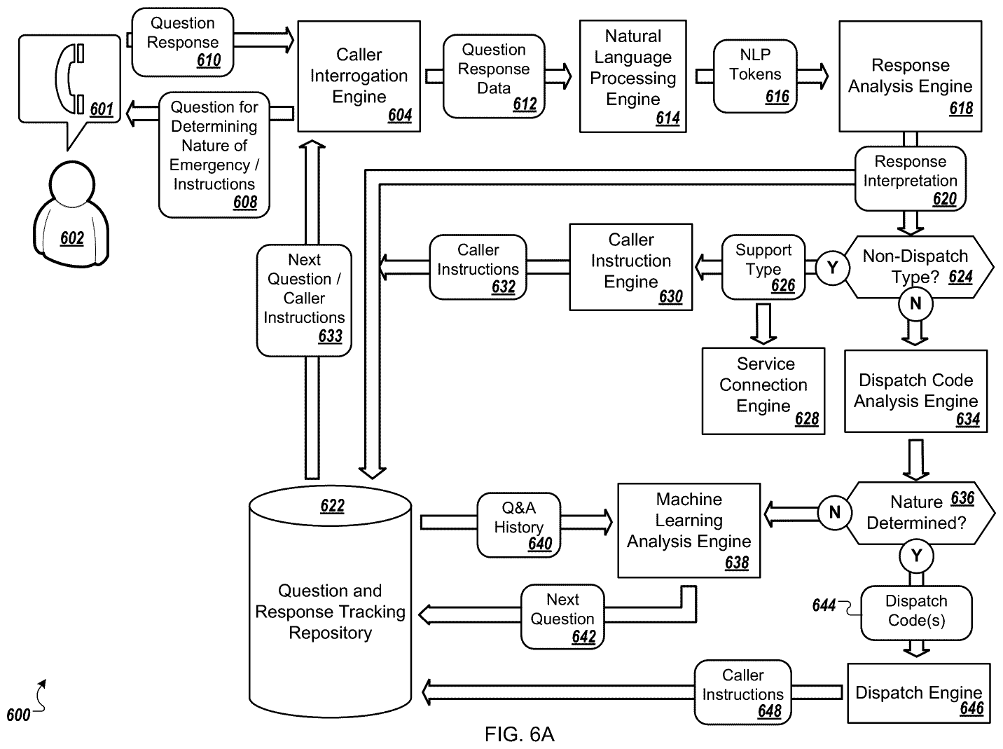

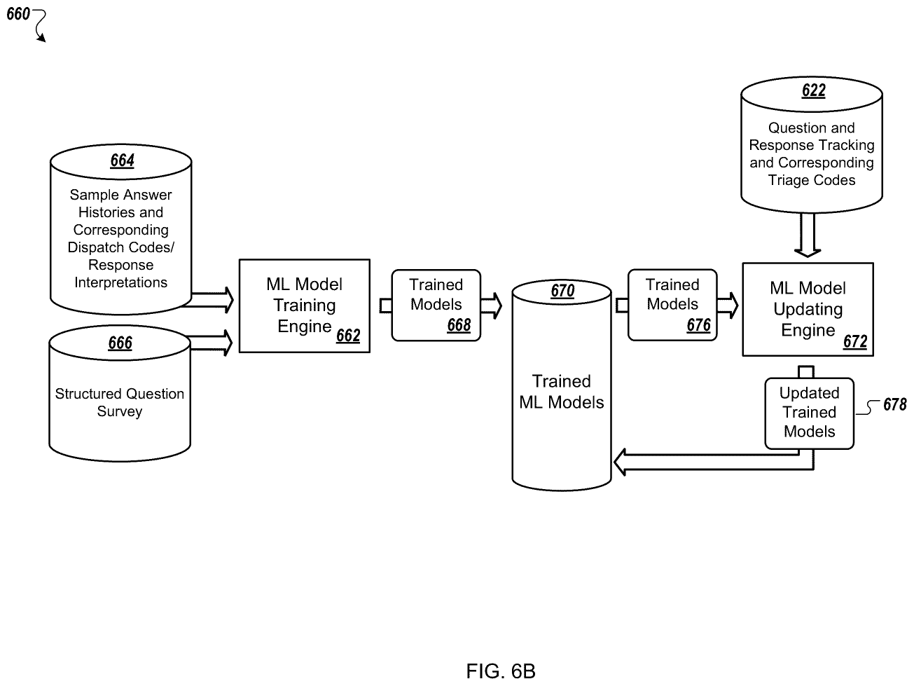

FIG. 6A is a flow diagram of an example process 600 for applying natural language processing to determining dispatch codes for dispatching EMS crew and patient transport vehicles. The process 600, for example, may be performed by the CAD system 202 of FIG. 2. Portions of the process 600, for example, may be performed by the dispatch engine 216, the dispatch code engine 218, the natural language processing engine 220, the caller instruction engine 240, and/or the service connection engine 246 of FIG. 2.

In some implementations, the process 600 begins with receiving an emergency medical request call 601 from a caller 602. The caller may be a patient, a caregiver, or another witness to a medical emergency. The call 601, in some examples, may be connected via a telephone, smart phone, voice call or other voice-enabled application executing on a computing device, and/or a radio wave communication device (e.g., walkie-talkie). The computing device executing the voice-enabled application, in some examples, may be a wearable computing device (e.g., emergency request device worn on a lanyard by an elderly person, smart watch, smart glasses, etc.), a wearable medical device (e.g., a Holter monitor, wearable cardiac monitor/defibrillator, etc.), a tablet computing device, and/or a personal computer (e.g., laptop computer, desktop computer, etc.). Further, the computing device executing the voice-enabled application may be the computing system of a vehicle, such as a smart car.

In some implementations, a caller interrogation engine 604 supplies a question 608 for determining the nature of the medical emergency. The question 608, for example, may be an initial inquiry requesting a description of the medical emergency. The question may be provided, in some examples, via a live dispatcher, a text-to-voice application, and/or a pre-recorded message.

The initial inquiry, in some embodiments, includes obtaining a language of the caller 602 and/or demographic information of the caller 602 to assist in analysis of responses provided by the caller 602. For example, the demographic information may include a sex, age, location of residence, and/or primary language spoken.

In some implementations, a question response 610 is uttered by the caller 602. The caller interrogation engine 604 may provide a digital audio recording or stream of the question response 610 as question response data 612 to a natural language processing engine 614.

In some implementations, the natural language processing engine 614 analyzes the question response data 612 and breaks down the utterances into a set of NLP tokens 616. The NLP tokens 616, for example, may capture symptom terms, injury terms, disease terms, and/or disorder terms spoken by the caller 602. Further, the NLP tokens 616 may include affirmative/negating terms (e.g., yes/know).

The NLP engine 614, in some embodiments, analyzes the question response data 612 in view of the language of the caller and/or demographic information of the caller. For example, sex and/or age of the caller 602 may be used to refine the natural language processing to one trained to recognize words uttered by persons of a particular sex or of a particular age range (e.g., a child caller may be understood differently than an elderly caller). Further, a location of residence and/or primary language spoken may provide clues to an accent of the caller 602, thereby informing the NLP engine 614 to attempt analyzing in view of a particular accent or speech pattern common to a country or region.

In some implementations, the NLP tokens 616 are provided to a response analysis engine 618 to interpret the response to the question 608. The response analysis engine 618, for example, analyze the NLP tokens in view of synonymous and/or similar terms to match the words and/or phrases spoken by the caller 602 with anticipated terminology (e.g., being responsive to the particular question 608 posed. The response analysis engine 618, for example, may be trained to interpret responses based at least in part on question content and/or anticipated response (e.g., yes/no, yes/no/unsure, or other limited answer set). The response analysis engine 618 may produce a response interpretation 620. The response interpretation 620, for example, may be one of a set of potential responses to the question 608, based on the content/sentiment (e.g., positive or negative language) of the NLP tokens 616 of the question response 610.

In some implementations, the response interpretation 620 is stored in a question and response tracking repository 622. The question and response tracking repository 622, for example, may be used to build a history of posed questions 608 and response interpretations 620.

In some implementations, the response interpretation 620 is analyzed to determine if it is indicative of a non-dispatch type of event (624). Some callers may call about a non-emergency situation and/or non-EMS type medical emergency (e.g., suicidal caller, mental health issue, etc.). The response analysis engine 618, for example, may determine whether the response interpretation 620 is indicative of a non-dispatch type issue.

If the nature of the call 601 does not match with medical dispatch, in some implementations, a support type 626 is determined (626). The support type, for example, may be determined by the response analysis engine 618 (e.g., similar to the capabilities of the equipping/staffing decision engine 226 and/or the resource allocation engine 234 of FIG. 2). The support, in some examples, can include a telehealth provider, community intervention services, community-based outreach services, mental health services, and/or social services.

In some implementations, a service connection engine 628 manages allocation of the support type 626. The service connection engine 628, for example, may perform the operations of the service connection engine 246 of FIG. 2.

In some implementations, a caller instruction engine 630 determines caller instructions 632 to provide to the caller 602 related to the support type 626. The caller instructions 632, in some examples, may include identification of one or more services dispatched by the service connection engine 628, contact information for the caller 602 to reach out to an identified service, and/or instructions of what to do prior to arrival of the services. The caller instruction engine 630, for example, may perform some of the operations described in relation to the caller instruction engine 240 of FIG. 2.

In some implementations, the caller instructions 632 are stored to the question and response tracking repository 622, where they may be obtained by the caller interrogation engine 604 as caller instructions 633 and read/spoken to the caller 602 as instructions 608.

In some implementations, if the response interpretation 620 does correspond to a dispatch type 624, a dispatch code analysis engine 634 analyzes the response interpretation 620 to identify whether a nature of the medical emergency can be determined 636 based upon the information provided so far by the caller 602. The dispatch code analysis engine 634, for example, may analyze the response interpretation 620 in view of the question 608 as well as in view of previous questions and responses stored in the question and response tracking repository 622. The dispatch code analysis engine 634, for example, may perform the operations described in relation to the dispatch code engine 218 of FIG. 2, the dispatch code web service 406 of FIG. 4A, and/or the code determining engine 470 of FIG. 4B.

In some implementations, if the dispatch code analysis engine 634 is incapable of determining the nature of the medical emergency based upon the present information (636), a machine learning analysis engine 638 analyzes the question and answer history 640 between the caller 602 and the caller interrogation engine 604 to determine a next question 642. The machine learning analysis engine 638, for example, may be trained with historic question and answer script patterns and outcomes to determine a shortest path to obtaining the necessary information from the caller 602 for determining the nature of the medical emergency (e.g., corresponding dispatch codes 644). The machine learning analysis engine 638, for example, may be trained using the Q&A script saved (420) by the process 400 of FIG. 4A.

In some embodiments, the machine learning analysis engine 638 selects the next question 642 from a set of interrogation records, each record including a limited answer question and a set of potential answers corresponding to the limited answer question. The limited answer questions, for example, may follow one or more protocols of the International Academies of Emergency Dispatch (IAED). Further, each interrogation record may include a medical condition indicator corresponding to at least one answer of the set of potential answers.

Turning to FIG. 6B, a flow diagram of an example process 660 for training machine learning algorithms to analyze caller responses and anticipate a shortest path of questioning to determine dispatch codes for dispatching EMS crews and patient transport vehicles is illustrated. The process 660, for example, may be used to train the machine learning analysis engine 638 of FIG. 6A. The process 660 includes a machine learning model training engine 662 for training one or more trained models 668 using sample answer histories and corresponding dispatch codes/response interpretations 664. The machine learning model training engine 662, for example, may use a gradient boosting machine (GBM), which is a tree-based model that is useful in regression and classification. Other types of machine learning algorithms useful in finding a shortest path of questioning to determining the nature of the medical emergency may include logistic regression, K-nearest neighbors (KNN), one class support vector machine (SVM), or other tree-based models such as a decision tree or random forest (RF). In an example, large language neural network models (LLMs) may be employed to interpret the language and context of a conversation and infer dispatch acuity with domain training specific to dispatch.

In some implementations, the machine learning model training engine 662 is further provided with at least one structured question survey 666. For example, as discussed in relation to the set of questions (414) of FIG. 4A, the structured question survey 666 may include a decision tree of questions and dispatch code(s) resulting from each potential path through the decision tree. The structured question survey(s) 666, for example, may be used in regression-based training to analyze for efficient pathways to resultant dispatch codes.

In some implementations, the trained models 668 are stored to a trained machine learning model data store 670, for example to be used by the machine learning analysis engine 638 of FIG. 6A.

Returning to FIG. 6A, in some implementations, the next question 642 determined by the machine learning analysis engine 638 is provided, via the question and response tracking repository 622, to the caller interrogation engine 604 as the next question 633. The caller interrogation engine 604, for example, may present the next question 633 as the question 608 at a dispatch user interface for a dispatcher to read and/or cause a recording of the question 608 to be automatically played/read to the caller 602.

In some implementations, the process 600 continues to supply next questions 633 and receive question responses 610 until the nature of the medical emergency is determined (636) by the dispatch code analysis engine 634. The dispatch code analysis engine 634, in some embodiments, analyzes medical condition indicators corresponding to one or more answers provided by the caller 602 to identify the corresponding dispatch code(s). The dispatch code analysis engine 634, for example, may apply a set of interrogation rules to matching dispatch codes with medical condition indicators of the interrogation records discussed above. The dispatch code analysis engine 634, for example, may provide one or more dispatch codes 644 that are responsive to the medical emergency described by the caller 602 during the question and answer session. The dispatch codes 644 may be provided to a dispatch engine 646 (e.g., the dispatch engine 216 of FIG. 2) for launching a dispatch process for scheduling and dispatching services such as emergency medical transport to a patient.

In some implementations, the dispatch engine 646 determines caller instructions 648 to provide to the caller 602. The caller instructions 648, for example, may be stored to the question and response tracking repository 622 for access by the caller interrogation engine 604 which manages presentation to a dispatcher/reading to the caller 602. The caller instructions 648, for example, may be determined by the caller instruction engine 240 of FIG. 2.

Turning to FIG. 6B, in some implementations, results of the process 600, as captured in the question and response tracking repository 622, may be used by a machine learning model updating engine 672 to update one or more of the stored trained models 676. The results, for example, include identifications of the limited answer questions, the corresponding answer of the set of potential answers, the answer provide by the caller (if a synonym to one of the limited answer questions converted by the natural language processing engine 614 and/or the response analysis engine 618), and the medical condition indicator identified, if any, based on the corresponding answer. The updated trained models 678 developed through ongoing learning provided by the logged question and answer sessions, for example, may be fed back to the trained machine learning models data store 670 for application in future question and answer sessions.

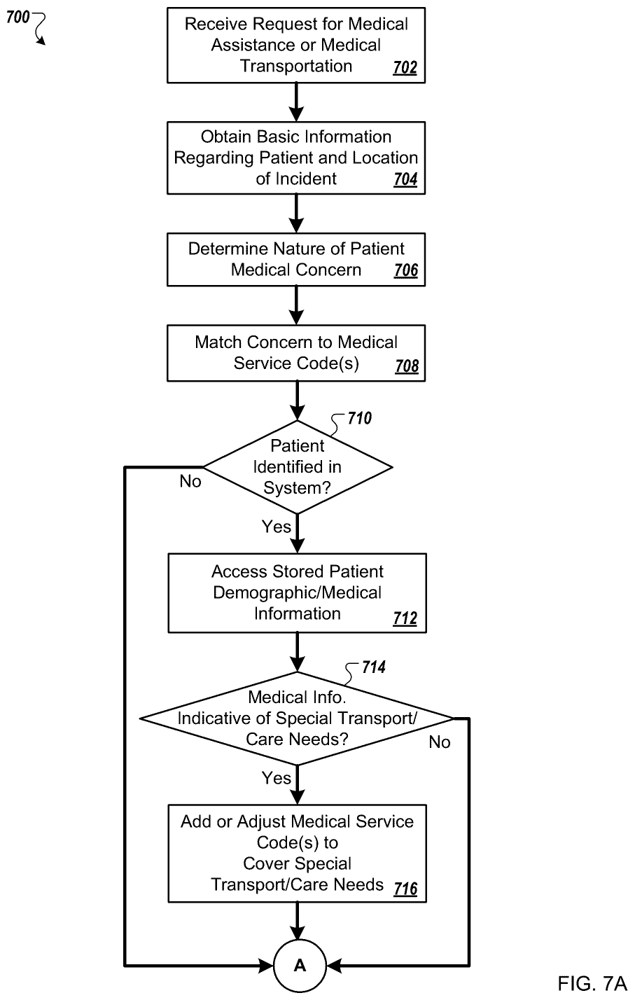

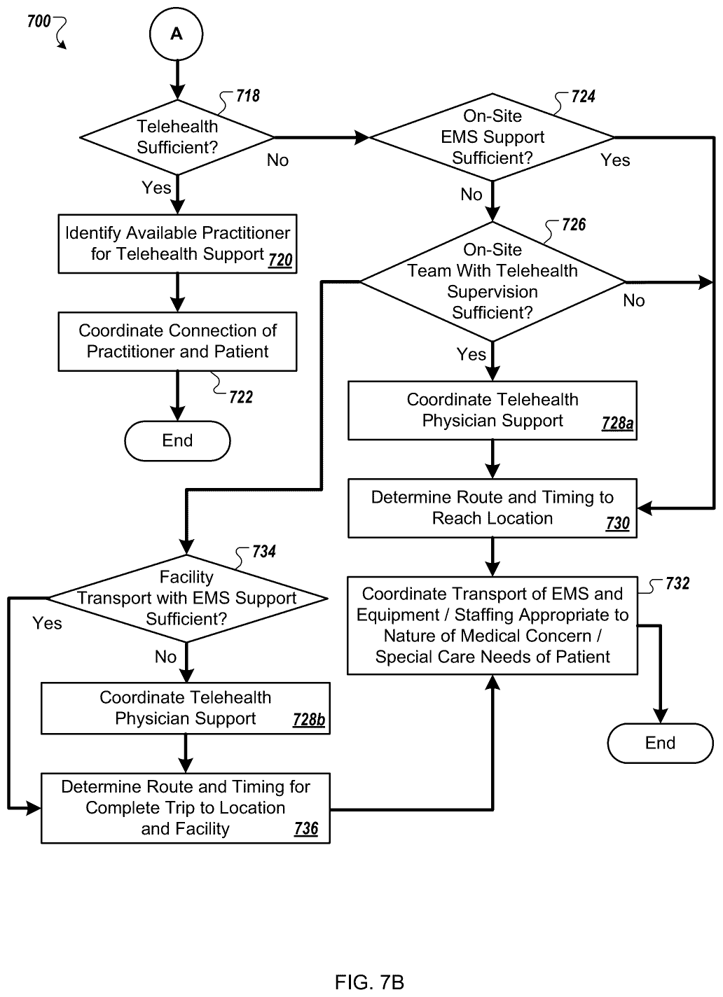

FIG. 7A and FIG. 7B illustrate a flow chart of an example method 700 for determining and coordinating appropriate staffing to respond to a medical assistance call. The method 700, for example, may be performed by the CAD system 202 of FIG. 2 in cooperation with one of the agencies 204, telehealth practitioners 208, and/or transport vehicles 210. Portions of the method 700 may be performed, in some examples, by the dispatch GUI engine 214, the dispatch engine 216, the dispatch code engine 218, the EMS request intake engine 222, the clinical record access engine 224, the equipping/staffing decision engine 226, the transport scheduling engine 228, the vehicle capabilities decision engine 230, the vehicle equipment inventory engine 232, the resource allocation engine 234, the navigation services engine 236, the service connection engine 246, the triage professional scheduling engine 248, and/or the triage communication support engine 250 of FIG. 2.

In some implementations, the method 700 begins with receiving a request for medical assistance or medical transportation (702). The request may be received, in some examples, via call, facsimile, and/or computer transmission, as described above.

In some implementations, basic information regarding a patient and a location of the medical incident are obtained (704). The information may be obtained, for example, by a dispatcher at one of the agencies 204 interacting with the dispatch engine 216 via the dispatch GUI engine 214, as described in relation to FIG. 2. For example, the information may be captured via the graphical user interface display 340 of FIG. 3C.

In some implementations, the nature of the patient medical concern is determined (706). In some examples, the patient medical concern may be identified via the dispatch code engine 218 of FIG. 2, the process 400 of FIG. 4A, the dispatch code analysis engine 462 of FIG. 4B, and/or the process 600 of FIG. 6A.

In some implementations, the nature of the patient's medical concern is matched to one or more medical service codes (708). The medical service codes, for example, may be triage codes, dispatch codes, determinant codes, and/or insurance billing codes. For example, the medical service codes may be selected from the dispatch codes 260 of FIG. 2.

In some implementations, if the patient is identified in the system (710), stored patient demographic information and/or medical information is accessed (712). The information may be accessed, for example, using the clinical record access engine 224 of FIG. 2 by cross-referencing clinical records with a portion of the initial patient information.

In some implementations, if the patient's medical information is indicative of special transport needs and/or special care needs (714), a medical service code is added and/or one or more of the medical service codes is adjusted to cover the special transport needs and/or special care needs (716). In some illustrative examples, the special needs may relate to the patient being wheelchair bound, requiring an oxygen tank, and/or needing oversized gurney/equipment due to body size. Other circumstances that may require specialty care can include, in some examples, neonatal care, hazardous materials emergencies, organ transfer, and/or water emergencies requiring boats as the emergency rescue vehicles. Care may be specialized due to the type of transport vehicle for victims and/or crew, the rescue equipment needed, and/or the training required for a crew in order to effectively respond.

Turning to FIG. 7B, in some implementations, if it is determined that telehealth is sufficient as a response service (718), an available practitioner is identified for telehealth support (720). For example, the medical service code(s) may be reviewed to determine a level of care and/or appropriate service(s) to respond to the patient's needs. The equipping/staffing decision engine 226 of FIG. 2, for example, may determine whether telehealth is a sufficient response. Telehealth support, in some examples, may include a nurse hotline, a mental health professional, a suicide prevention volunteer or professional, and or a community outreach volunteer or professional.

In some implementations, a connection between the identified telehealth practitioner and patient is coordinated (722). The connection, for example, may be managed as described in relation to the service connection engine 246 of FIG. 2.

In some implementations, if telehealth response is not deemed sufficient for the patient's medical needs (718), it is determined if on-site EMS support is sufficient (724). Rather than transporting a patient to the hospital, in some circumstances, emergency medical technician (EMT) or other professional triage support may be performed on-premises for a patient.

If on-site EMS support is deemed sufficient (724), in some implementations, a route and timing is determined to reach the location of the patient (730). For example, the navigation services engine 236 of FIG. 2 may determine the route and timing.

After determining route and timing, in some implementations, transport of EMS and equipment and/or staffing appropriate to the nature of the medical concern and, where designated, the special care needs of the patient, is coordinated (732). For example, the transport scheduling engine 228, the vehicle capabilities decision engine 230, the vehicle equipment inventory engine 232, and/or the resource allocation engine 234 may be applied to coordinating transport. A dispatcher working with the user interface display 350 of FIG. 3D and/or the user interface display 360 of FIG. 3E, for example, may assign appropriate resources for dispatch.

In some implementations, if on-site EMS support was not deemed sufficient (724), it is determined whether an on-site team with telehealth supervision would be sufficient (726). For example, to provide certain medical interventions, the transport crew may require physician supervision in performing medical treatments.

If an on-site EMS team with telehealth supervision is deemed sufficient (726), in some implementations, telehealth physician support is coordinated (728). For example, the triage professional scheduling engine 248 of FIG. 2 may schedule support of one of the telehealth practitioners 208, as described in relation to FIG. 2. Further, the triage communication support engine 250 may enable communication between the EMS crew and the telehealth practitioner 208.

In some implementations, after coordinating the telehealth physician support (728), the route and timing is determined to reach the patient's location (730) and transport of EMS and equipment/staffing are coordinated (732), as described above.

In some implementations, where on-site support is not deemed sufficient with or without telehealth support (726) (or telehealth support is unavailable, or the level of EMS team member required to perform the medical treatment with supervision is unavailable), it is determined whether facility transport with EMS support is sufficient (734).

In some implementations, if facility transport with EMS support is insufficient (734) telehealth physician support is coordinated (728b) for the transport. The telehealth support, for example, may be coordinated as described in relation to operation 728a.

In some implementations, whether or not telehealth physician support is coordinated (728b), route and timing are determined for the complete trip to the patient location and to a drop-off location (e.g., medical facility such as a hospital) (736). The route and timing, for example, may be determined by the navigation services engine 236 of FIG. 2.

In some implementations, transport of EMS and equipment/staffing is coordinated (732).

Although illustrated as a particular series of operations, in some embodiments, the method 700 includes more or fewer operations. For example, in addition to determining whether on-site care with telehealth (726) and/or transport with telehealth (734) best fulfills the needs of the patient, it may be determined whether resources are available to support telehealth inclusion. For example, only a portion of transport vehicles may be equipped to support telehealth supervision. Further, only a portion of crew members may have qualifications sufficient to provide interventions with the supervision of a telehealth practitioner. In these circumstances, whether or not deemed sufficient, availability of such support (e.g., by employee type, crew member certification, etc.) would need to be reviewed. In another example, the patient circumstances may not merit immediate services, either telehealth based or in person. For example, the patient may simply require instructions (monitor and call back if X gets worse or if Y doesn't improve, scheduling a visit with a primary care physician, proceed to an urgent care facility, etc.). In this situation, the caller instruction engine 240 of FIG. 2 may be selected to provide immediate instructions regarding the patient. In some embodiments, certain operations of the method 700 may be performed in a different order and/or concurrently. For example, the patient may be identified in the system (710) prior to determining the patient medical concerns (706) so that patient medical information may be used to streamline questioning (e.g., by the machine learning analysis engine 638 of FIG. 6A) to more swiftly identify a patient concern relevant to the patient based upon, in some examples, the patient's condition(s), disease(s), or recent procedure(s). Other modifications to the method 700 are possible while remaining in the spirit and scope of the method 700.

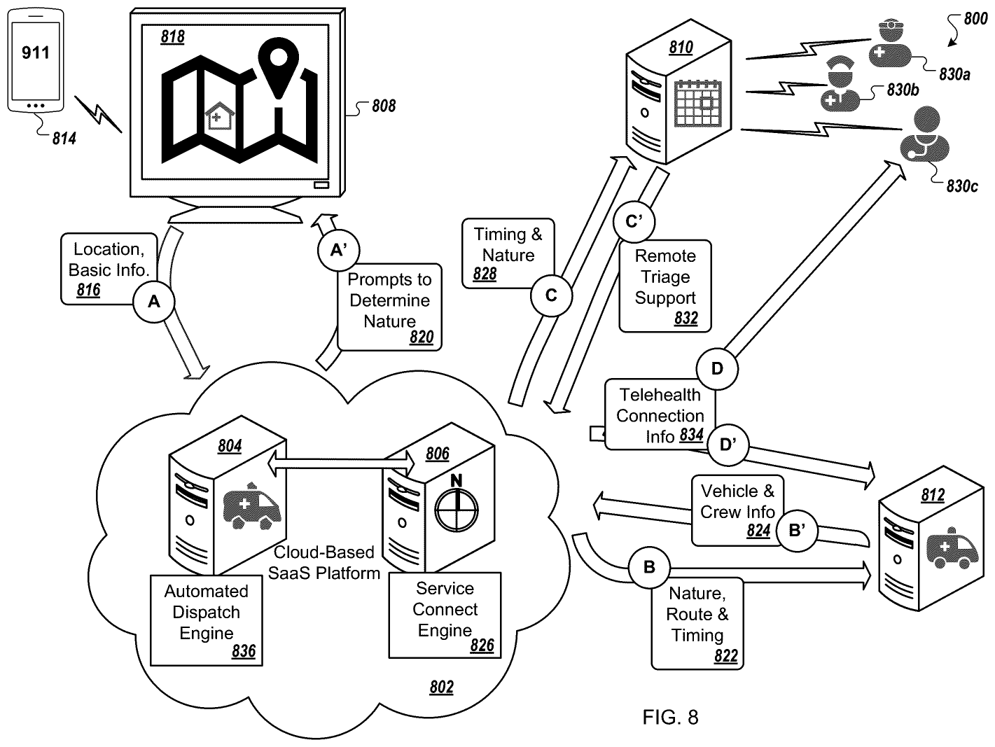

FIG. 8 is a block diagram of an example cloud-based platform 802 and environment 800 for integrating telehealth services when responding to medical emergency calls. The cloud-based software-as-a-service (SaaS) platform 802, as illustrated, includes a CAD system 804 as well as a in-vehicle dispatch system 806. However, in some embodiments, the CAD system 804 and the in-vehicle dispatch system 806 may be integrated into the same system, such as the CAD system 202 of FIG. 2. The cloud-based SaaS system 802 is in communication with a medical request intake system 808, a telehealth service scheduler 810, and a dispatch agency system 812. The medical request intake system 808 may manage intake of medical emergency calls 814. The medical request intake system 808, in some examples, may be a PSAP, part of the agency 812, part of a different agency (e.g., an originating agency which transfers the dispatch assignment to the agency 812, as described in relation to FIG. 1C), and/or part of the cloud-based SaaS platform 802. Further, although illustrated in relation medical emergency calls 814, the request intake system 808 may be a hospital-based request submission system configured to submit transfer requests to other medical facilities, such as the hospital system 108 described in relation to FIG. 1A.

In some implementations, the request intake system 808 obtains location information and basic information 816 (e.g., patient demographics, an overview of the scene) via the call 814. For example, the information may be submitted by a dispatcher at a dispatch user interface 818 (e.g., presented by the dispatch GUI engine 214 of FIG. 2, such as the call taking data entry pane 342 of the user interface display 340 of FIG. 3C).

In some implementations, the location and basic information 816 is uploaded to the cloud-based SaaS platform 802 for intake by an automated dispatch engine 836, such as the dispatch engine 216 of FIG. 2.

In some implementations, prompts 820 are provided by the cloud-based SaaS platform 802 to the request intake system 808 for determining a nature of the medical emergency. The prompts 820, for example, may be presented at the dispatch user interface 818 for a dispatcher to read to a caller. The prompts 820, for example, may be generated by the dispatch code engine 218 of FIG. 2.

In some implementations, the in-vehicle dispatch system 806 determines route and timing information related to the location and basic information 816. The route and timing information, for example, may be determined by the navigation services engine 236 of FIG. 2.

In some implementations, the nature of the medical emergency, determined via the prompts 820, along with the route and timing information, is transferred by the cloud-based SaaS platform 802 as nature, route, and timing information 822 to the agency system 812. The nature, route, and timing information 822, for example, may be used by a dispatcher to select a vehicle and crew, returning vehicle and crew information 824. The nature, route, and timing information 822, for example, may be presented in a graphical user interface such as the user interface display 350 of FIG. 3D where a dispatcher can enter an assigned resource from available resources.

In some embodiments, the automated dispatch engine 836 determines whether telehealth support is appropriate for responding to the request 814. The nature information 828, for example, may provide indications of specialties, accreditations, or other qualifications of a professional appropriate to assist in the medical emergency.

In some implementations, the automated dispatch engine 836 identifies one or more preferred telehealth professionals based on the nature information 828. The telehealth professional 830, for example, may be selected based in part on the triage practitioner data 276 of FIG. 2. For example, the triage practitioner records 276 may include a name, contact information, and one or more qualifications. Further, the triage practitioner records 276 may include availability information, for example as accessed from the telehealth service scheduler 810. The triage professional scheduling engine 248 of FIG. 2 may access a set of rules for matching medical condition descriptors (e.g., dispatch codes, symptoms, diseases and/or disorders identified from patient medical records, etc.) with qualifications of telehealth professionals as stored in the triage practitioner data 276 to determine a subset of telehealth practitioners 208 appropriate to assist in the current request.

In some implementations, if the nature is determined to be compatible with remote telehealth support of the crew, a service connect engine 826 of the in-vehicle dispatch system 806 provides timing and nature information 828 to the telehealth service scheduler 810 to obtain scheduling with one of a set of telehealth professionals 830. The service connect engine 826, for example, may identify one or more of the telehealth professionals 830 as being preferred providers for telehealth assistance.

In some implementations, the telehealth service scheduler 810 responds to the service connect engine 826 with a remote triage support appointment 832. The appointment 832 may include, in some examples, an identification of the scheduled telehealth practitioner 830, a time of availability, and/or contact information for connecting with the telehealth professional. Further, the appointment 832 may include insurance affiliations for billing purposes, a link or network address for connecting the telehealth professional 830 with the patient, and/or confirmation information for securely connecting and/or confirming the identity of the assigned telehealth professional 830.

In some implementations, the service connect engine 826 supplies telehealth connection information 834 to the selected telehealth professional 830 using the contact information provided in the appointment 832. The telehealth connection information 834, for example, may include a connection link for establishing a network connection between a computing device associated with a telehealth professional 830 and a computing device associated with an EMS crew.

In some implementations, the telehealth connection information 834 is provided to the agency 812 and/or to the assigned transport vehicle for connecting the EMS crew with the telehealth professional 830. The EMS crew, for example, may establish the connection using an on-board (e.g., installed in a transport vehicle) communication system or a portable (e.g., tablet computer, etc.) communication system temporarily associated with the transport vehicle at least for the duration of an EMS response.

The telehealth connection information 834, when applied, establishes a communication connection between the selected telehealth professional 830 and the EMS crew. The telehealth connection information 834 may establish an audio-visual link between the telehealth professional 830 and the EMS crew. Further, the telehealth connection information 834 may provide the telehealth professional 830 with data access to monitoring and/or patient record information. For example, the telehealth connection information 834 may be used to establish real-time or near real-time data transfer of monitoring data from one or more pieces of medical equipment used by the EMS crew. The monitoring data, for example, may include metrics derived from sensors of multiple medical devices. In one example, the data link may be a separate network communications link from the audio-visual link. The telehealth professional, in another example, may be provided with a videoconferencing link providing a visual feed presenting crew treatment of the patient and an audio feed of the medical scene as well as a data feed presenting monitoring feedback. In other embodiments, the service connect engine 826 may supply patient record information to the telehealth professional 830 separately.

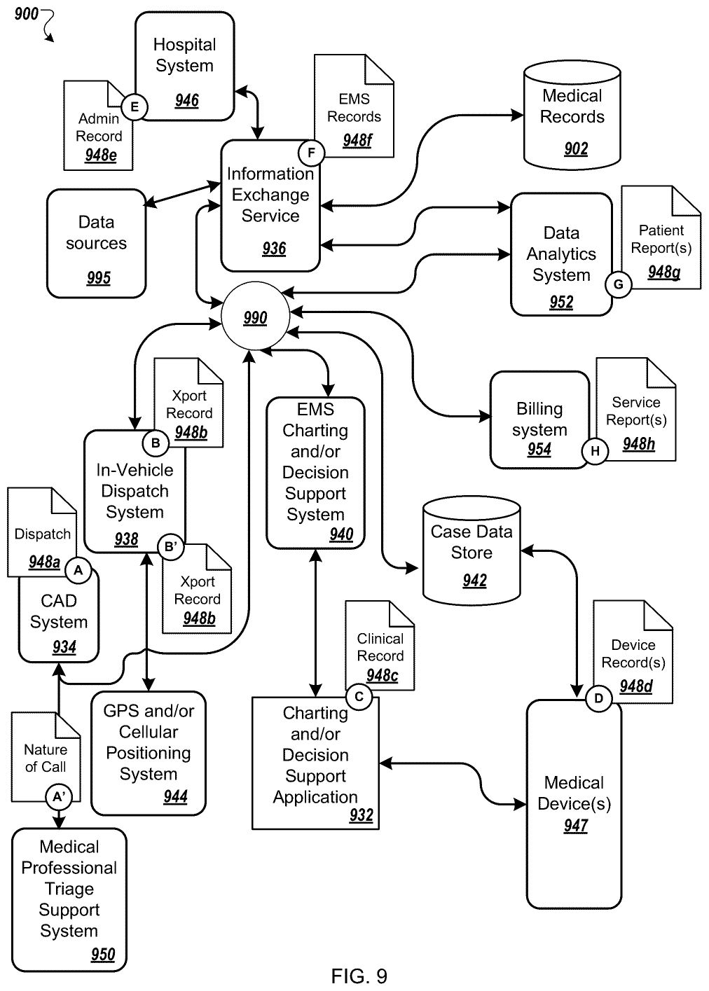

FIG. 9 is a block diagram of an example information sharing architecture 900 for sharing patient data and other information between a computer-aided dispatch system and other medical services and facilities. The architecture 900 may be implemented in a software-as-a-service cloud computing environment. Each system described below, for example, may be configured to execute on one or more servers of the cloud computing environment. It should be noted that one or more organizations may own portions of the system illustrated in FIG. 9.

In some implementations, the architecture 900 includes an information exchange service 936, a CAD system 934, an in-vehicle dispatch system 938, an EMS charting and/or decision support system 940, a hospital system 946, a data analytics system 952, a billing system 954 (e.g., medical billing system), a case data store 942, and a medical record data store 902. In some embodiments, the CAD system 934, the in-vehicle dispatch system 938, the EMS charting and/or decision support system 940, the data analytics system 952, the billing system 954 (e.g., medical billing system), the case data store 942, and the medical record data store 902 are components of a platform 990, for example a software-as-a-service (SaaS) cloud platform. Two or more of the applications associated with the SaaS cloud platform 990 may share information. The information exchange service 936 may connect the platform 990 with external records systems, such as a hospital system 946 and/or a medical record store 902. In some embodiments, the data analytics system 952 communicates with the external systems via the information exchange service 936.

In some implementations, the architecture 900 enables sharing of medical record information between entities of the architecture 900. For example, initiation of a call by the CAD system 934 (e.g., responsive to dispatch information 948a delivered to the CAD system via the information exchange service 936 or responsive to nature of call information 948i provided to the CAD system 934 via a medical professional triage support system 950) may result in initialization of a transport record 948b and/or an EMS record 948f shared via the platform 990 with the billing system 954 and/or the in-vehicle dispatch system 938. Alternatively, the CAD system 934 may communicate with the EMS charting and/or decision support system 940, and the EMS charting and/or decision support system 940 may then communicate with other elements of the architecture 900 through creating an EMS record 948f. The billing system 954 may receive the information related to the transport record 948b, dispatch record 948a, and/or EMS record 948f during or after the medical event via communications with one or more of the CAD system 934, the EMS charting and/or decision support system 940, and/or the in-vehicle dispatch system 938, as supported by the platform 990. The billing system 954, for example, may populate a service report 948h based on the information.

In some implementations, the EMS charting and/or decision support system 940 is accessed by a charting and/or decision support application 932. The charting and/or decision support application 932 may be in communication with one or more medical devices 947 utilized by an EMS crew. Each medical device 947, for example, may generate a device record 948d stored to the case data store 942. Further, the charting and/or decision support application 932 may collect information related to treatment of the patient in a clinical record 948c.

In some implementations, the patient transport vehicle used by the EMS crew includes a GPS and/or cellular positioning system 944 in communication with the in-vehicle dispatch system 938. The patient transport vehicle may be an emergency vehicle, such as an ambulance, a fire engine, a police car, and/or a helicopter.

In some implementations, the charting and/or decision support application 932 (e.g., an ePCR) is configured to execute natively on a user interface device. In other implementations, the charting and/or decision support application 932 is configured to execute within a browser and is served to a user interface device by the EMS charting and/or decision support system 940.

The charting and/or decision support application 932 may, for example, be configured to update or create an ePCR specific to a patient encounter. In some embodiments, the charting and/or decision support application 932 includes an ePCR user interface configured to interact with a user to receive patient medical information, such as charting information. The charting and/or decision support application 932, in some embodiments, is configured to upload this information, for example in the form of ePCRs, to the EMS charting and/or decision support system 940 (e.g., as clinical record 948c).

The architecture 900 may be implemented within one or more data centers or other high-capacity computing facility with high-speed internet connectivity. The architecture 900 may include a plurality of dedicated servers (e.g., a farm or cluster of computer systems) within the data center that are interconnected via a high speed, private network. Each of the systems illustrated within architecture 900 may be implemented as one or more physical and/or one or more virtual servers. The servers can include one or more application servers, web servers, and/or database servers. The servers can include enterprise servers configured to support an organization as a single tenant and/or cloud servers configured to support multiple organizations as multiple tenants.

It should be noted that the software applications hosted by servers within the architecture 900, in some embodiments, are configured to expose application programming interfaces (APIs) that enable the software applications to communicate with one another. These APIs, for example, may be configured to receive, process, and respond to commands issued by software applications hosted on the same server or a different server in the architecture 900. For example, dispatch GUIs presented to EMS agencies may be hosted dispatch GUI applications or sessions.

The APIs may be implemented using a variety of interoperability standards and architectural styles. For instance, in one example, the APIs are web services interfaces implemented using a representational state transfer (REST) architectural style. In this example, the APIs communicate with a client process using Hypertext Transfer Protocol (HTTP) along with JavaScript Object Notation (JSON) and/or extensible markup language (XML). In some examples, portions of the HTTP communications can be encrypted to increase security. Alternatively or additionally, in some examples, the APIs are implemented as a.NET web API that responds to HTTP posts to particular uniform resource locators. Alternatively or additionally, in some examples, the APIs are implemented using simple file transfer protocol (SFTP) commands and/or a proprietary application protocol accessible via a transmission control protocol socket. Thus, the APIs described herein are not limited to a particular implementation.

One or more networks enabling communication within the architecture 900 can include one or more communication networks through which the computing devices within these environments send, receive, and/or exchange data. In various implementations, the network can include a cellular communication network and/or a computer network. In some examples, the network includes and supports wireless network and/or wired connections. For instance, in these examples, the network may support one or more networking standards such as GSM, CMDA, USB, BLUETOOTH®, CAN, ZigBee®, Wireless Ethernet, Ethernet, and TCP/IP, among others. The network may include both private networks, such as local area networks, and public networks, such as the Internet. It should be noted that, in some embodiments, the network includes one or more intermediate devices involved in the routing of packets from one endpoint to another. However, in other embodiments, the network can involve only two endpoints that each have a network connection directly with the other.

In some implementations, the information exchange service 936 is configured to interoperate with the CAD system 934, the in-vehicle dispatch system 938, the EMS charting and/or decision support system 940, the billing system 954, and/or the case data store 942 to gather medical records for patients.

In some embodiments, the information exchange service 936 is configured to periodically update medical records by interoperating with the other servers in the architecture 900.

The EMS charting and/or decision support system 940 and the charting and/or decision support application 932, in some embodiments, implement an EMS patient charting application. In one example, the charting and/or decision support application 932 may receive input from EMS personnel and/or the medical devices 947 that specifies medical information regarding the patient and stores the medical information in an ePCR. The charting and/or decision support application 932 may upload ePCRs to the charting and/or decision support system 940.

The case data store 942, in some implementations, receives case files uploaded by the medical devices 947. The case data store 942 can be implemented by, for example, a database (e.g., a relational database) and stored on a non-volatile storage medium. In some embodiments, the case data store 942 includes a set of records that store case data derived from case files from medical devices used to treat patients during encounters. Moreover, in some embodiments, the case data store 942 is configured to store complete copies of the case files themselves (e.g., as large binary objects). The case data stored in the case data store 942 can document patient encounters from the point of view of the medical devices 947. As such, case data generated by a medical device during a patient encounter can include an identifier of the medical device, physiologic parameter values of the patient recorded by the medical device during the encounter, characteristics of treatment provided by the medical device to a patient during the encounter, actions taken by care providers during the encounter, and time stamps associated with medical device case data. For instance, where the medical device is a defibrillator, the case data can include patient physiological parameters such as ECG data for the patient, as well as characteristics of therapeutic shocks delivered by the defibrillator to the patient, CPR performance data, and time stamps reflecting a power-on time for the defibrillator and associated with recorded case data, among other information.

The data stores 902 and 942 can be organized according to a variety of physical and/or logical structures. In some embodiments, the data stores 902 and 942 are implemented within one or more relational databases having a highly normalized schema and accessible via a structured query language (SQL) engine, such as ORACLE or SQL-SERVER. This schema can, in some implementations, include columns and data that enable the data stores 902 and 942 to house data for multiple tenants. In addition, although the description provided above illustrates the data stores 902 and 942 as relational databases, the examples described herein are not limited to that particular physical form. Other databases may include flat files maintained by an operating system and including serialized, proprietary data structures, hierarchical database, xml files, NoSQL databases, and/or document-oriented databases. Further, to support inferences and connections within patient information, at least a portion of the data records may be stored in a neural network format. Thus, the data stores 902 and 942 are not limited to a particular implementation.

In some embodiments, the billing system 954 implements a medical billing system. The billing system 954, in some examples, can store patient identification data, information regarding claims involving patients, and/or payments status of the claims. Insurance claims may include procedure codes, for example, such as Current Procedural Terminology (CPT®) codes and/or Healthcare Common Procedure Coding System (HCPCS) codes. Rather than or in addition to the procedure codes, in some implementations, the claim information includes reference codes or identifiers not included in the CPT and/or HCPCS listings such as, in some examples, medical equipment and/or supply identifiers, prosthetics and/or orthotics devices and supplies identifiers, home health services identifiers, and/or prescription drug identifiers. The patient identification data stored in the billing system 954 can include, for example, patient provider and insurance information.

In some embodiments, the hospital system 946 originates administrative records 948e for sharing with the information exchange service 936. The hospital system 946, for example, may obtain medical information regarding a patient from the medical records store 902 via the information exchange service 936 and add the patient information (e.g., demographics information, etc.) into a new administrative record 948e.

In some embodiments, a data analytics system 952 may derive insights from data accessed from a variety of disparate data sources 995 accessed via the information exchange service 936 such as, in some examples, insurance companies, credit records, and/or medical records. These data sources may provide, for example, patient financial data, medical payer data, credit score data, patient demographic data, historic medical claims data, medical provider data, etc. The data analytics system 952 may apply machine learning analysis and/or or other statistical data analysis techniques to identify patterns in the various data records to produce complex predictive intelligence, for example by deriving insights, metrics and/or calculating estimates using the data from the disparate data sources. The output of the data analytics system 952 may include predictive intelligence regarding remittance value for medical services from individual payers or for combined payers (e.g., primary insurance plus secondary insurance, primary insurance plus patient deductible value, etc.). This information may include predicted payment schedules, prior authorization information or deductible information. In some implementations, the data analytics system 952 provides demographic verification and insurance discovery tools. The data analytics system 952 may provide predictive intelligence output 948g in various formats, including stored data files or data rendered in a graphical user interface in the form of short textual and/or numerical answers, charts, graphs, interactive spreadsheets, and/or interactive reports, in some examples.

In some implementations, the medical device(s) 947 include a patient treatment device, or another kind of device that includes patient monitoring and/or patient treatment capabilities. For example, the medical device(s) 947 may include a defibrillator configured to deliver therapeutic electric shocks to the patient. In other examples, the medical device(s) 947 can deliver other types of treatments, such as ventilation, operating a respirator, and/or administering drugs or other medication.

In combination, the architecture 900 can produce accurate and comprehensive documentation that improves continuity of patient care and overall patient health outcomes. More specifically, continuity of care may benefit from a record that thoroughly describes symptoms, physiological scores, and treatments provided.

Reference has been made to illustrations representing methods and systems according to implementations of this disclosure. Aspects thereof may be implemented by computer program instructions. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, the process 400 of FIG. 4A, the method 500 of FIG. 5A and FIG. 5B, the process 600 of FIG. 6A, the process 660 of FIG. 6B, and/or the method 700 of FIG. 7A and FIG. 7B.

These computer program instructions can direct a computing device or other programmable data processing apparatus to function in a particular manner, such that the instructions stored in the computer-readable medium produce an article of manufacture including instruction means which implement the function/operation specified in the illustrated process flows.

The engines described herein (e.g., the various engines shown in FIG. 1C, FIG. 2, FIG. 4B, FIG. 6A, FIG. 6B, and FIG. 8) may include hardware logic and/or software logic provided by a processor and/or the memory to implement logic instructions to perform one or more tasks at a computing device and/or at a medical device. In some examples, the actions performed by engines may be implemented by hardware, software, firmware, middleware, microcode, hardware description languages, etc. or any combination thereof. When implemented in software, firmware, middleware, or microcode, the program code or code segments to perform the tasks may be stored in a non-transitory processor-readable medium such as a storage medium. Processors may perform the described tasks. In various implementations, the engines described herein may be or may include at least one processor and a non-transitory processor-readable storage medium having stored thereon processor-readable instructions configured to cause the at least one processor to perform actions according to the processor-readable instructions. The storage medium and/or the processor may be components of a computing device and/or a medical device.

Embodiments of the present description rely on network communications. As can be appreciated, the network can be a public network, such as the Internet, or a private network such as a local area network (LAN) or wide area network (WAN) network, or any combination thereof and can also include PSTN or ISDN sub-networks. The network can also be wired, such as an Ethernet network, and/or can be wireless such as a cellular network including EDGE, 3G, 4G, and 5G wireless cellular systems. The wireless network can also include Wi-Fi®, Bluetooth®, Zigbee®, or another wireless form of communication. The network, for example, may support communications between the CAD system 104 and the in-vehicle dispatch system 106, the hospital 108 and the cloud-based SaaS platform 102, the agency 150 and the CAD system 104, and/or the transport vehicle 124 and the in-vehicle dispatch system 106 of FIG. 1A, the PSAP 146 and the agencies 150 and/or the CAD system 104a of FIG. 1B, the CAD system 104a and the agencies 150a of FIG. 1B, the agencies 150 and the CAD system 104b of FIG. 1C, the CAD system 202 and the agencies 204, facilities 206, telehealth practitioners 208, and/or transport vehicles 210 of FIG. 2, the agency computer 458 and the cloud-based CAD system 480 of FIG. 4B, and/or the cloud-based SaaS platform 802 and the medical request intake system 808, the telehealth service scheduler 810, and/or the agency 812 of FIG. 8.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display. The display controller and display may enable presentation of the screenshots illustrated, in some examples, in FIG. 3A through FIG. 3E.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

The functions and features described herein may also be executed by various distributed components of a system. For example, one or more processors may execute these system functions, where the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system, in some examples, may be received via direct user input and/or received remotely either in real-time or as a batch process.

Although provided for context, in other implementations, methods and logic flows described herein may be performed on modules or hardware not identical to those described. Accordingly, other implementations are within the scope that may be claimed.

In some implementations, a cloud computing environment, such as Google Cloud Platform™ or Amazon™ Web Services (AWS™), may be used perform at least portions of methods or algorithms detailed above. The processes associated with the methods described herein can be executed on a computation processor of a data center. The data center, for example, can also include an application processor that can be used as the interface with the systems described herein to receive data and output corresponding information. The cloud computing environment may also include one or more databases or other data storage, such as cloud storage and a query database. In some implementations, the cloud storage database, such as the Google™ Cloud Storage or Amazon™ Elastic File System (EFS™), may store processed and unprocessed data supplied by systems described herein. For example, the contents of the data store 170 of FIG. 1C, the data repository 212 of FIG. 2, the data repository 622 of FIG. 6A, the data stores 622, 664, 666, and/or 670 of FIG. 6B, and/or the data stores 902 and/or 942 of FIG. 9 may be maintained in a database structure.

The systems described herein may communicate with the cloud computing environment through a secure gateway. In some implementations, the secure gateway includes a database querying interface, such as the Google BigQuery™ platform or Amazon RDS™. The data querying interface, for example, may support access by the CAD system 104b of FIG. 1C, the CAD system 202 of FIG. 2, the caller interrogation engine 604 and/or the machine learning analysis engine 638 of FIG. 6A, the ML model training engine 662 and/or the ML model updating engine 672 of FIG. 6B, and/or the systems of the architecture 900 of FIG. 9.

In some embodiments, an edge server is used to transfer data between the CAD system 202 and the computing systems associated with agencies 204, facilities 206, telehealth practitioners 208, and/or transport vehicles 210 and/or the various systems of the data sharing architecture 9 of FIG. 9 according to various embodiments described herein. The edge server, for example, may be a computing device configured to execute processor intensive operations that are sometimes involved when executing machine learning processes, such as natural language processing (NLP) operations. An edge server may include, for example, one or more GPUs that are capable of efficiently executing matrix operations as well as substantial cache or other high-speed memory to service the GPUs. An edge server may be a separate, ruggedized physical device that travels with EMS personnel in the field. An edge server may be incorporated into other EMS field equipment such as a medical device or patient monitoring device. Alternatively or additionally, an edge server may be located within a carrying case for a medical device. An edge server, in a further example, may be incorporated into the communications and processing capabilities of an EMS vehicle or otherwise be located within the EMS vehicle.

In some implementations, certain devices described herein operate as the edge server, for example if the processing capability of these devices is sufficient to provide computing services associated with an edge server. The edge server, in some embodiments, can be used to move more computing capability into the local environment so that any computation intensive data processing and/or analytics can run accurately and efficiently. An edge server may be incorporated into a device or added as another device to support local devices in the absence of a connection with a remote cloud server.

Regardless of its physical form, an edge server can be configured to interoperate with other devices proximate or local to the edge server, directly or via a network. For instance, the edge server can include a wireless network interface (e.g., a PAN interface, LAN interface, WAN interface, or the like) through which the edge server can communicate with the other devices at the scene and/or a server (e.g., cloud) environment.

In a reciprocal manner, the devices at an EMS scene, in some embodiments, are configured to connect directly or indirectly to and interoperate with the edge server via a short-range wireless connection, such as a PAN connection or a LAN connection. In an illustrative example, the medical devices 947 and/or the charting and/or decision support system 940 of FIG. 9 may communicate via a short-range wireless connection to an edge server and, in turn, the edge server may communicate via a long-range wireless connection to the information exchange service 936.

In some implementations, the method 500 of FIG. 5A and FIG. 5B, the process 600 of FIG. 6A, the process 660 of FIG. 6B, and/or the method 700 of FIG. 7A and FIG. 7B may be configured to be performed in part by an edge server or a device interoperating with an edge server. The device interoperating with the edge server, for example, may share processing functionality with the edge server via one or more APIs implemented by the processes.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1.-159. (canceled)

160. A cloud-based computer-aided dispatch (CAD) system comprising at least one non-transitory, processor-readable storage medium having stored thereon processor-readable instructions for emergency medical services (EMS) dispatch services, the processor-readable instructions being configured to cause at least one processor to:
communicatively couple with a mobile computing device associated with an EMS agency vehicle;
receive real-time EMS vehicle location tracking information from the mobile computing device associated with the EMS agency vehicle;
receive a request for EMS services for a patient;
generate a pending EMS trip assignment in response to the request for EMS services;
communicatively couple to a computing system associated with an EMS agency via a long-range communications network;
host a dispatch graphical user interface (GUI) at the computing system associated with the EMS agency, wherein the dispatch GUI comprises:
an interactive graphical map, and
a data entry window comprising a plurality of data entry fields;
automatically capture at least one item of EMS trip information in at least one data entry field of the plurality of data entry fields based on a user selection of a position or icon from the interactive graphical map;
populate the pending EMS trip assignment based at least on part on the automatically captured at least one item of EMS trip information, the real-time EMS vehicle location tracking information, and the request for the EMS services for the patient, wherein the pending EMS trip assignment comprises an identification of at least one EMS agency vehicle;
send the pending EMS trip assignment to the mobile computing device associated with the EMS agency vehicle;
receive a trip acceptance from the mobile computing device associated with the EMS agency vehicle; and
generate an EMS agency vehicle assignment of the EMS agency vehicle to the request for EMS services in response to the trip acceptance;
wherein the cloud-based CAD system is configured for execution on a cloud-based distributed processing architecture.

161. The cloud-based CAD system of claim 160, wherein the processor-readable instructions are configured to cause the at least one processor to:
identify a location corresponding to the position on the interactive graphical map; and
display the identified location in the at least one data entry field configured to automatically capture the item of EMS trip information based on the user selected position on the interactive graphical map.

162. The cloud-based CAD system of claim 160, wherein:
the at least one data entry field is a patient transport location comprising a patient drop-off location for the EMS trip or a patient pick-up location for the EMS trip; and
the processor-readable instructions are configured to cause the at least one processor to provide a transport location icon on the interactive graphical map at a map location corresponding to a geographic location of the patient transport location, and
automatically populate the at least one data entry field with the patient transport location based on a user selection of the transport location icon.

163. The cloud-based CAD system of claim 162, wherein the processor-readable instructions are configured to cause the at least one processor to:
accept a repositioning of the transport location icon at the interactive graphical map; and
update the at least one data entry field to correspond with the repositioning.

164. The cloud-based CAD system of claim 160, wherein the user selection of the position on the interactive graphical map causes the at least one processor to automatically capture the item of EMS trip information as global positioning satellite (GPS) coordinates for the position.

165. The cloud-based CAD system of claim 160, wherein the processor-readable instructions are configured to cause the at least one processor to:
provide a plurality of icons representing agency resource locations on the interactive graphical map; and
display agency resource information at the dispatch GUI for a particular location in response to a user selection of a respective icon of the plurality of icons, wherein the respective icon is provided at the particular location.

166. The cloud-based CAD system of claim 165, wherein the processor-readable instructions are configured to cause the at least one processor to:
access at least one post location for the EMS agency; and
provide at least one post location icon on the interactive graphical map at the at least one post location.

167. The cloud-based CAD system of claim 166, wherein the at least one post location icon indicates a quantity of vehicles currently located at the at least one post location.

168. The cloud-based CAD system of claim 166, wherein the processor-readable instructions are configured to cause the at least one processor to automatically capture the item of EMS trip information based on a user selection of at least one post location icon.

169. The cloud-based CAD system of claim 165, wherein the processor-readable instructions are configured to cause the at least one processor to:
provide a set of individual EMS vehicle icons at the interactive graphical map based on the real-time EMS vehicle location tracking information, each EMS vehicle icon representing a respective EMS vehicle for the EMS agency; and
automatically capture the item of EMS trip information based on a user selection of at least EMS vehicle icon.

170. The cloud-based CAD system of claim 165, wherein:
the plurality of icons comprises at least one post location icon and at least one individual EMS vehicle location icon; and
the processor-readable instructions are configured to cause the at least one processor to:
provide the at least one post location icon with a first shape and a first color; and
provide the at least one individual EMS vehicle location icon with a second shape and a second color, wherein
the second shape is different from the first shape or the second color is different from the first color or a combination thereof.

171. The cloud-based CAD system of claim 165, wherein the processor-readable instructions are configured to cause the at least one processor to display one or more of crew information or vehicle information in response to a user selection of a particular icon of the plurality of icons.

172. The cloud-based CAD system of claim 171, wherein the vehicle information comprises a connectivity status representing one of availability of communications with the mobile computing device associated with the EMS agency vehicle or unavailability of communications with the mobile computing device associated with the EMS agency vehicle, wherein the EMS agency vehicle corresponds to the particular icon.

173. The cloud-based CAD system of claim 165, wherein the processor-readable instructions are configured to cause the at least one processor to provide filter options for the interactive graphical map that enable a user to filter display of the plurality of icons to a particular type of EMS vehicle.

174. The cloud-based CAD system of claim 160, wherein the processor-readable instructions are configured to cause the at least one processor to display a log of the EMS trip, the log comprising
a current status of an EMS agency vehicle corresponding to the EMS agency vehicle assignment,
a time of the generation of the EMS agency vehicle assignment, and
a time interval from the generation of the EMS agency vehicle assignment to a wheels rolling status.

175. The cloud-based CAD system of claim 174, wherein the log comprises one or more of a time or time interval from the generation of the EMS agency vehicle assignment for an en route status, an at-scene status, a transporting status, or an at-destination status.

176. The cloud-based CAD system of claim 174, wherein the log of the EMS trip comprises a time for a pre-scheduled patient transport.

177. The cloud-based CAD system of claim 174, wherein the log of the EMS trip comprises one or more of a vehicle identification number, a vehicle service level, crew member identification, contact information for a requester of the EMS services, or a connectivity status representing communications availability or unavailability between the mobile computing device and the cloud-based CAD system.

178. The cloud-based CAD system of claim 177, wherein:
the at least one processor is configured to interoperate with an in-vehicle dispatch system provided in the cloud-based distributed processing architecture and configured to provide a navigation and dispatch application at the mobile computing device associated with the EMS agency vehicle; and
the connectivity status registers unavailability between the mobile computing device and the cloud-based CAD system prior to a crew member of the EMS agency vehicle logging into the navigation and dispatch application.

179. The cloud-based CAD system of claim 174, wherein the log of the EMS trip comprises a time stamp control, a cancel trip control, an unassign trip control, an add trip control, a return trip control, and an add leg control.

180. The cloud-based CAD system of claim 179, wherein the time stamp control, upon selection, is configured to enable a user to enter a custom time stamp comprising a clock time and a text label, wherein the text label represents an action, status, or activity corresponding to the clock time.

181. The cloud-based CAD system of claim 174, wherein the processor-readable instructions are configured to cause the at least one processor to display the log of the EMS trip in a scrollable window.

182. The cloud-based CAD system of claim 160, wherein:
the EMS agency is a first EMS agency at a first geographic location;
the dispatch GUI is a first dispatch GUI;
the system comprises a database of EMS vehicles associated at least with the first EMS agency and a second EMS agency at a second geographic location; and
the processor-readable instructions are configured to cause the at least one processor to identify an EMS vehicle associated with the second EMS agency that is available for the EMS trip based on at least one dispatch code,
receive a patient location associated with the EMS trip,
determine that the second geographic location is closer to the patient location than the first geographic location,
transfer the pending EMS trip assignment from the first EMS agency to the second EMS agency, and
provide a transfer notification for the EMS trip at a second dispatch GUI at a computing system located at the second EMS agency.

183. The cloud-based CAD system of claim 182, wherein the transfer notification comprises an assignment of an EMS agency vehicle associated with the second EMS agency to the EMS trip.

184. The cloud-based CAD system of claim 182, wherein the transfer notification comprises a request for the second EMS agency to allocate resources to the pending EMS trip assignment without an assignment of the EMS agency vehicle associated with the second EMS agency.

185. The cloud-based CAD system of claim 182, wherein the database of EMS vehicles comprises stored permission information that enables the at least one processor to transfer the EMS trip from the first EMS agency to the second EMS agency.

186. The cloud-based CAD system of claim 185, wherein the stored permission information comprises data access guidelines that identify one or more types of EMS trip data that are exchangeable between the first EMS agency and the second EMS agency.

187. The cloud-based CAD system of claim 182, wherein a computing system located at the second EMS agency and the computing system located at the first EMS agency are separate uncoupled computing systems such that a data exchange between the first EMS agency and the second EMS agency only occurs via the cloud-based distributed processing architecture of the cloud-based CAD system.

188. The cloud-based CAD system of claim 182, wherein the processor-readable instructions are configured to cause the at least one processor to convert EMS trip data from a first format or dispatch workflow associated with the first EMS agency to a second format or dispatch workflow associated with the second EMS agency.

189. The cloud-based CAD system of claim 160, wherein:
the at least one processor is configured to interoperate with an in-vehicle dispatch system provided in the cloud-based distributed processing architecture and configured to provide a navigation and dispatch application at the mobile computing device associated with the EMS agency vehicle; and
the dispatch GUI is configured to receive the trip acceptance from a user entry to the navigation and dispatch application and provide the EMS agency vehicle assignment to the navigation and dispatch application.
